(19) United States
(12) Patent Application Publication
(10) Pub. No.: US 2026/0295460 A1
(43) Pub. Date: Oct. 1, 2026

(54) PROCESS FOR PREPARING EXTRACELLULAR VESICLES

(71) Applicant: LONZA SALES AG, Basel (CH)

(72) Inventors: Andrew WOOD, Basel (CH); Michael F. DOHERTY, Basel (CH); Matt BROWN, Basel (CH); Aaron NOYES, Basel (CH); Yaozhong ZHANG, Basel (CH)

(21) Appl. No.: 19/479,439

(22) PCT Filed: Apr. 23, 2024
(86) PCT No.: PCT/US2024/025793

§ 371 Date: Oct. 28, 2025

Related U.S. Application Data
(60) Provisional application No. 63/499,032, filed on Apr. 28, 2023.

(51) Int. Cl. B01D 15/20; B01D 15/36; B01D 15/38; B01D 15/42

(52) U.S. Cl. CPC B01D 15/20; B01D 15/362; B01D 15/363; B01D 15/3804; B01D 15/426

(57) ABSTRACT

The present disclosure relates to multistep chromatographic methods for preparing extracellular vesicles (EVs). The methods were demonstrated to be effective in preparing high-quality EVs in a large scale. The methods enable preparation of EVs for therapeutic and diagnostic applications, and isolation and/or sub-fractionation of EVs with desired properties for specific use.

### FIELD OF DISCLOSURE

The present disclosure provides multistep enzymatic and chromatographic methods for preparing extracellular vesicles (EVs). The methods are effective in preparing high-quality EVs, with low levels of contaminating nucleic acid molecules, on a large scale.

### BACKGROUND OF DISCLOSURE

Extracellular vesicles (EVs) are important mediators of intercellular communication. They are also important biomarkers in the diagnosis of many diseases, such as cancer. As drug delivery vehicles, EVs offer many advantages over traditional drug delivery methods, especially for gene therapy. The use of EVs for therapeutic purposes requires that EVs be free or mostly free of impurities including, but not limited to, undesirable nucleic acid molecules (e.g., DNA), host cell proteins, carbohydrates, and lipids. Current purification methods do not offer sufficient selectivity to remove significant amounts of these impurities so additional processes are desired to improve purity.

Furthermore, synthetic nano- and/or micro-carriers such as EVs often struggle to meet clinical expectations because of heterogeneity in their physicochemical parameters that confer targeting efficiency, immune evasion, and controlled drug release. This is mainly due to the complexity of nanoparticle properties (composition, size, shape, rigidity, surface charge, hydrophilicity, stability, and ligand type and density), payload properties (drug type, solubility, loading, potency, dosing, immune response, and release kinetics), and in vivo physiological barriers to nanoparticle trafficking (immune surveillance, particle extravasation, tissue targeting, tissue penetration, and cellular uptake). Although a considerable amount of effort has been made, effective methods for isolating discrete sub-populations of EVs (especially at scale) are not yet readily available.

In addition, therapeutic use of EVs requires larger-scale production and preparation of EVs. The heterogeneity and complexity of EVs make it difficult and costly to provide EVs in a large amount, while ensuring their quality. Inherent variability of the production and preparation process make it both expensive and unpredictable.

Therefore, effective and efficient methods for large-scale production, isolation and/or sub-fractionation of EVs are needed to enable use of EVs for therapeutic purposes.

### SUMMARY OF DISCLOSURE

Some aspects of the present disclosure are directed to a method of preparing purified extracellular vesicles (EVs) from a sample comprising EVs and one or more nucleic acid molecules, comprising contacting a chromatography resin associated with the sample with a wash buffer; wherein the wash buffer comprises MgCl₂, CaCl₂, KCl, and/or NaCl and does not comprise a nuclease.

Some aspects of the present disclosure are directed to a method of reducing the concentration of residual nucleic acid molecule in a sample comprising extracellular vesicles (EVs), comprising contacting a chromatography resin associated with the sample with a wash buffer; wherein the wash buffer comprises MgCl₂, CaCl₂, KCl, and/or NaCl and does not comprise a nuclease.

In some aspects, the sample does not come into a contact with a nuclease during the method.

In some aspects, the chromatography resin is selected from the group consisting of a cation exchange resin, an anion exchange (AEX) resin, an affinity chromatography resin, a pseudo affinity chromatography resin, a hydrophobic interaction resin, a hydrophobic charge induction chromatography resin, a mixed mode resin, an immobilized metal affinity resin, a ceramic hydroxyapatite resin, a fluoro hydroxyapatite resin, a ceramic fluoroapatite, and any combination thereof. In some aspects, the chromatography resin comprises an AEX resin. In some aspects, the chromatography resin comprises a CEX resin. In some aspects, the chromatography resin comprises an affinity chromatography resin.

In some aspects, the nuclease is an endonuclease or exonuclease. In some aspects, the nuclease is selected from salt active nuclease (SAN), benzonase, denarase, kryptonase, and any combination thereof.

In some aspects, the wash buffer comprises MgCl₂, and wherein the concentration of Mg²⁺ in the wash buffer is at least about 200 mM to about 500 mM. In some aspects, the wash buffer comprises MgCl₂, and wherein the concentration of Mg²⁺ in the wash buffer is at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, or at least about 500 mM Mg²⁺. In some aspects, the concentration of the Mg²⁺ in the wash buffer is at least about 350 mM Mg²⁺. In some aspects, the wash buffer comprises at least about 350 mM MgCl₂.

In some aspects, the wash buffer comprises CaCl₂, and wherein the concentration of Ca²⁺ in the wash buffer is at least about 100 mM to about 400 mM. In some aspects, the wash buffer comprises CaCl₂, and wherein the concentration of Ca²⁺ in the wash buffer is at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, or at least about 400 mM Ca²⁺. In some aspects, the concentration of the Ca²⁺ in the wash buffer is at least about 350 mM Ca²⁺. In some aspects, the wash buffer comprises at least about 350 mM CaCl₂. In some aspects, the concentration of the Ca²⁺ in the wash buffer is at least about 250 mM Ca²⁺. In some aspects, the wash buffer comprises at least about 250 mM CaCl₂.

In some aspects, the wash buffer comprises at least about 600 mM to about 1 M NaCl. In some aspects, the wash buffer comprises NaCl at a concentration of at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1 M NaCl. In some aspects, the wash buffer comprises at least about 800 mM NaCl.

In some aspects, the wash buffer comprises at least about 600 mM to about 1 M KCl. In some aspects, the wash buffer comprises KCl at a concentration of at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1 M KCl. In some aspects, the wash buffer comprises at least about 800 mM KCl.

In some aspects, the wash buffer is contacted with the chromatography resin associated with the sample at least 2 times, at least 3 times, at least 4 times, or at least 5 times.

In some aspects, the method further comprises contacting the chromatography resin associated with the sample with a second wash buffer, wherein the second wash buffer does not comprise a nuclease. In some aspects, the second wash buffer is different from the wash buffer.

In some aspects, the second wash buffer comprises MgCl₂, CaCl₂, KCl, and/or NaCl. The method of any one of claims 22 to 24, wherein the second wash buffer comprises at least about 600 mM to about 1 M NaCl. In some aspects, the second wash buffer comprises NaCl at a concentration of at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1 M NaCl. In some aspects, the second wash buffer comprises at least about 800 mM NaCl.

In some aspects, the wash buffer comprises MgCl₂, wherein the concentration of Mg²⁺ in the wash buffer is at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, or at least about 500 mM Mg²⁺; and the second wash buffer comprises at least about 600 mM to about 1 M NaCl.

In some aspects, the wash buffer comprises MgCl₂, wherein the concentration of Mg²⁺ in the wash buffer is at least about 350 mM Mg²⁺, and the concentration of NaCl in the second wash buffer is about 800 mM NaCl.

In some aspects, the wash buffer comprises at least about 600 mM to about 1 M NaCl; and the second wash buffer comprises MgCl₂, wherein the concentration of Mg²⁺ in the wash buffer is at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, or at least about 500 mM Mg²⁺. In some aspects, the wash buffer comprises at least about 800 mM NaCl; and the second wash buffer comprises MgCl₂, wherein the concentration of Mg²⁺ in the wash buffer is at least about 350 mM.

In some aspects, the second wash buffer comprises at least about 600 mM to about 1 M KCl. In some aspects, the second wash buffer comprises at least about 600 mM to about 1 M KCl. In some aspects, the second wash buffer comprises KCl at a concentration of at least about 600 mM, at least about 625 mM, at least about 650 mM, at least about 675 mM, at least about 700 mM, at least about 725 mM, at least about 750 mM, at least about 775 mM, at least about at least about 800 mM, at least about 825 mM, at least about 850 mM, at least about 875 mM, at least about 900 mM, at least about 925 mM, at least about 950 mM, at least about 975 mM, or at least about 1 M KCl. In some aspects, the second wash buffer comprises at least about 800 mM KCl.

In some aspects, the method further comprises eluting the EVs from the chromatography resin by contacting the chromatography resin associated with a sample with an elution buffer, wherein the eluting occurs after contacting the chromatography resin with the wash buffer. In some aspects, the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M. In some aspects, the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M NaCl. In some aspects, the elution buffer comprises at least about 1.2 M NaCl. In some aspects, the elution buffer comprises at least about 1.4 M NaCl.

In some aspects, the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M KCl. In some aspects, the elution buffer comprises at least about 1.2 M KCl. In some aspects, the elution buffer comprises at least about 1.4 M KCl.

In some aspects, the elution buffer releases one or more EVs from the chromatography resin. In some aspects, the method further comprises collecting an eluent after contacting the chromatography resin with the elution buffer. In some aspects, the eluent comprises one or more EVs.

In some aspects, the sample associated with the chromatography resin comprises a starting concentration of the one or more nucleic acid molecules, and wherein the eluent comprises an eluted concentration of the one or more nucleic acid molecules, wherein the eluted concentration of the one or more nucleic acid molecules is less than about 10%, less than about 5%, less than about 1%, less than about 0.5%, less than about 0.1%, less than about 0.05%, less than about 0.01%, less than about 0.001%, or less than about 0.0001% that of the starting concentration of the one or more nucleic acid molecules.

In some aspects, the method further comprises subjecting the sample to one or more additional chromatography resins. In some aspects, the one or more additional chromatography resins comprises an anion exchange chromatography (AEX) resin, a cation exchange chromatography (CEX) resin, a mixed mode chromatography (MMC) resin, hydrophobic charge induction chromatography resin, a hydrophobic interaction chromatography resin, an immobilized metal affinity chromatography (IMAC), a ceramic hydroxyapatite resin, a fluoro hydroxyapatite resin, a ceramic fluoroapatite resin, an affinity chromatography resin, a pseudo affinity chromatography resin, or any combination thereof.

In some aspects, the EV is an exosome.

Some aspects of the present disclosure are directed to a composition comprising extracellular vesicles prepared by a method disclosed herein. In some aspects, the composition further comprises a saccharide, sodium chloride, a potassium phosphate, a sodium phosphate, and any combination thereof.

Some aspects of the present disclosure are directed to a method of treating a disease or condition in a subject in need thereof comprising administering a composition disclosed herein.

### BRIEF DESCRIPTION OF THE FIGURES

FIGS. 1A-1B are graphical representations of the concentration of material eluted from an AEX matrix loaded with a sample comprising exosomes, following a first wash comprising 350 mM MgCl₂ (Wash 1), a second wash comprising 600-1100 mM NaCl (Wash 2) (FIG. 1A), and final elution (FIGS. 1A-1B).

FIGS. 2A-2C are graphical representations of DNA concentration (ng/mL) and NTA yield following elution from an AEX matrix loaded with a sample comprising exosomes, where the AEX matrix was washed with 350 mM MgCl₂ (Wash 1) followed by a second wash comprising increasing concentrations of CaCl₂ (FIG. 2A), L-arginine (FIG. 2B), or NaCl (FIG. 2C). FIG. 2D is a graphical representation of the concentration of PTGFRN in samples following elution from an AEX matrix loaded with a sample comprising exosomes, where the AEX matrix was washed with 350 mM MgCl₂ (Wash 1) followed by a second wash comprising increasing concentrations of CaCl₂ or L-arginine, as indicated. Controls are shown in the table below the graph.

FIG. 3 is a graphical representation of eluate particle size following elution from an AEX matrix loaded with a sample comprising exosomes, where the AEX matrix was washed with 350 mM MgCl₂ (Wash 1) followed by a second wash comprising increasing concentrations of CaCl₂ (circles), L-arginine (X's), or NaCl (diamonds). Controls are shown in the table below the graph.

FIGS. 4A-4D are graphical representations of elution Protein X concentration (FIG. 4A), agrin concentration (FIG. 4B), yield (FIG. 4C), and DNA concentration (FIG. 4D) in the eluate following elution from an AEX matrix loaded with a sample comprising exosomes, where the AEX matrix was washed with 350 mM MgCl₂ (Wash 1) followed by a second wash comprising increasing concentrations of NaCl (Wash 2).

FIGS. 5A-5B are graphical representations of residual DNA of exosome intermediates (FIG. 5A) and log reduction value (LRV) DNA per unit operation (FIG. 5B) following various stages exosome purification.

### DETAILED DESCRIPTION OF DISCLOSURE

The present disclosure provides methods of preparing purified extracellular vesicles (EVs) from a sample comprising EVs and one or more nucleic acid molecules. The EVs processed by the present methods can be highly purified, e.g., less nucleic acid molecule impurities, higher potency, higher uniformity, or any combination thereof.

Certain aspects of the present disclosure are directed to methods of preparing purified EVs from a sample comprising EVs and one or more nucleic acid molecules, comprising contacting a chromatography resin associated with the sample with a wash buffer; wherein the wash buffer comprises MgCl₂, CaCl₂, KCl, and/or NaCl and does not comprise a nuclease. Use of an endonuclease is the gold standard for reducing or removing nucleic acid molecules from a sample. However, nuclease treatment and its subsequent removal add time to the manufacturing process, and it can be difficult to ensure that no residual nuclease remains. The present disclosure provides novel methods of reducing or eliminating contaminating nucleic acid molecules from a sample without the use of a nuclease. Some aspects of the present disclosure are directed to methods of reducing the concentration of residual nucleic acid molecule in a sample comprising EVs, comprising contacting a chromatography resin associated with the sample with awash buffer; wherein the wash buffer comprises MgCl₂, CaCl₂, KCl, and/or NaCl and does not comprise a nuclease. In some aspects, the sample does not come into a contact with a nuclease during the method.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values, which are about the same quantity or amount as the recited value are also within the scope of the disclosure. Where a combination is disclosed, each sub-combination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, and U represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “large scale” refers to a production scale that is larger than an experimental or laboratory use for research purposes only. Large scale purification is the final production step, prior to product formulation, in the manufacture of therapeutic products, e.g., EVs. Large-scale purification requires a scale-up from laboratory scale techniques to satisfy the need for larger amounts of extremely pure test quantities of the product for analysis, characterization, testing of efficacy, clinical or field trials, and, finally, full scale commercialization. The uncompromising standards for product quality, as well as rigorous quality control of manufacturing practices embodied in current good manufacturing practices (cGMP's), provide further challenges to the scale-up of EV purification. Analysis of electrokinetic, chromatographic, adsorptive, and membrane separation techniques suggests that if yield recovery is paramount, documented purity is critical, and both must ultimately be attained within certain cost constraints. The term “large scale” as used herein indicates that the final product is for use in clinical settings and commercial sales of the purified EV products. In some aspects, the term “large scale” purification means a purification process of at least about 500 L, at least about 550 L, at least about 600 L, at least about 650 L, at least about 700 L, at least about 750 L, at least about 800 L, at least about 850 L, at least about 900 L, at least about 950 L, at least about 1000 L, at least about 1500 L, or at least about 2000 L cell culture harvest. In some aspects, the term “large scale” purification means a purification process of at least about 2000 L cell culture harvest. In some aspects, the term “large scale” purification means a purification process of at least about 3000 L, at least about 4000 L, at least about 5000 L, at least about 6000 L, at least about 7000 L, at least about 8000 L, at least about 9000 L, at least about 10,000 L, at least about 11,000 L, at least about 12000 L, at least about 13,000 L, at least about 14,000 L, or at least about 15,000 L cell culture harvest.

As used herein, the terms chromatography “resin” and “matrix” are used interchangeably, and refer to the stationary (e.g., solid) phase of a chromatography (e.g., a column chromatography or a membrane chromatography). The methods disclosed herein can be applied to any form of chromatography suitable for the purification of EVs, e.g., exosomes. In some aspects, the chromatography resin is present in a packed column (e.g., a packed-bed column chromatography). In some aspects, the chromatography resin is present on a convective media (e.g., a membrane (e.g., a membrane chromatography), one or more monoliths, one or more fibers, or any combination thereof). In certain aspects, the chromatography resin comprises an “affinity” chromatography resin, which refers to a chromatography resin that interacts with one or more molecules present in the mobile phase of the chromatography. An affinity chromatography can be used in a “bind-and-elute” mode, wherein the desired molecules interact with the stationary phase until certain conditions are created that cause the desired molecules to release from the stationary phase and elute from the chromatography resin; or in a “pass through” mode, wherein one or more impurities present in the mobile phase, but not the desired molecules, interact with the chromatography resin, allowing the desired molecules to “pass through” the chromatography resin, while the impurities remain associated with the chromatography resin. In some aspects, the chromatography resin comprises an anion exchange (AEX) resin, a cation exchange (CEX) resin, a pseudo affinity chromatography resin, a hydrophobic interaction resin, a hydrophobic charge induction chromatography resin, a mixed mode resin, an immobilized metal affinity resin, a ceramic hydroxyapatite resin, a fluoro hydroxyapatite resin, a ceramic fluoroapatite, and any combination thereof. In some aspects, the chromatography resin comprises a mixed-mode chromatography (MMC) resin.

As used herein, the term “extracellular vesicle” or “EV” refers to a cell-derived vesicle comprising a membrane that encloses an internal space. Extracellular vesicles comprise all membrane-bound vesicles (e.g., exosomes, microvesicles, microsomes, extracellular bodies, apoptotic bodies, and/or nanovesicles) that have a smaller diameter than the cell from which they are derived. In some aspects, extracellular vesicles comprise a population of exosomes and/or microvesicles. In some aspects, extracellular vesicles range in diameter from 20 nm to 1000 nm, and can comprise various macromolecular molecules either within the internal space (i.e., lumen), displayed on the external surface and/or the luminal surface of the EV, and/or spanning the membrane. In some aspects, the molecules in the EVs can comprise nucleic acids, proteins, carbohydrates, lipids, small molecules, and/or combinations thereof. In certain aspects, an EV comprises a scaffold moiety. By way of example and without limitation, EVs include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). EVs can be derived from a living or dead organism, explanted tissues or organs, prokaryotic or eukaryotic cells, and/or cultured cells. In some aspects, the EVs are produced by cells that express one or more transgene products. The EVs that can be purified by the present methods include exosomes, microsomes, microvesicles, extracellular bodies, apoptotic bodies, nanovesicles, or any combination thereof.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome with the plasma membrane. As described infra, an exosome can be derived from a producer cell, and isolated from the producer cell based on its size, density, biochemical parameters, or a combination thereof.

In some aspects, the exosome of the present disclosure is engineered by associating (e.g., linking, e.g., covalently linking) at least one moiety, e.g., payload, e.g., a biologically active molecule (e.g., a protein such as an antibody or ADC, a RNA or DNA such as an antisense oligonucleotide, a small molecule drug, a toxin, a STING agonist, or PROTAC) to the exosome, directly or indirectly, e.g., via a linker, a scaffold moiety, or any combination thereof.

As used herein, the term “payload” refers to an agent that acts on a target (e.g., a target cell) that is contacted with the EV (e.g., exosome). In some aspects, unless indicated otherwise, the term payload can be used interchangeably with the term “biologically active molecule.” Non-limiting examples of payload that can be included on the EV, e.g., exosome, are polypeptides (e.g., an antibody, an antigen, an adjuvant, a ligand, a receptor, an immune modulator, and or any fragment thereof), a polynucleotide, a viral particle, a small molecule, or any combination thereof. Payloads that can be introduced into an EV, e.g., exosome, and/or a producer cell include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, siRNA, antisense oligonucleotide, a phosphorodiamidate morpholino oligomer (PMO), a peptide-conjugated phosphorodiamidate morpholino oligomer (PPMO), or combinations thereof), amino acids (e.g., amino acids comprising a detectable moiety or a toxin or that disrupt translation), polypeptides (e.g., enzymes), lipids, carbohydrates, and small molecules (e.g., small molecule drugs and toxins). In certain aspects, a payload comprises an antigen.

In some aspects, the payload is a protein, a peptide, a glycolipid, or a glycoprotein.

In certain aspects, the payload is a polynucleotide. In some of these aspects, the polynucleotide includes, but is not limited to, an mRNA, a miRNA, an siRNA, an antisense oligonucleotide (e.g., antisense RNA or antisense DNA), a phosphorodiamidate morpholino oligomer (PMO), a peptide-conjugated phosphorodiamidate morpholino oligomer (PPMO), an shRNA, a lncRNA, a dsDNA, and combinations thereof. In some aspects, the polynucleotide is an RNA (e.g., an mRNA, a miRNA, an siRNA, an antisense oligonucleotide (e.g., antisense RNA), an shRNA, or an lncRNA). In some aspects, the polynucleotide can target a transcription factor. In some of these aspects, when the polynucleotide is an mRNA, it can be translated into a desired polypeptide. In some aspects, the polynucleotide is a microRNA (miRNA) or pre-miRNA molecule. In some of these aspects, the miRNA is delivered to the cytoplasm of the target cell, such that the miRNA molecule can silence a native mRNA in the target cell. In some aspects, the polynucleotide is a small interfering RNA (siRNA) or a short hairpin RNA (shRNA) capable of interfering with the expression of an oncogene or other dysregulating polypeptides. In some of these aspects, the siRNA is delivered to the cytoplasm of the target cell, such that the siRNA molecule can silence a native mRNA in the target cell. In some aspects, the polynucleotide is an antisense oligonucleotide (e.g., antisense RNA) that is complementary to an mRNA. In some aspects, the polynucleotide is a long non-coding RNA (lncRNA) capable of regulating gene expression and modulating diseases. In some aspects, the polynucleotide is a DNA that can be transcribed into an RNA. In some of these aspects, the transcribed RNA can be translated into a desired polypeptide.

As used herein, the term “residual nucleic acid molecule” or “residual nucleic acid molecules” refers to contaminating nucleic acids and/or polynucleotides present in a sample comprising an EV, e.g., an exosome. During the preparation of EVs undesired nucleic acid molecules can be present in solution with the EVs. In some aspects, the residual nucleic acid molecules comprise antisense oligomers that failed to associate with the EVs. In some aspects, the residual nucleic acid molecules comprise DNA and/or RNA released by cells during the EV manufacturing process.

As used herein, the term “nanovesicle” refers to an extracellular vesicle with a diameter between 20-250 nm (e.g., between 30-150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. Nanovesicles, once derived from a producer cell, can be isolated from the producer cell based on its size, density, biochemical parameters, or a combination thereof. EVs can be derived from a living or dead organism, explanted tissues or organs, prokaryotic or eukaryotic cells, and/or cultured cells.

The term “microvesicle” or “microparticle,” as used herein, is a type of EV, which is between 50 and 1,000 nanometers (nm) in diameter, and which is found in many types of body fluids as well as the interstitial space between cells. Microvesicles are membrane-bound vesicles containing phospholipids, ranging from 100 nm to 1000 nm shed from almost all cell types. Microvesicles play a role in intercellular communication and can transport mRNA, miRNA, and proteins between cells. They originate directly from the plasma membrane of the cell and reflect the antigenic content of the cells from which they originate. They remove misfolded proteins, cytotoxic agents and metabolic waste from the cell.

The term “microsome,” as used herein, refers to heterogeneous vesicle-like artifacts (~20-200 nm diameter) re-formed from pieces of the endoplasmic reticulum (ER) when eukaryotic cells are broken-up in the laboratory; microsomes are not present in healthy, living cells. Microsomes can be concentrated and separated from other cellular debris by differential centrifugation. Unbroken cells, nuclei, and mitochondria sediment out at 10,000 g, whereas soluble enzymes and fragmented ER, which contains cytochrome P450 (CYP), remain in solution (g is the Earth's gravitational acceleration). Microsomes have a reddish-brown color, due to the presence of the heme.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by about 10%, about 20%, about 30%, about 40%, about 50%, about 60%, about 70%, about 80%, about 90%, about 95%, about 96%, about 97%, about 98%, about 99%, about 99.9%, about 99.99%, about 99.999%, about 99.9999%, or greater than about 99.9999% compared to the starting material. In some aspects, isolated EV preparations according to the present disclosure are substantially free of residual contaminating products, including residual biologic products. In some aspects, the isolated EV preparations according to the present disclosure are 100% free, about 99% free, about 98% free, about 97% free, about 96% free, about 95% free, about 94% free, about 93% free, about 92% free, about 91% free, or about 90% free of any contaminating biological matter. Residual contaminating products can include abiotic materials (including chemicals) or unwanted nucleic acids, proteins, lipids, or metabolites. Substantially free of residual biological products can also mean that the EV composition contains no detectable producer cells and that only EVs are detectable.

The term “excipient” refers to an inert substance added to assist in the purification of the EVs. Excipients can modulate the structure of the EV, modulate the adsorption rate of the EVs or the impurities, alter the polarity of the solution being purified, and perform other functions to provide an increase in the purity of the EVs.

As used herein, the term “substantially free” means that a purified composition comprising EVs comprise less than about 10% (m/v) of macromolecules by mass/volume percentage concentration. Some fractions may contain less than about 0.001%, less than about 0.01%, less than about 0.05%, less than about 0.1%, less than about 0.2%, less than about 0.3%, less than about 0.4%, less than about 0.5%, less than about 0.6%, less than about 0.7%, less than about 0.8%, less than about 0.9%, less than about 1%, less than about 2%, less than about 3%, less than about 4%, less than about 5%, less than about 6%, less than about 7%, less than about 8%, less than about 9%, or less than about 10% (m/v) of macromolecules.

As used herein, the term “macromolecule” means a molecule containing a very large number of atoms, such as nucleic acids, proteins, lipids, carbohydrates, metabolites, and/or a combination thereof. In some aspects, “macromolecules” are part of impurities that can be removed during purification as described herein.

The term “nucleic acid molecule” refers to any nucleotide or nucleoside or any polymer or analog thereof, including but not limited to deoxyribonucleic acid (DNA) molecules, ribonucleic acid (RNA) molecules, peptide nucleic acid molecules, locked nucleic acid (LNA) molecules, morpholino nucleic acid molecules, glycol nucleic acid molecules, threose nucleic acid molecules, and any polymers, analogs, or combinations thereof. The term “polynucleotide,” as used herein, refers to a nucleic acid molecule comprising at least two individual nucleotide units.

The term “nuclease” as used herein refers to a protein, e.g., an enzyme that is capable of catalyzing the cleavage of a nucleic acid molecule. In some aspects, the nuclease is an “endonuclease,” which refers to a nuclease that catalyzes cleavage of a nucleic acid molecule between two adjacent nucleotides, wherein at neither of the adjacent nucleotides are at the terminus of the nucleic acid molecule, e.g. an endonuclease catalyzes cleavage between the 5′ and 3′ end of a nucleic acid molecule. Conversely, in some aspects, the nuclease comprises an “exonuclease,” which catalyzes the cleavage of a nucleic acid molecule by removing one or more nucleotides at one or both ends of the nucleic acid molecule, e.g., by removing the 5′ or 3′ nucleotide from the nucleic acid molecule. In certain aspects, the nuclease is selected from a salt active nuclease (SAN), a benzonase, a denarase, a kryptonase, and any combination thereof. In some aspects, more than one nuclease is applied to the chromatography resins disclosed herein.

The terms “anion” and “cation” refer to negatively and positively charged ions, respectively. A “divalent” cation refers to a cation with a valence of 2+. Examples of divalent cations include, but are not limited to, Ca²⁺, Mg²⁺, Co²⁺, Ni²⁺, Zn²⁺, Ba²⁺, Sr²⁺, Al²⁺, Ag²⁺, Cu²⁺, and Mn²⁺. A “monovalent cation refers to a cation with a valence of 1+. Examples of monovalent cations include, but are not limited to, Li⁺, K⁺, Na⁺, NH4⁺, Cu⁺. Examples of anions include, but are not limited to, SCN⁻, Cl⁻, SO4⁻, and PO₄. In some aspects, an anion and/or a cation (e.g., monovalent cation or divalent cation) can be present in a salt, e.g., a mixture of at least one anion and at least one cation of complementary valences. In some aspects, the salt is selected from MgCl₂, Mg(SCN)₂, Mg(SO₄)₂, Mg(PO₄)₂, and any combination thereof.

The terms “polypeptide,” “peptide,” and “protein” are used interchangeably herein to refer to polymers of amino acids, e.g, proteins and/or peptides, of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. In some aspects of the present disclosure, the biologically active molecule attached to the EV is a polypeptide, e.g., an antibody or an antigen binding portion thereof, a fusion protein, a cytokine, or an enzyme.

Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multi-chain polypeptides. Most commonly, disulfide linkages are found in multi-chain polypeptides. The term polypeptide can also apply to amino acid polymers in which one or more amino acid residues are an artificial chemical analog of a corresponding naturally occurring amino acid. In some aspects, a “peptide” can be less than or equal to 50 amino acids long, e.g., about 5, 10, 15, 20, 25, 30, 35, 40, 45, or 50 amino acids long.

A “recombinant” polypeptide or protein refers to a polypeptide or protein produced via recombinant DNA technology. Recombinantly produced polypeptides and proteins expressed in engineered host cells are considered isolated for the purpose of the disclosure, as are native or recombinant polypeptides, which have been separated, fractionated, or partially or substantially purified by any suitable technique. The polypeptides disclosed herein can be recombinantly produced using methods known in the art. Alternatively, the proteins and peptides disclosed herein can be chemically synthesized. In some aspects of the present disclosure, the Scaffold X and/or Scaffold Y proteins present in EVs are recombinantly produced by overexpressing the scaffold proteins in the producer cells, so that levels of scaffold proteins in the resulting EVs are significantly increased with respect to the levels of scaffold proteins present in EVs of producer cells not overexpressing such scaffold proteins.

As used herein, the term “scaffold moiety” refers to a molecule, e.g., a protein such as Scaffold X or Scaffold Y, that can be used to anchor a molecule, e.g., a biologically active molecule, to the EV either on the luminal surface or on the exterior surface of the EV. In certain aspects, a scaffold moiety comprises a synthetic molecule. In some aspects, a scaffold moiety comprises a non-polypeptide moiety. In other aspects, a scaffold moiety comprises, e.g., a lipid, carbohydrate, protein, or combination thereof (e.g., a glycoprotein or a proteolipid) that naturally exists in the EV. In some aspects, a scaffold moiety comprises a lipid, carbohydrate, or protein that does not naturally exist in the EV. In some aspects, a scaffold moiety comprises a lipid or carbohydrate, which naturally exists in the EV but has been enriched in the EV with respect to basal/native/wild type levels. In some aspects, a scaffold moiety comprises a protein which naturally exists in the EV but has been engineered to be enriched in the EV, e.g., by recombinant overexpression in the producer cell, with respect to basal/native/wild type levels. In certain aspects, a scaffold moiety is Scaffold X. In some aspects, a scaffold moiety is Scaffold Y. In further aspects, a scaffold moiety comprises both Scaffold X and Scaffold Y.

As used herein, the term “Scaffold X” or “PrX” refers to EV proteins that have been identified on the surface of EVs. See, e.g., U.S. Pat. No. 10,195,290, which is incorporated herein by reference in its entirety. Non-limiting examples of Scaffold X proteins include: prostaglandin F2 receptor negative regulator (“PTGFRN”); basigin (“BSG”); immunoglobulin superfamily member 2 (“IGSF2”); immunoglobulin superfamily member 3 (“IGSF3”); immunoglobulin superfamily member 8 (“IGSF8”); integrin beta-1 (“ITGB1”); integrin alpha-4 (“ITGA4”); 4F2 cell-surface antigen heavy chain (“SLC3A2”); and a class of ATP transporter proteins (“ATP1A1,” “ATP1A2,” “ATP1A3,” “ATP1A4,” “ATP1B3,” “ATP2B1,” “ATP2B2,” “ATP2B3,” “ATP2B”). In some aspects, a Scaffold X protein can be a whole protein or a fragment thereof (e.g., functional fragment, e.g., the smallest fragment that is capable of anchoring another moiety on the exterior surface or on the luminal surface of the EV). In some aspects, a Scaffold X can anchor a moiety, e.g., a biologically active molecule to the external surface or the luminal surface of the EV. Non-limiting examples of other Scaffold X proteins include e.g., CD13 (aminopeptidase N), MME (membrane metalloendopeptidase), ENPP1 (ectonucleotide pyrophosphatase/phosphodiesterase family member 1), NRP1 (neuropilin-1), CD9, CD63, CD81, PDGFR, GPI anchor proteins, lactadherin, LAMP2, and LAMP2B.

As used herein, the term “Scaffold Y” refers to EV proteins that have been identified within the lumen of EVs. See, e.g., International Publ. Nos. WO/2019/099942 and WO 2020/101740, each of which is incorporated herein by reference in its entirety. Non-limiting examples of Scaffold Y proteins include: myristoylated alanine rich Protein Kinase C substrate (“MARCKS”); myristoylated alanine rich Protein Kinase C substrate like 1 (“MARCKSL1”); and brain acid soluble protein 1 (“BASP1”). In some aspects, a Scaffold Y protein can be a whole protein or a fragment thereof (e.g., functional fragment, e.g., the smallest fragment that is capable of anchoring a moiety on the luminal surface of the EV). In some aspects, a Scaffold Y can anchor a moiety on the luminal surface of the EV. In some aspects of the present disclosure, a moiety can be covalently attached to a Scaffold Y. In some aspects, the moiety can be attached to Scaffold Y on the luminal surface of the EV.

As used herein the term “surface-engineered EV” (e.g., Scaffold X-engineered EV) refers to an EV with the membrane or the surface of the EV modified in its composition so that the surface of the engineered EV is different from that of the EV prior to the modification or of the naturally occurring EV. The engineering can be on the surface of the EV or in the membrane of the EV so that the exterior surface of the EV is changed. For example, the membrane can be modified in its composition of, e.g., a protein, a lipid, a small molecule, a carbohydrate, or a combination thereof. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV comprises an exogenous protein (i.e., a protein that the EV does not naturally express) or a fragment or variant thereof that can be exposed to the surface of the EV or can be an anchoring point (attachment) for a moiety exposed on the exterior surface of the EV. In other aspects, a surface-engineered EV comprises a higher expression (e.g., higher number) of a natural EV protein (e.g., Scaffold X) or a fragment or variant thereof that can be exposed to the surface of the EV or is capable of being an anchoring point (attachment) for a moiety exposed on the surface of the EV.

As used herein the term “lumen-engineered exosome” (e.g., Scaffold Y-engineered exosome) refers to an exosome with the membrane or the lumen of the exosome modified in its composition so that the lumen of the engineered exosome is different from that of the exosome prior to the modification or of the naturally occurring exosome. The engineering can be directly on the luminal surface or in the membrane of the exosome so that the lumen of the exosome is changed. For example, the membrane is modified in its composition of a protein, a lipid, a small molecule, a carbohydrate, etc. so that the lumen of the exosome is modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some aspects, a lumen-engineered exosome comprises an exogenous protein (i.e., a protein that the exosome does not naturally express) or a fragment or variant thereof that can be exposed on the luminal surface of the exosome or can be an anchoring point (attachment) for a moiety exposed on the inner layer of the exosome. In other aspects, a lumen-engineered exosome comprises a higher expression of a natural exosome protein (e.g., Scaffold X or Scaffold Y) or a fragment or variant thereof that can be exposed to the lumen of the exosome or can be an anchoring point (attachment) for a moiety exposed on the luminal surface of the exosome.

As used herein the term “linked to,” “fused,” or “conjugated to” are used interchangeably and refer to a covalent or non-covalent bond formed between a first moiety and a second moiety, e.g., Scaffold X and an antigen, e.g., a scaffold moiety expressed in or on the extracellular vesicle and an antigen, e.g., Scaffold X (e.g., a PTGFRN protein), respectively, in the luminal surface of or on the external surface of the extracellular vesicle. In some aspects, a payload disclosed herein can be directly linked to the exterior surface and/or the luminal surface of an EV (e.g., exosome). As used herein, the term “directly linked,” “directly fused,” or “directly conjugated to” refer to the process of linking (fusing or conjugating) a moiety (e.g., a payload and/or targeting moiety) to the surface of an EV (e.g., exosome) without the use of a scaffold moiety disclosed herein.

As used herein, the term “fusion protein” refers to two or more proteins that are linked or conjugated to each other. For instance, in some aspects, a fusion protein that can be expressed in an EV (e.g., exosome) disclosed herein comprises (i) a payload (e.g., antigen, adjuvant, and/or immune modulator) and (ii) a scaffold moiety (e.g., Scaffold X and/or Scaffold Y). In some aspects, a fusion protein that can be expressed in an EV (e.g., exosome) useful for the present disclosure comprises (i) a targeting moiety and (ii) a scaffold moiety (e.g., Scaffold X and/or Scaffold Y). As described herein, in some aspects, EVs (e.g., exosomes) of the present disclosure can express multiple fusion proteins, wherein a first fusion protein comprises (i) a payload (e.g., antigen, adjuvant, and/or immune modulator) and (ii) a scaffold moiety (e.g., Scaffold X and/or Scaffold Y), and wherein a second fusion protein comprises (i) a targeting moiety and (ii) a scaffold moiety (e.g., Scaffold X and/or Scaffold Y).

### II. Methods of the Present Disclosure

Certain aspects of the present disclosure relate to isolation, purification and/or sub-fractionation of EVs by chromatographic purification methods. Certain aspects of the present disclosure are directed to methods of preparing purified extracellular vesicles (EVs) from a sample comprising EVs and one or more nucleic acid molecules, comprising: contacting a chromatography resin associated with the sample with a wash buffer; wherein the wash buffer comprises MgCl₂, CaCl₂, KCl, and/or NaCl and does not comprise a nuclease. Some aspects of the present disclosure are directed to methods of reducing the concentration of residual nucleic acid molecule in a sample comprising extracellular vesicles (EVs), comprising contacting a chromatography resin associated with the sample with a wash buffer; wherein the wash buffer comprises MgCl₂, CaCl₂, KCl, and/or NaCl and does not comprise a nuclease.

In some aspects, the sample does not come into a contact with a nuclease during the method. Use of an endonuclease is the gold standard for reducing or removing nucleic acid molecules from a sample. However, nuclease treatment and its subsequent removal add time to the manufacturing process, and it can be difficult to ensure that no residual nuclease remains. The present disclosure provides novel methods of reducing or eliminating contaminating nucleic acid molecules from a sample without the use of a nuclease. In some aspects, the contaminating nucleic acid molecules are removed by washing the sample (i.e., associated with a chromatography resin) with a buffer comprising MgCl₂, CaCl₂, KCl, and/or NaCl.

Some aspects of the present disclosure are directed to a method of preparing purified extracellular vesicles (EVs) from a sample comprising EVs and one or more nucleic acid molecules, comprising: (i) contacting the sample with a chromatography resin and (ii) contacting the chromatography resin associated with the sample with a wash buffer comprising MgCl₂, CaCl₂, KCl, and/or NaCl; wherein the wash buffer does not comprise a nuclease; and wherein the (ii) washing follows the (i) contacting.

Some aspects of the present disclosure are directed to a method of reducing the concentration of residual nucleic acid molecule in a sample comprising extracellular vesicles (EVs), comprising (i) contacting the sample with a chromatography resin and (ii) contacting the chromatography resin associated with the sample with a wash buffer comprising MgCl₂, CaCl₂, KCl, and/or NaCl; wherein the wash buffer does not comprise a nuclease; and wherein the (ii) washing follows the (i) contacting.

Some aspects of the present disclosure are directed to a method of preparing purified extracellular vesicles (EVs) from a sample comprising EVs and one or more nucleic acid molecules, comprising: (i) contacting the sample with a chromatography resin; (ii) contacting the chromatography resin associated with the sample with a wash buffer comprising MgCl₂, CaCl₂, KCl, and/or NaCl, wherein the wash buffer does not comprise a nuclease; and (iii) eluting an eluent from the chromatography resin, wherein the eluent comprises the EVs, e.g., exosomes.

In some aspects, the sample comprising EVs, e.g., exosomes, and one or more nucleic acid molecules is contacted with the chromatography resin in a loading buffer. In some aspects, the sample comprises the EVs, e.g., exosomes, and the one or more nucleic acid molecules in a loading buffer.

In some aspects, the flow through following the wash buffer comprises fragments of the one or more nucleic acid molecules. In some aspects, the flow through following the second wash buffer comprises fragments of the one or more nucleic acid molecules.

In some aspects, one or more of the sample, the wash buffer, the second wash buffer, and the elution buffer is allowed to flow through the chromatography resin by unassisted gravitational flow. In some aspects, one or more of the sample, the wash buffer, the second wash buffer, and the elution buffer is pumped, i.e., by positive pressure applied to a loading site of the chromatography resin or by negative pressure applied at a flow-through site of the chromatography resin, across the chromatography resin at an optimal flow rate. In some aspects, the flow rate is from at least about 0.01 membrane volumes per minute (MV/min) to at least about 5 MV/min, at least about 0.1 MV/min to at least about 5 MV/min, at least about 0.5 MV/min to at least about 5 MV/min, at least about 1 MV/min to at least about 5 MV/min, at least about 0.01 MV/min to at least about 4 MV/min, at least about 0.01 MV/min to at least about 3 MV/min, at least about 0.01 MV/min to at least about 2 MV/min, at least about 0.01 MV/min to at least about 1 MV/min, or at least about 0.01 MV/min to at least about 0.1 MV/min. In some aspects, the flow rate is at least about 0.01 MV/min, at least about 0.05 MV/min, at least about 0.1 MV/min, at least about 0.5 MV/min, at least about 1 MV/min, at least about 2 MV/min, at least about 3 MV/min, at least about 4 MV/min, or at least about 5 MV/min. In certain aspects, the follow rate is at least about 0.1 MV/min. In some aspects, the flow rate during the wash step is from at least about 0.01 MV/min to at least about 1 MV/min. In some aspects, the flow rate during the wash step is at least about 0.1 MV/min.

The methods disclosed herein reduce the level, e.g., concentration, of one or more nucleic acid molecules in a sample comprising EVs, e.g., exosomes, and one or more nucleic acid molecules. In some aspects, the sample contacted with the chromatography resin comprises a starting concentration of the one or more nucleic acid molecules, and the eluent comprises an eluted concentration of the one or more nucleic acid molecules, wherein the eluted concentration of the one or more nucleic acid molecules is less than about 10%, less than about 5%, less than about 1%, less than about 0.5%, less than about 0.1%, less than about 0.05%, less than about 0.01%, or less than about 0.001% that of the starting concentration of the one or more nucleic acid molecules.

In some aspects, the reduction in residual nucleic acid molecules is at least about 1000-fold to at least about 100,000-fold. In some aspects, the reduction in residual nucleic acid molecules is at least about 1000-fold. In some aspects, the reduction in residual nucleic acid molecules is at least about 5000-fold. In some aspects, the reduction in residual nucleic acid molecules is at least about 10,000-fold. In some aspects, the reduction in residual nucleic acid molecules is at least about 50,000-fold. In some aspects, the reduction in residual nucleic acid molecules is at least about 100,000-fold.

In some aspects, the eluted concentration of the one or more nucleic acid molecules is less than about 10% that of the starting concentration of the one or more nucleic acid molecules. In some aspects, the eluted concentration of the one or more nucleic acid molecules is less than about 5% that of the starting concentration of the one or more nucleic acid molecules. In some aspects, the eluted concentration of the one or more nucleic acid molecules is less than about 4% that of the starting concentration of the one or more nucleic acid molecules. In some aspects, the eluted concentration of the one or more nucleic acid molecules is less than about 3% that of the starting concentration of the one or more nucleic acid molecules. In some aspects, the eluted concentration of the one or more nucleic acid molecules is less than about 2% that of the starting concentration of the one or more nucleic acid molecules. In some aspects, the eluted concentration of the one or more nucleic acid molecules is less than about 1% that of the starting concentration of the one or more nucleic acid molecules. In some aspects, the eluted concentration of the one or more nucleic acid molecules is less than about 0.5% that of the starting concentration of the one or more nucleic acid molecules. In some aspects, the eluted concentration of the one or more nucleic acid molecules is less than about 0.1% that of the starting concentration of the one or more nucleic acid molecules. In some aspects, the eluted concentration of the one or more nucleic acid molecules is less than about 0.05% that of the starting concentration of the one or more nucleic acid molecules. In certain aspects, the eluted sample comprises no detectable nucleic acid molecules.

### II.A. Wash Buffer

Certain aspects of the present disclosure are directed to a method comprising contacting a chromatography resin associated with a sample with a wash buffer comprising MgCl₂, CaCl₂, KCl, and/or NaCl, wherein the wash buffer does not comprise a nuclease. In some aspects, the Cl of the MgCl₂, CaCl₂, KCl, and/or NaCl can be replaced by a Br, e.g., in some aspects the buffer comprises MgBr₂, CaBr₂, KBr, and/or NaBr.

In some aspects, the chromatography resin associated with the sample is contacted with the wash buffer, e.g., the wash buffer comprising MgCl₂, CaCl₂, KCl, and/or NaCl, one time. In some aspects, the chromatography resin is contacted with the wash buffer, e.g., the wash buffer comprising MgCl₂, CaCl₂, KCl, and/or NaCl, at least two times, e.g., the method comprises contacting the chromatography resin with a wash buffer, e.g., a wash buffer comprising MgCl₂, CaCl₂, KCl, and/or NaCl, allowing the wash buffer to pass through the chromatography resins, and then contacting the chromatography resin with the wash buffer, e.g., the wash buffer comprising MgCl₂, CaCl₂, KCl, and/or NaCl, a second time. In some aspects, the chromatography resin is contacted with the wash buffer, e.g., the wash buffer comprising MgCl₂, CaCl₂, KCl, and/or NaCl, at least three times. In some aspects, the chromatography resin is contacted with the wash buffer, e.g., the wash buffer comprising MgCl₂, CaCl₂, KCl, and/or NaCl, at least four times. In some aspects, the chromatography resin is contacted with the wash buffer, e.g., the wash buffer comprising MgCl₂, CaCl₂, KCl, and/or NaCl, at least five times. In some aspects, the wash buffer, e.g., the wash buffer comprising MgCl₂, CaCl₂, KCl, and/or NaCl, of each contacting is the same. In some aspects, the wash buffer, e.g., the wash buffer comprising MgCl₂, CaCl₂, KCl, and/or NaCl, of each contacting is different.

In some aspects, the wash buffer, e.g., the wash buffer comprising MgCl₂, CaCl₂, KCl, and/or NaCl, is contacted with the chromatography resin, and the flow through is blocked, wherein the wash buffer, e.g., the wash buffer comprising MgCl₂, CaCl₂, KCl, and/or NaCl, remains in contact with the chromatography resin for a period of time. In some aspects, the wash buffer, e.g., the wash buffer comprising MgCl₂, CaCl₂, KCl, and/or NaCl, remains in contact with the chromatography resin for at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 60 minutes, at least about 75 minutes, at least about 90 minutes, at least about 105 minutes, at least about 120 minutes, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 15 hours, at least about 18 hours, at least about 21 hours, or at least about 24 hours.

In some aspects, the wash buffer, e.g., the wash buffer comprising MgCl₂, CaCl₂, KCl, and/or NaCl, is contacted with the chromatography resin, and the flow through is collected and contacted again with the chromatography resin. In some aspects, the flow through is circulated back to contact the chromatography resin a second time.

### II.A.1. Salts

Some aspects of the present disclosure are directed to methods comprising contacting a chromatography resin associated with the sample with a wash buffer; wherein the wash buffer comprises MgCl₂, CaCl₂, KCl, and/or NaCl and does not comprise a nuclease. In some aspects, the wash buffer comprises Mg²⁺, e.g., MgCl₂ or MgBr₂. In some aspects, the wash buffer comprises Ca²⁺, e.g., CaCl₂ or CaBr₂. In some aspects, a salt comprising a cation other than Mg²⁺ or Ca²⁺ can be used in the wash. In some aspects, the cation can comprise a monovalent cation. In some aspects, the monovalent cation is selected from Li⁺, K⁺, Na⁺, NH4⁺, Cu⁺, and any combination thereof. In some aspects, the wash buffer comprises Na⁺, e.g., NaCl. In some aspects, the cation can comprises a divalent cation. In some aspects, the divalent cation is selected from Co²⁺, Ni²⁺, Zn²⁺, Ba²⁺, Sr²⁺, Al²⁺, Ag²⁺, Cu²⁺, Mn²⁺, and any combination thereof.

In some aspects, the cation is associated with an anion, e.g., in a salt. In some aspects, the nuclease wash buffer comprises a salt, wherein the salt comprises a cation disclosed herein and an anion. In some aspects, the salt comprises a cation disclosed herein and an anion selected from SCN⁻, Cl⁻, Br⁻, SO₄⁻, PO₄⁻, and any combination thereof. In some aspects, the salt comprises a cation disclosed herein and Cl⁻. In some aspects, the salt comprises a cation disclosed herein and Br. In some aspects, the salt comprises a cation disclosed herein and SCN⁻. In some aspects, the salt comprises a cation disclosed herein and SO₄⁻. In some aspects, the salt comprises a cation disclosed herein and PO₄⁻.

In some aspects, the wash buffer comprises MgCl₂, MgBr₂, Mg(SCN)₂, Mg(SO₄)₂, Mg(PO₄)₂, or any combination thereof. In some aspects, the wash buffer comprises MgCl₂. In some aspects, the wash buffer comprises MgBr₂. In some aspects, the wash buffer comprises Mg(SCN)₂. In some aspects, the wash buffer comprises Mg(SO₄)₂. In some aspects, the wash buffer comprises Mg(PO₄)₂.

In some aspects, the wash buffer comprises CaCl₂, CaBr₂, Ca(SCN)₂, Ca(SO₄)₂, Ca(PO₄)₂, or any combination thereof. In some aspects, the wash buffer comprises CaCl₂. In some aspects, the wash buffer comprises CaBr₂. In some aspects, the wash buffer comprises Ca(SCN)₂. In some aspects, the wash buffer comprises Ca(SO₄)₂. In some aspects, the wash buffer comprises Ca(PO₄)₂.

In some aspects, the wash buffer comprises CoCl₂, CoBr₂, Co(SCN)₂, Co(SO₄)₂, Co(PO₄)₂, or any combination thereof. In some aspects, the wash buffer comprises NiCl₂, NiBr₂, Ni(SCN)₂, Ni(SO₄)₂, Ni(PO₄)₂, or any combination thereof. In some aspects, the wash buffer comprises ZnCl₂, ZnBr₂, Zn(SCN)₂, Zn(SO₄)₂, Zn(PO₄)₂, or any combination thereof. In some aspects, the wash comprises BaCl2, BaBr₂, Ba(SCN)₂, Ba(SO₄)₂, Ba(PO₄)₂, or any combination thereof. In some aspects, the wash buffer comprises SrCl2, SrBr₂, Sr(SCN)₂, Sr(SO₄)₂, Sr(PO₄)₂, or any combination thereof. In some aspects, the wash buffer comprises AlCl₂, AlBr₂, Al(SCN)₂, Al(SO₄)₂, Al(PO₄)₂, or any combination thereof. In some aspects, the wash buffer comprises AgCl₂, AgBr₂, Ag(SCN)₂, Ag(SO₄)₂, Ag(PO₄)₂, or any combination thereof. In some aspects, the wash buffer comprises CuCl₂, CuBr₂, Cu(SCN)₂, Cu(SO₄)₂, Cu(PO₄)₂, or any combination thereof. In some aspects, the wash buffer comprises MnCl₂, MnBr₂, Mn(SCN)₂, Mn(SO₄)₂, Mn(PO₄)₂, or any combination thereof.

In some aspects, the wash buffer comprises at least about 1 mM to at least about 1.0 M of the cation, e.g., Mg²⁺, Ca²⁺, Na⁺, or K⁺. In some aspects, the wash buffer comprises at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 20 mM, at least about 30 mM, at least about 40 mM, at least about 50 mM, at least about 60 mM, at least about 70 mM, at least about 80 mM, at least about 90 mM, at least about 100 mM, at least about 150 mM, at least about 200 mM, at least about 210 mM, at least about 220 mM, at least about 225 mM, at least about 230 mM, at least about 240 mM, at least about 250 mM, at least about 260 mM, at least about 270 mM, at least about 275 mM, at least about 280 mM, at least about 290 mM, at least about 300 mM, at least about 310 mM, at least about 320 mM, at least about 325 mM, at least about 330 mM, at least about 340 mM, at least about 350 mM, at least about 360 mM, at least about 370 mM, at least about 375 mM, at least about 380 mM, at least about 390 mM, at least about 400 mM, at least about 410 mM, at least about 420 mM, at least about 425 mM, at least about 430 mM, at least about 440 mM, at least about 450 mM, at least about 460 mM, at least about 470 mM, at least about 475 mM, at least about 480 mM, at least about 490 mM, at least about 500 mM, at least about 525 mM, at least about 550 mM, at least about 575 mM, at least about 600 mM, at least about 650 mM, at least about 700 mM, at least about 750 mM, at least about 800 mM, at least about 850 mM, at least about 900 mM, at least about 950 mM, or at least about 1000 mM of the cation, e.g., Mg²⁺, Ca²⁺ Na⁺, or K⁺.

In certain aspects, the cation comprises Mg²⁺, e.g., MgCl₂ or MgBr₂, and the concentration of the Mg²⁺ in the wash buffer is at least about 200 mM to about 500 mM. In some aspects, the concentration of the Mg²⁺ in the wash buffer is at least about 100 mM, at least about 150 mM, at least about 200 mM, at least about 210 mM, at least about 220 mM, at least about 225 mM, at least about 230 mM, at least about 240 mM, at least about 250 mM, at least about 260 mM, at least about 270 mM, at least about 275 mM, at least about 280 mM, at least about 290 mM, at least about 300 mM, at least about 310 mM, at least about 320 mM, at least about 325 mM, at least about 330 mM, at least about 340 mM, at least about 350 mM, at least about 360 mM, at least about 370 mM, at least about 375 mM, at least about 380 mM, at least about 390 mM, at least about 400 mM, at least about 410 mM, at least about 420 mM, at least about 425 mM, at least about 430 mM, at least about 440 mM, at least about 450 mM, at least about 460 mM, at least about 470 mM, at least about 475 mM, at least about 480 mM, at least about 490 mM, at least about 500 mM, at least about 525 mM, at least about 550 mM, at least about 575 mM, or at least about 600 mM. In some aspects, the concentration of Mg²⁺ in the wash buffer is at least about 200 mM. In some aspects, the concentration of Mg²⁺ in the wash buffer is at least about 225 mM. In some aspects, the concentration of Mg²⁺ in the wash buffer is at least about 250 mM. In some aspects, the concentration of Mg²⁺ in the wash buffer is at least about 275 mM. In some aspects, the concentration of Mg²⁺ in the wash buffer is at least about 300 mM. In some aspects, the concentration of Mg²⁺ in the wash buffer is at least about 325 mM. In some aspects, the concentration of Mg²⁺ in the wash buffer is at least about 350 mM. In some aspects, the concentration of Mg²⁺ in the wash buffer is at least about 375 mM. In some aspects, the concentration of Mg²⁺ in the wash buffer is at least about 400 mM. In some aspects, the concentration of Mg²⁺ in the wash buffer is at least about 425 mM. In some aspects, the concentration of Mg²⁺ in the wash buffer is at least about 450 mM. In some aspects, the concentration of Mg²⁺ in the wash buffer is at least about 475 mM. In some aspects, the concentration of Mg²⁺ in the wash buffer is at least about 500 mM.

In certain aspects, the cation comprises Ca²⁺, e.g., CaCl₂ or CaBr₂, and the concentration of the Ca²⁺ in the wash buffer is at least about 100 mM to about 400 mM. In some aspects, the concentration of the Ca²⁺ in the wash buffer is at least about 50 mM, at least about 60 mM, at least about 70 mM, at least about 80 mM, at least about 90 mM, at least about 100 mM, at least about 150 mM, at least about 200 mM, at least about 210 mM, at least about 220 mM, at least about 225 mM, at least about 230 mM, at least about 240 mM, at least about 250 mM, at least about 260 mM, at least about 270 mM, at least about 275 mM, at least about 280 mM, at least about 290 mM, at least about 300 mM, at least about 310 mM, at least about 320 mM, at least about 325 mM, at least about 330 mM, at least about 340 mM, at least about 350 mM, at least about 360 mM, at least about 370 mM, at least about 375 mM, at least about 380 mM, at least about 390 mM, at least about 400 mM, at least about 410 mM, at least about 420 mM, at least about 425 mM, at least about 430 mM, at least about 440 mM, at least about 450 mM, at least about 460 mM, at least about 470 mM, at least about 475 mM, at least about 480 mM, at least about 490 mM, at least about 500 mM, at least about 525 mM, at least about 550 mM, at least about 575 mM, or at least about 600 mM. In some aspects, the concentration of CA²⁺ in the wash buffer is at least about 100 mM. In some aspects, the concentration of CA²⁺ in the wash buffer is at least about 125 mM. In some aspects, the concentration of CA²⁺ in the wash buffer is at least about 150 mM. In some aspects, the concentration of CA²⁺ in the wash buffer is at least about 175 mM. In some aspects, the concentration of CA²⁺ in the wash buffer is at least about 200 mM. In some aspects, the concentration of Ca²⁺ in the wash buffer is at least about 225 mM. In some aspects, the concentration of Ca²⁺ in the wash buffer is at least about 250 mM. In some aspects, the concentration of Ca²⁺ in the wash buffer is at least about 275 mM. In some aspects, the concentration of Ca²⁺ in the wash buffer is at least about 300 mM. In some aspects, the concentration of Ca²⁺ in the wash buffer is at least about 325 mM. In some aspects, the concentration of Ca²⁺ in the wash buffer is at least about 350 mM. In some aspects, the concentration of Ca²⁺ in the wash buffer is at least about 375 mM. In some aspects, the concentration of Ca²⁺ in the wash buffer is at least about 400 mM.

In certain aspects, the cation comprises Na⁺, and the concentration of the NaCl (or NaBr) in the wash buffer is at least about 600 mM to about 1 M. In some aspects, the concentration of the NaCl (or NaBr) in the wash buffer is at least about 500 mM, at least about 550 mM, at least about 600 mM, at least about 610 mM, at least about 620 mM, at least about 625 mM, at least about 630 mM, at least about 640 mM, at least about 650 mM, at least about 660 mM, at least about 670 mM, at least about 675 mM, at least about 680 mM, at least about 690 mM, at least about 700 mM, at least about 710 mM, at least about 720 mM, at least about 725 mM, at least about 730 mM, at least about 740 mM, at least about 750 mM, at least about 760 mM, at least about 770 mM, at least about 775 mM, at least about 780 mM, at least about 790 mM, at least about 800 mM, at least about 810 mM, at least about 820 mM, at least about 825 mM, at least about 830 mM, at least about 840 mM, at least about 850 mM, at least about 860 mM, at least about 870 mM, at least about 875 mM, at least about 880 mM, at least about 890 mM, at least about 900 mM, at least about 910 mM, at least about 920 mM, at least about 925 mM, at least about 930 mM, at least about 940 mM, at least about 950 mM, at least about 960 mM, at least about 970 mM, at least about 975 mM, at least about 980 mM, at least about 990 mM, or at least about 1 M. In some aspects, the concentration of NaCl (or NaBr) in the wash buffer is at least about 600 mM. In some aspects, the concentration of NaCl (or NaBr) in the wash buffer is at least about 625 mM. In some aspects, the concentration of NaCl (or NaBr) in the wash buffer is at least about 650 mM. In some aspects, the concentration of NaCl (or NaBr) in the wash buffer is at least about 675 mM. In some aspects, the concentration of NaCl (or NaBr) in the wash buffer is at least about 700 mM. In some aspects, the concentration of NaCl (or NaBr) in the wash buffer is at least about 725 mM. In some aspects, the concentration of NaCl (or NaBr) in the wash buffer is at least about 750 mM. In some aspects, the concentration of NaCl (or NaBr) in the wash buffer is at least about 775 mM. In some aspects, the concentration of NaCl (or NaBr) in the wash buffer is at least about 800 mM. In some aspects, the concentration of NaCl (or NaBr) in the wash buffer is at least about 825 mM. In some aspects, the concentration of NaCl (or NaBr) in the wash buffer is at least about 850 mM. In some aspects, the concentration of NaCl (or NaBr) in the wash buffer is at least about 875 mM. In some aspects, the concentration of NaCl (or NaBr) in the wash buffer is at least about 900 mM. In some aspects, the concentration of NaCl (or NaBr) in the wash buffer is at least about 925 mM. In some aspects, the concentration of NaCl (or NaBr) in the wash buffer is at least about 950 mM. In some aspects, the concentration of NaCl (or NaBr) in the wash buffer is at least about 975 mM. In some aspects, the concentration of NaCl (or NaBr) in the wash buffer is at least about 1 M.

In certain aspects, the cation comprises K⁺, and the concentration of the KCl (or KBr) in the wash buffer is at least about 600 mM to about 1 M. In some aspects, the concentration of the KCl (or KBr) in the wash buffer is at least about 500 mM, at least about 550 mM, at least about 600 mM, at least about 610 mM, at least about 620 mM, at least about 625 mM, at least about 630 mM, at least about 640 mM, at least about 650 mM, at least about 660 mM, at least about 670 mM, at least about 675 mM, at least about 680 mM, at least about 690 mM, at least about 700 mM, at least about 710 mM, at least about 720 mM, at least about 725 mM, at least about 730 mM, at least about 740 mM, at least about 750 mM, at least about 760 mM, at least about 770 mM, at least about 775 mM, at least about 780 mM, at least about 790 mM, at least about 800 mM, at least about 810 mM, at least about 820 mM, at least about 825 mM, at least about 830 mM, at least about 840 mM, at least about 850 mM, at least about 860 mM, at least about 870 mM, at least about 875 mM, at least about 880 mM, at least about 890 mM, at least about 900 mM, at least about 910 mM, at least about 920 mM, at least about 925 mM, at least about 930 mM, at least about 940 mM, at least about 950 mM, at least about 960 mM, at least about 970 mM, at least about 975 mM, at least about 980 mM, at least about 990 mM, or at least about 1 M. In some aspects, the concentration of KCl (or KBr) in the wash buffer is at least about 600 mM. In some aspects, the concentration of KCl (or KBr) in the wash buffer is at least about 625 mM. In some aspects, the concentration of KCl (or KBr) in the wash buffer is at least about 650 mM. In some aspects, the concentration of KCl (or KBr) in the wash buffer is at least about 675 mM. In some aspects, the concentration of KCl (or KBr) in the wash buffer is at least about 700 mM. In some aspects, the concentration of KCl (or KBr) in the wash buffer is at least about 725 mM. In some aspects, the concentration of KCl (or KBr) in the wash buffer is at least about 750 mM. In some aspects, the concentration of KCl (or KBr) in the wash buffer is at least about 775 mM. In some aspects, the concentration of KCl (or KBr) in the wash buffer is at least about 800 mM. In some aspects, the concentration of KCl (or KBr) in the wash buffer is at least about 825 mM. In some aspects, the concentration of KCl (or KBr) in the wash buffer is at least about 850 mM. In some aspects, the concentration of KCl (or KBr) in the wash buffer is at least about 875 mM. In some aspects, the concentration of KCl (or KBr) in the wash buffer is at least about 900 mM. In some aspects, the concentration of KCl (or KBr) in the wash buffer is at least about 925 mM. In some aspects, the concentration of KCl (or KBr) in the wash buffer is at least about 950 mM. In some aspects, the concentration of KCl (or KBr) in the wash buffer is at least about 975 mM. In some aspects, the concentration of KCl (or KBr) in the wash buffer is at least about 1 M.

### II.A.2. Second Wash Buffer

In some aspects, the method further comprises contacting the chromatography resin associated with the sample with a second wash buffer, wherein the second wash buffer does not comprise a nuclease. In some aspects, the second wash buffer removes residual cation, e.g., Mg²⁺ or Ca²⁺, from the column. In some aspects, the second wash buffer comprises Mg²⁺ and/or Ca²⁺.

In some aspects, the second wash buffer comprises any wash buffer described herein, e.g., the second wash buffer comprises Mg²⁺ (e.g., MgCl₂ or MgBr₂), Ca²⁺ (e.g., CaCl₂ or CaBr₂), Na⁺ (e.g., NaCl or NaBr), or K⁺ (KCl or KBr).

In some aspects, the second wash buffer comprises NaCl. In some aspects, the second wash buffer comprises at least about 600 mM to about 1 M NaCl. In some aspects, the second wash buffer comprises NaCl at a concentration of at least about 600 mM, at least about 625 mM, at least about 650 mM, at least about 675 mM, at least about 700 mM, at least about 725 mM, at least about 750 mM, at least about 775 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1 M NaCl. In some aspects, the second wash buffer comprises at least about 600 mM NaCl. In some aspects, the second wash buffer comprises at least about 650 mM NaCl. In some aspects, the second wash buffer comprises at least about 700 mM NaCl. In some aspects, the second wash buffer comprises at least about 750 mM NaCl. In some aspects, the second wash buffer comprises at least about 775 mM NaCl. In some aspects, the second wash buffer comprises at least about 800 mM NaCl. In some aspects, the second wash buffer comprises at least about 825 mM NaCl. In some aspects, the second wash buffer comprises at least about 850 mM NaCl. In some aspects, the second wash buffer comprises at least about 875 mM NaCl. In some aspects, the second wash buffer comprises at least about 900 mM NaCl. In some aspects, the second wash buffer comprises at least about 950 mM NaCl. In some aspects, the second wash buffer comprises at least about 1 M NaCl.

In some aspects, (i) the wash buffer comprises MgCl₂, wherein the concentration of Mg²⁺ in the wash buffer is at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, or at least about 500 mM Mg²⁺; and the second wash buffer comprises at least about 600 mM to about 1 M NaCl.

In some aspects, (i) the wash buffer comprises MgCl₂, wherein the concentration of Mg²⁺ in the wash buffer is at least about 300 mM; and the second wash buffer comprises at least about 600 mM to about 1 M NaCl. In some aspects, (i) the wash buffer comprises MgCl₂, wherein the concentration of Mg²⁺ in the wash buffer is at least about 325 mM; and the second wash buffer comprises at least about 600 mM to about 1 M NaCl. In some aspects, (i) the wash buffer comprises MgCl₂, wherein the concentration of Mg²⁺ in the wash buffer is at least about 350 mM; and the second wash buffer comprises at least about 600 mM to about 1 M NaCl. In some aspects, (i) the wash buffer comprises MgCl₂, wherein the concentration of Mg²⁺ in the wash buffer is at least about 375 mM; and the second wash buffer comprises at least about 600 mM to about 1 M NaCl. In some aspects, (i) the wash buffer comprises MgCl₂, wherein the concentration of Mg²⁺ in the wash buffer is at least about 400 mM; and the second wash buffer comprises at least about 600 mM to about 1 M NaCl.

In some aspects, (i) the wash buffer comprises MgCl₂, wherein the concentration of Mg²⁺ in the wash buffer is at least about 300 mM; and the second wash buffer comprises at least about 700 mM NaCl. In some aspects, (i) the wash buffer comprises MgCl₂, wherein the concentration of Mg²⁺ in the wash buffer is at least about 325 mM; and the second wash buffer comprises at least about 700 mM NaCl. In some aspects, (i) the wash buffer comprises MgCl₂, wherein the concentration of Mg²⁺ in the wash buffer is at least about 350 mM; and the second wash buffer comprises at least about 700 mM NaCl. In some aspects, (i) the wash buffer comprises MgCl₂, wherein the concentration of Mg²⁺ in the wash buffer is at least about 375 mM; and the second wash buffer comprises at least about 700 mM NaCl. In some aspects, (i) the wash buffer comprises MgCl₂, wherein the concentration of Mg²⁺ in the wash buffer is at least about 400 mM; and the second wash buffer comprises at least about 700 mM NaCl.

In some aspects, (i) the wash buffer comprises MgCl₂, wherein the concentration of Mg²⁺ in the wash buffer is at least about 300 mM; and the second wash buffer comprises at least about 725 mM NaCl. In some aspects, (i) the wash buffer comprises MgCl₂, wherein the concentration of Mg²⁺ in the wash buffer is at least about 325 mM; and the second wash buffer comprises at least about 725 mM NaCl. In some aspects, (i) the wash buffer comprises MgCl₂, wherein the concentration of Mg²⁺ in the wash buffer is at least about 350 mM; and the second wash buffer comprises at least about 725 mM NaCl. In some aspects, (i) the wash buffer comprises MgCl₂, wherein the concentration of Mg²⁺ in the wash buffer is at least about 375 mM; and the second wash buffer comprises at least about 725 mM NaCl. In some aspects, (i) the wash buffer comprises MgCl₂, wherein the concentration of Mg²⁺ in the wash buffer is at least about 400 mM; and the second wash buffer comprises at least about 725 mM NaCl.

In some aspects, (i) the wash buffer comprises MgCl₂, wherein the concentration of Mg²⁺ in the wash buffer is at least about 300 mM; and the second wash buffer comprises at least about 750 mM NaCl. In some aspects, (i) the wash buffer comprises MgCl₂, wherein the concentration of Mg²⁺ in the wash buffer is at least about 325 mM; and the second wash buffer comprises at least about 750 mM NaCl. In some aspects, (i) the wash buffer comprises MgCl₂, wherein the concentration of Mg²⁺ in the wash buffer is at least about 350 mM; and the second wash buffer comprises at least about 750 mM NaCl. In some aspects, (i) the wash buffer comprises MgCl₂, wherein the concentration of Mg²⁺ in the wash buffer is at least about 375 mM; and the second wash buffer comprises at least about 750 mM NaCl. In some aspects, (i) the wash buffer comprises MgCl₂, wherein the concentration of Mg²⁺ in the wash buffer is at least about 400 mM; and the second wash buffer comprises at least about 750 mM NaCl.

In some aspects, (i) the wash buffer comprises MgCl₂, wherein the concentration of Mg²⁺ in the wash buffer is at least about 300 mM; and the second wash buffer comprises at least about 775 mM NaCl. In some aspects, (i) the wash buffer comprises MgCl₂, wherein the concentration of Mg²⁺ in the wash buffer is at least about 325 mM; and the second wash buffer comprises at least about 775 mM NaCl. In some aspects, (i) the wash buffer comprises MgCl₂, wherein the concentration of Mg²⁺ in the wash buffer is at least about 350 mM; and the second wash buffer comprises at least about 775 mM NaCl. In some aspects, (i) the wash buffer comprises MgCl₂, wherein the concentration of Mg²⁺ in the wash buffer is at least about 375 mM; and the second wash buffer comprises at least about 775 mM NaCl. In some aspects, (i) the wash buffer comprises MgCl₂, wherein the concentration of Mg²⁺ in the wash buffer is at least about 400 mM; and the second wash buffer comprises at least about 775 mM NaCl.

In some aspects, (i) the wash buffer comprises MgCl₂, wherein the concentration of Mg²⁺ in the wash buffer is at least about 300 mM; and the second wash buffer comprises at least about 800 mM NaCl. In some aspects, (i) the wash buffer comprises MgCl₂, wherein the concentration of Mg²⁺ in the wash buffer is at least about 325 mM; and the second wash buffer comprises at least about 800 mM NaCl. In some aspects, (i) the wash buffer comprises MgCl₂, wherein the concentration of Mg²⁺ in the wash buffer is at least about 350 mM; and the second wash buffer comprises at least about 800 mM NaCl. In some aspects, (i) the wash buffer comprises MgCl₂, wherein the concentration of Mg²⁺ in the wash buffer is at least about 375 mM; and the second wash buffer comprises at least about 800 mM NaCl. In some aspects, (i) the wash buffer comprises MgCl₂, wherein the concentration of Mg²⁺ in the wash buffer is at least about 400 mM; and the second wash buffer comprises at least about 800 mM NaCl.

In some aspects, (i) the wash buffer comprises MgCl₂, wherein the concentration of Mg²⁺ in the wash buffer is at least about 300 mM; and the second wash buffer comprises at least about 825 mM NaCl. In some aspects, (i) the wash buffer comprises MgCl₂, wherein the concentration of Mg²⁺ in the wash buffer is at least about 325 mM; and the second wash buffer comprises at least about 825 mM NaCl. In some aspects, (i) the wash buffer comprises MgCl₂, wherein the concentration of Mg²⁺ in the wash buffer is at least about 350 mM; and the second wash buffer comprises at least about 825 mM NaCl. In some aspects, (i) the wash buffer comprises MgCl₂, wherein the concentration of Mg²⁺ in the wash buffer is at least about 375 mM; and the second wash buffer comprises at least about 825 mM NaCl. In some aspects, (i) the wash buffer comprises MgCl₂, wherein the concentration of Mg²⁺ in the wash buffer is at least about 400 mM; and the second wash buffer comprises at least about 825 mM NaCl.

In some aspects, (i) the wash buffer comprises MgCl₂, wherein the concentration of Mg²⁺ in the wash buffer is at least about 300 mM; and the second wash buffer comprises at least about 850 mM NaCl. In some aspects, (i) the wash buffer comprises MgCl₂, wherein the concentration of Mg²⁺ in the wash buffer is at least about 325 mM; and the second wash buffer comprises at least about 850 mM NaCl. In some aspects, (i) the wash buffer comprises MgCl₂, wherein the concentration of Mg²⁺ in the wash buffer is at least about 350 mM; and the second wash buffer comprises at least about 850 mM NaCl. In some aspects, (i) the wash buffer comprises MgCl₂, wherein the concentration of Mg²⁺ in the wash buffer is at least about 375 mM; and the second wash buffer comprises at least about 850 mM NaCl. In some aspects, (i) the wash buffer comprises MgCl₂, wherein the concentration of Mg²⁺ in the wash buffer is at least about 400 mM; and the second wash buffer comprises at least about 850 mM NaCl.

In some aspects, (i) the wash buffer comprises MgCl₂, wherein the concentration of Mg²⁺ in the wash buffer is at least about 300 mM; and the second wash buffer comprises at least about 875 mM NaCl. In some aspects, (i) the wash buffer comprises MgCl₂, wherein the concentration of Mg²⁺ in the wash buffer is at least about 325 mM; and the second wash buffer comprises at least about 875 mM NaCl. In some aspects, (i) the wash buffer comprises MgCl₂, wherein the concentration of Mg²⁺ in the wash buffer is at least about 350 mM; and the second wash buffer comprises at least about 875 mM NaCl. In some aspects, (i) the wash buffer comprises MgCl₂, wherein the concentration of Mg²⁺ in the wash buffer is at least about 375 mM; and the second wash buffer comprises at least about 875 mM NaCl. In some aspects, (i) the wash buffer comprises MgCl₂, wherein the concentration of Mg²⁺ in the wash buffer is at least about 400 mM; and the second wash buffer comprises at least about 875 mM NaCl.

In some aspects, (i) the wash buffer comprises MgCl₂, wherein the concentration of Mg²⁺ in the wash buffer is at least about 300 mM; and the second wash buffer comprises at least about 900 mM NaCl. In some aspects, (i) the wash buffer comprises MgCl₂, wherein the concentration of Mg²⁺ in the wash buffer is at least about 325 mM; and the second wash buffer comprises at least about 900 mM NaCl. In some aspects, (i) the wash buffer comprises MgCl₂, wherein the concentration of Mg²⁺ in the wash buffer is at least about 350 mM; and the second wash buffer comprises at least about 900 mM NaCl. In some aspects, (i) the wash buffer comprises MgCl₂, wherein the concentration of Mg²⁺ in the wash buffer is at least about 375 mM; and the second wash buffer comprises at least about 900 mM NaCl. In some aspects, (i) the wash buffer comprises MgCl₂, wherein the concentration of Mg²⁺ in the wash buffer is at least about 400 mM; and the second wash buffer comprises at least about 900 mM NaCl.

In some aspects, the wash buffer comprises at least about 600 mM to about 1 M NaCl; and the second wash buffer comprises MgCl₂, wherein the concentration of Mg²⁺ in the wash buffer is at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, or at least about 500 mM Mg²⁺.

In some aspects, the wash buffer comprises at least about 600 mM to about 1 M NaCl; and the second wash buffer comprises MgCl₂, wherein the concentration of Mg²⁺ in the wash buffer is at least about 300 mM. In some aspects, the wash buffer comprises at least about 600 mM to about 1 M NaCl; and the second wash buffer comprises MgCl₂, wherein the concentration of Mg²⁺ in the wash buffer is at least about 325 mM. In some aspects, the wash buffer comprises at least about 600 mM to about 1 M NaCl; and the second wash buffer comprises MgCl₂, wherein the concentration of Mg²⁺ in the wash buffer is at least about 350 mM. In some aspects, the wash buffer comprises at least about 600 mM to about 1 M NaCl; and the second wash buffer comprises MgCl₂, wherein the concentration of Mg²⁺ in the wash buffer is at least about 375 mM. In some aspects, the wash buffer comprises at least about 600 mM to about 1 M NaCl; and the second wash buffer comprises MgCl₂, wherein the concentration of Mg²⁺ in the wash buffer is at least about 400 mM.

In some aspects, the wash buffer comprises at least about 750 mM NaCl; and the second wash buffer comprises MgCl₂, wherein the concentration of Mg²⁺ in the wash buffer is at least about 300 mM. In some aspects, the wash buffer comprises at least about 750 mM NaCl; and the second wash buffer comprises MgCl₂, wherein the concentration of Mg²⁺ in the wash buffer is at least about 325 mM. In some aspects, the wash buffer comprises at least about 750 mM NaCl; and the second wash buffer comprises MgCl₂, wherein the concentration of Mg²⁺ in the wash buffer is at least about 350 mM. In some aspects, the wash buffer comprises at least about 750 mM NaCl; and the second wash buffer comprises MgCl₂, wherein the concentration of Mg²⁺ in the wash buffer is at least about 375 mM. In some aspects, the wash buffer comprises at least about 750 mM NaCl; and the second wash buffer comprises MgCl₂, wherein the concentration of Mg²⁺ in the wash buffer is at least about 400 mM.

In some aspects, the wash buffer comprises at least about 800 mM NaCl; and the second wash buffer comprises MgCl₂, wherein the concentration of Mg²⁺ in the wash buffer is at least about 300 mM. In some aspects, the wash buffer comprises at least about 800 mM NaCl; and the second wash buffer comprises MgCl₂, wherein the concentration of Mg²⁺ in the wash buffer is at least about 325 mM. In some aspects, the wash buffer comprises at least about 800 mM NaCl; and the second wash buffer comprises MgCl₂, wherein the concentration of Mg²⁺ in the wash buffer is at least about 350 mM. In some aspects, the wash buffer comprises at least about 800 mM NaCl; and the second wash buffer comprises MgCl₂, wherein the concentration of Mg²⁺ in the wash buffer is at least about 375 mM. In some aspects, the wash buffer comprises at least about 800 mM NaCl; and the second wash buffer comprises MgCl₂, wherein the concentration of Mg²⁺ in the wash buffer is at least about 400 mM.

In some aspects, the wash buffer comprises at least about 850 mM NaCl; and the second wash buffer comprises MgCl₂, wherein the concentration of Mg²⁺ in the wash buffer is at least about 300 mM. In some aspects, the wash buffer comprises at least about 850 mM NaCl; and the second wash buffer comprises MgCl₂, wherein the concentration of Mg²⁺ in the wash buffer is at least about 325 mM. In some aspects, the wash buffer comprises at least about 850 mM NaCl; and the second wash buffer comprises MgCl₂, wherein the concentration of Mg²⁺ in the wash buffer is at least about 350 mM. In some aspects, the wash buffer comprises at least about 850 mM NaCl; and the second wash buffer comprises MgCl₂, wherein the concentration of Mg²⁺ in the wash buffer is at least about 375 mM. In some aspects, the wash buffer comprises at least about 850 mM NaCl; and the second wash buffer comprises MgCl₂, wherein the concentration of Mg²⁺ in the wash buffer is at least about 400 mM.

In some aspects, the NaCl of the second wash buffer, as described herein, can be replaced by NaBr, KCl, or KBr. As such, in some aspects, the second wash buffer comprises NaBr. In some aspects, the second wash buffer comprises KCl. In some aspects, the second wash buffer comprises KBr. In some aspects, the second wash buffer comprises at least about 600 mM to about 1 M NaBr, KCl, or KBr. In some aspects, the second wash buffer comprises NaBr, KCl, or KBr at a concentration of at least about 600 mM, at least about 625 mM, at least about 650 mM, at least about 675 mM, at least about 700 mM, at least about 725 mM, at least about 750 mM, at least about 775 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1 M NaBr, KCl, or KBr. In some aspects, the second wash buffer comprises at least about 600 mM NaBr, KCl, or KBr. In some aspects, the second wash buffer comprises at least about 650 mM NaBr, KCl, or KBr. In some aspects, the second wash buffer comprises at least about 700 mM NaBr, KCl, or KBr. In some aspects, the second wash buffer comprises at least about 750 mM NaBr, KCl, or KBr. In some aspects, the second wash buffer comprises at least about 775 mM NaBr, KCl, or KBr. In some aspects, the second wash buffer comprises at least about 800 mM NaBr, KCl, or KBr. In some aspects, the second wash buffer comprises at least about 825 mM NaBr, KCl, or KBr. In some aspects, the second wash buffer comprises at least about 850 mM NaBr, KCl, or KBr. In some aspects, the second wash buffer comprises at least about 875 mM NaBr, KCl, or KBr. In some aspects, the second wash buffer comprises at least about 900 mM NaBr, KCl, or KBr. In some aspects, the second wash buffer comprises at least about 950 mM NaBr, KCl, or KBr. In some aspects, the second wash buffer comprises at least about 1 M NaBr, KCl, or KBr.

Some aspects of the present disclosure are directed to methods comprising contacting a chromatography resin associated with the sample with a wash buffer; wherein the wash buffer comprises MgCl₂, CaCl₂, NaCl, and/or KCl and does not comprise a nuclease. In some aspects, the wash buffer comprises Mg²⁺, e.g., MgCl₂ of MgBr₂. In some aspects, the wash buffer comprises Ca²⁺, e.g., CaCl₂ or CaBr₂. In some aspects, a salt comprising a cation other than Mg²⁺ or Ca²⁺ can be used in the wash. In some aspects, the cation can comprise a monovalent cation. In some aspects, the monovalent cation is selected from Li⁺, K⁺, Na⁺, NH4⁺, Cu⁺, and any combination thereof. In some aspects, the wash buffer comprises Na⁺, e.g., NaCl or NaBr. In some aspects, the wash buffer comprises Na⁺, e.g., KCl or KBr. In some aspects, the cation can comprises a divalent cation. In some aspects, the divalent cation is selected from Co²⁺, Ni²⁺, Zn²⁺, Ba²⁺, Sr²⁺, Al²⁺, Ag²⁺, Cu²⁺, Mn²⁺, and any combination thereof.

### II.B. Chromatography Resins

Certain aspects of the present disclosure are directed to methods of preparing purified EVs, e.g., exosomes, from a sample comprising EVs, e.g., exosomes, and one or more nucleic acid molecules, comprising: (i) contacting the sample with a chromatography resin and (ii) contacting the chromatography resin with a wash buffer, e.g., a wash buffer comprising MgCL₂, CaCl₂, KCl, and/or NaCl, wherein the (ii) washing follows the (i) contacting. Other aspects of the present disclosure are directed to methods of preparing purified EVs from a sample comprising EVs and one or more nucleic acid molecules, comprising: (i) contacting the sample with a chromatography resin, (ii) eluting an eluent form the chromatography resin, wherein the eluent comprises the EVs, e.g., exosomes, and (iii) contacting the eluent with a wash buffer, e.g., a wash buffer comprising MgCL₂, CaCl₂, KCl, and/or NaCl. These methods reduce the level of contaminant nucleic acid molecules in a sample comprising EVs, e.g., exosomes, and one or more nucleic acid molecules. As such, the methods disclosed herein can be performed using any chromatography resin.

In some aspects, the chromatography resin comprises an ion exchange chromatography resin. In certain aspects, the chromatography resin comprises an anion exchange (AEX) resin. In some aspects, the chromatography resin comprises a cation exchange (CEX) resin. In some aspects, the chromatography resin comprises a hydrophobic interaction resin. In some aspects, the chromatography resin comprises a hydrophobic charge induction chromatography resin. In some aspects, the chromatography resin comprises a mixed mode resin. In some aspects, the chromatography resin comprises an immobilized metal affinity resin. In some aspects, the chromatography resin comprise a ceramic hydroxyapatite resin. In some aspects, the chromatography resin comprise a fluoro hydroxyapatite resin. In some aspects, the chromatography resin comprise a ceramic fluoroapatite. In some aspects, the chromatography resin comprise a combination of one or more chromatography resin disclosed herein. In some aspects, the chromatography resin comprises a mixed-mode chromatography (MMC) resin.

In certain aspects, the chromatography resin comprises an ion exchange chromatography resin (e.g. an AEX or a CEX chromatography resin). In certain aspects, the method comprises (i) contacting the sample with an AEX chromatography resin and (ii) contacting the chromatography resin with a wash buffer, e.g., a wash buffer comprising MgCL₂, CaCl₂, KCl, and/or NaCl. In some aspects, the method further comprises subjecting the sample to one or more additional chromatography resins. In some aspects, the one or more additional chromatography resins comprises an additional AEX resin, a CEX resin, an MMC resin, a hydrophobic charge induction chromatography resin, a hydrophobic interaction chromatography resin, an immobilized metal affinity resin, or any combination thereof. In some aspects, the sample is contacted with a CEX resin after the AEX resin. In some aspects, wherein the sample is contacted with an MMC resin after the CEX resin. In some aspects, the sample is contacted with an MMC resin after the AEX resin. In certain aspects, the sample is contacted with (i) the AEX resin, (ii) a CEX resin, and (iii) an MMC resin, in the sequence (i), (ii), then (iii).

In some aspects, the sample is contacted with (a) an AEX resin, (b) a CEX resin, and (c) an MMC resin; wherein after the sample is contacted with the AEX resin, the AEX resin is contacted with a wash buffer, e.g., a wash buffer comprising MgCL₂, CaCl₂, KCl, and/or NaCl. In some aspects, the sample is contacted with (a) an AEX resin, (b) a CEX resin, and (c) an MMC resin; wherein after the sample is contacted with the CEX resin, the CEX resin is contacted with a wash buffer, e.g., a wash buffer comprising MgCL₂, CaCl₂, KCl, and/or NaCl. In some aspects, the sample is contacted with (a) an AEX resin, (b) a CEX resin, and (c) an MMC resin; wherein after the sample is contacted with the MMC resin, the MMC resin is contacted with a wash buffer, e.g., a wash buffer comprising MgCL₂, CaCl₂, KCl, and/or NaCl.

In some aspects, the sample is contacted with (a) an AEX resin, (b) a CEX resin, and (c) an MMC resin; wherein (i) after the sample is contacted with the AEX resin, the AEX resin is contacted with a wash buffer, e.g., a wash buffer comprising MgCL₂, CaCl₂, KCl, and/or NaCl; and wherein (ii) after the sample is contacted with the CEX resin, the CEX resin is contacted with a wash buffer, e.g., a wash buffer comprising MgCL₂, CaCl₂, KCl, and/or NaCl. In some aspects, the sample is contacted with (a) an AEX resin, (b) a CEX resin, and (c) an MMC resin; wherein (i) after the sample is contacted with the AEX resin, the AEX resin is contacted with a wash buffer, e.g., a wash buffer comprising MgCL₂, CaCl₂, KCl, and/or NaCl; and wherein (ii) after the sample is contacted with the MMC resin, the MMC resin is contacted with a wash buffer, e.g., a wash buffer comprising MgCL₂, CaCl₂, KCl, and/or NaCl. In some aspects, the sample is contacted with (a) an AEX resin, (b) a CEX resin, and (c) an MMC resin; wherein (i) after the sample is contacted with the CEX resin, the CEX resin is contacted with a wash buffer, e.g., a wash buffer comprising MgCL₂, CaCl₂, KCl, and/or NaCl; and wherein (ii) after the sample is contacted with the MMC resin, the MMC resin is contacted with a wash buffer, e.g., a wash buffer comprising MgCL₂, CaCl₂, KCl, and/or NaCl. In some aspects, the sample is contacted with (a) an AEX resin, (b) a CEX resin, and (c) an MMC resin; wherein (i) after the sample is contacted with the AEX resin, the AEX resin is contacted with a wash buffer, e.g., a wash buffer comprising MgCL₂, CaCl₂, KCl, and/or NaCl; wherein (ii) after the sample is contacted with the CEX resin, the CEX resin is contacted with a wash buffer, e.g., a wash buffer comprising MgCL₂, CaCl₂, KCl, and/or NaCl; and wherein (iii) after the sample is contacted with the MMC resin, the MMC resin is contacted with a wash buffer, e.g., a wash buffer comprising MgCL₂, CaCl₂, KCl, and/or NaCl.

In some aspects, the method comprises (i) CEX-AEX-MMC; (ii) CEX-MMC-AEX; (iii) AEX-CEX-MMC; (iv) AEX-MMC-CEX; (v) MMC-CEX-AEX; or (vi) MMC-AEX-CEX. In other aspects, the method comprises CEX-AEX-MMC. In other aspects, the method comprises AEX-CEX-MMC.

For each chromatography (e.g., CEX, AEX, and MMC), various buffers (loading buffer, elution buffer, wash buffer, etc.) and conditions can be used to maximize the yield while removing the impurities as much as possible. In some aspects, each of the chromatography comprises a loading buffer, an elution buffer, and/or a wash buffer. In some aspects, the loading buffer and the elution buffer can be the same. In other aspects, the elution buffer and the wash buffer can be the same. In other aspects, the loading and wash buffers can be the same. In some aspects, the loading and wash buffers can be the same, but the elution buffer is different from the loading and wash buffers. In other aspects, the loading buffer, the elution buffer, and the wash buffer are the same.

In some aspects, CEX elution conditions can be designed to be the same as the AEX load conditions enabling straight through operation. In some aspects, CEX elution conditions can be designed to be the same as the AEX load conditions enabling straight through operation while the CEX loading conditions (e.g., a lower pH than the elution buffer) are different from the CEX elution conditions. In some aspects, AEX elution conditions can be designed to be the same as the MMC load conditions enabling straight through operation. Straight through processing can also be accomplished by integrated dilution or in-line titration of an elution and/or a load. In some aspects, CEX and AEX columns can be duplexed (placed inline in series) to enable operation of both columns in a single unit operation; the CEX column operated in flow-through or weak partitioning mode with the flow-through directly binding to the downstream AEX column. In some aspects, the product can be eluted from the AEX with a separate elution. In some aspects, to prevent fouling and maximize reuse of the downstream column, the two columns can be separated for strips and/or other phases.

In some aspects, selective loading, capture, elution, and/or wash can be achieved by changing salt, phosphate, or calcium concentrations, changing pH, altering temperature, adding organic modifiers, organic solvents, small molecules, detergents, zwitterions, amino acids, polymers, polyols (sucrose, glucose, trehalose, mannose, sorbitol, mannitol, glycerol, etc.), anti-oxidants (e.g., methionine), EDTA, EGTA, Polysorbate 20, Polysorbate 80, ethylene glycol, propylene glycol, polyethylene glycol, polypropylene glycol, and/or urea, adding excipients that alter the surface tension of the solution, adding excipients that alter the polarity of the solution, altering the residence time to take advantage of differential desorption rates between impurities and EVs, adding excipients that modulate the structure of the EVs, or any combination of the above.

In some aspects, loading, capture, elution, and/or wash can be achieved by using EDTA to inhibit any potential contaminating metalloproteases. In some aspects, the EDTA is present at a concentration of from about 0.0001M to about 1M in a buffer, e.g., an elution buffer for the AEX. In some aspects, the EDTA is present at a concentration of from about 0.001M to about 1M. In some aspects, the EDTA is present at a concentration of from about 0.001M to about 0.1M, from about 0.001M to about 0.09M, from about 0.002M to about 0.08M, from about 0.003M to about 0.07M, from about 0.004M to about 0.06M, from about 0.005M to about 0.05M, from about 0.006M to about 0.04M, from about 0.007M to about 0.03M, from about 0.008M to about 0.02M, or from about 0.009M to about 0.01M. In some aspects, the EDTA is present at a concentration of from about 0.01M to about 0.1M. In some aspects, the EDTA is present at a concentration of about 0.001M. In some aspects, the EDTA is present at a concentration of about 0.001M. In some aspects, the EDTA is present at a concentration of about 0.005M. In some aspects, the EDTA is present at a concentration of about 0.01M. In some aspects, the EDTA is present at a concentration of about 0.02M. In some aspects, the EDTA is present at a concentration of about 0.003M. In some aspects, the EDTA is present at a concentration of about 0.004M. In some aspects, the EDTA is present at a concentration of about 0.05M. In some aspects, the EDTA is present at a concentration of about 0.06M. In some aspects, the EDTA is present at a concentration of about 0.07M. In some aspects, the EDTA is present at a concentration of about 0.08M In some aspects, the EDTA is present at a concentration of about 0.09M In some aspects, the EDTA is present at a concentration of about 0.1M.

In some aspects, selective loading, elution, and/or wash of EVs can be achieved by increasing the concentration of a monovalent salt (e.g., sodium chloride, potassium chloride, sodium bromide, lithium chloride, sodium iodide, potassium bromide, lithium bromide, sodium fluoride, potassium fluoride, lithium fluoride, lithium iodide, sodium acetate, potassium acetate, lithium acetate, and potassium iodide), a divalent or trivalent salt (e.g., calcium chloride, magnesium chloride, calcium sulfate, sodium sulfate, magnesium sulfate, chromium trichloride, chromium sulfate, sodium citrate, iron (III) chloride, yttrium (III) chloride, potassium phosphate, potassium sulfate, sodium phosphate, ferrous chloride, calcium citrate, magnesium phosphate, and ferric chloride), or a combination thereof, in the elution buffer for a chromatography (e.g., CEX, AEX, and/or MMC), through the use of an increasing gradient (step or linear) of a monovalent salt (e.g., sodium chloride, potassium chloride, sodium bromide, lithium chloride, sodium iodide, potassium bromide, lithium bromide, sodium fluoride, potassium fluoride, lithium fluoride, lithium iodide, sodium acetate, potassium acetate, lithium acetate, and potassium iodide), a divalent or trivalent salt (e.g., calcium chloride, magnesium chloride, calcium sulfate, sodium sulfate, magnesium sulfate, chromium trichloride, chromium sulfate, sodium citrate, iron (III) chloride, yttrium (III) chloride, potassium phosphate, potassium sulfate, sodium phosphate, ferrous chloride, calcium citrate, magnesium phosphate, and ferric chloride), or a combination thereof, at a fixed pH. In some aspects, one or more buffers, e.g., elution buffer or loading buffer, e.g., elution buffer for AEX, loading buffer for AEX, comprises NaCl.

In some aspects, substantial EV purity can be achieved by flowing through impurities during the column loading phase, eluting impurities during selective excipient washes, and/or by selectively eluting a target during elution while leaving additional impurities bound to the column. Absorbance measurements of column eluates can suggest changes (e.g., a significant reduction) in concentrations of proteins and nucleic acids. In some aspects, the interaction between the chromatographic resins (e.g., CEX, AEX, and/or MMC) and EVs is sufficient to enable direct capture from cell culture, clarified cell culture, concentrated cell culture, or partially purified in-process pools.

In some aspects, excipients can be used for the washing step for one or more chromatography processes (e.g., CEX, AEX, and/or MMC). Excipient washes can improve purity or further aid in enriching, depleting, or isolating sub-populations of EVs. In some aspects, the excipient can be a solution having specific pH ranges, salts, organic solvents, small molecules, detergents, zwitterions, amino acids, polymers, and any combination of the above.

In some aspects, the excipient can comprise arginine, lysine, glycine, histidine, calcium, sodium, lithium, potassium, iodide, magnesium, iron, zinc, manganese, urea, propylene glycol, aluminum, ammonium, guanidinium polyethylene glycol, EDTA, EGTA, a detergent, chloride, sulfate, carboxylic acids, sialic acids, phosphate, acetate, glycine, borate, formate, perchlorate, bromine, nitrate, dithiothreitol, beta mercaptoethanol, or tri-n-butyl phosphate.

In some aspects, the excipient can also comprise a detergent. In some aspects, the detergent is selected from cetyl trimethylammonium chloride, octoxynol-9, TRITON™ X-100 (i.e., polyethylene glycol p-(1,1,3,3-tetramethylbutyl)-phenyl ether) and TRITON™ CG-110 available from Sigma-Aldrich; sodium dodecyl sulfate; sodium lauryl sulfate; deoxycholic acid; Polysorbate 80 (i.e., Polyoxyethylene (20) sorbitan monooleate); Polysorbate 20 (i.e., Polyoxyethylene (20) sorbitan monolaurate); alcohol ethoxylate; alkyl polyethylene glycol ether; decyl glucoside; octoglucosides; SafeCare; ECOSURF™ EH9, ECOSURF™ EH6, ECOSURF™ EH3, ECOSURF™ SA7, and ECOSURF™ SA9 available from DOW Chemical; LUTENSOL™ M5, LUTENSOL™ XL, LUTENSOL™ XP and APG™ 325N available from BASF; TOMADOL™ 900 available from AIR PRODUCTS; NATSURF™ 265 available from CRODA; SAFECARE™1000 available from Bestchem, TERGITOL™ L64 available from DOW; caprylic acid; CHEMBETAINE™ LEC available from Lubrizol; Mackol DG, and mixtures thereof.

In some aspects of the multistep process, any unit operation (i.e., any step in the process) can be run in batch, semi-batch, semi-continuous, or continuous mode. In some aspects, surge tanks can be employed to enable semi-continuous or continuous processing.

In other aspects, the sequence of the chromatography process (e.g., CEX-AEX-MMC or AEX-CEX-MMC) can be repeated at least two times, at least three times, at least four times, at least five times, at least six times, at least seven times, at least eight times, at least nine times, at least ten times, at least 11 times, at least 12 times, at least 13 times, at least 14 times, at least 15 times, at least 16 times, at least 17 times, at least 18 times, at least 19 times, or at least 20 times.

In some aspects, AEX and MMC columns are duplexed (placed inline in series) to enable operation of both columns in a single until operation; the AEX column is operated in bind/elute mode with the elution loaded directly onto the MMC column operation in flow-through or weak partitioning mode. In some aspects, to prevent fouling and maximize reuse of the downstream column, the two columns can be separated for strips or other phases.

In some aspects, the methods of the present disclosure comprises two or more processes (e.g., chromatographies) connected for continuous manufacturing (e.g., purification). In some aspects, the continuous manufacturing (e.g., purification) processes are integrated with the bioreactor that produces the EVs.

### II.B.1. CEX Chromatography Resins

The CEX process is a form of ion exchange chromatography that separates samples based on their net surface charge. CEX specifically uses negatively charged ligands having affinity to targets having positive surface charges. Without being bound by a particular theory, EVs may be amphoteric and present positive surface charges that can be exploited for CEX purification under certain purification conditions. The method can rely on positive charges of the surface proteins on the EVs that contain basic amino acids such as lysine and arginine and/or are complexed with bivalent positively charged metals. In addition, the presence of chromatin can offer an array of basic histone proteins for CEX binding.

Various CEX resins can be used in the CEX process. In some aspects, CEX resins comprise a CEX ligand and a base matrix. In some aspects, the base matrix can comprise membranes, monoliths, hydrogels, nanofiber, composite resins, beaded resins, beaded resins with inert porous shells, an/or any other absorptive or convective media. In other aspects the base matrix can comprise materials such as cellulose, agarose, polystyrene derivatives, polyvinyl ether, silica, methacrylate derivatives, glass, ceramic hydroxyapatite, acrylamide, other backbones commonly used in chromatography and known by those of skill in the art, and/or mixtures thereof.

Various CEX ligands can be used in the CEX process. In some aspects, the CEX ligands comprise sulfate, sulfopropyl, sulfobutyl, sulfoisobutyl, sulfoethyl, sulfonate, sulfonic acid, carboxymethyl, carboxylic acid, glutamic acid, aspartic acid, histidine, hydroxyl, and/or phosphate ligands. In some aspects, CEX ligands are used together with other conventional chromatography ligands such as sulfate ligands, tertiary amine ligands, quaternary amine ligands, diethaminoethyl ligands, butyl ligands, hexyl ligands, ether ligands, polypropylene glycol ligands, phenyl ligands, ceramic hydroxy apatite ceramic fluoroapatite ligands, amino acid ligands, or any combination thereof. In some aspects, commercially available chromatography ligands are used, for example, those formulated as SP SEPHAROSE™ FF, SP SEPHAROSE™ HP, SP SEPHAROSE™ BB, SP SEPHAROSE™ XL, CM SEPHAROSE™ FF, CM SEPHAROSE™ HP, SOURCE™ 15S, SOURCE™ 30S, CAPTO™ S, MacroCap SP, CAPTO™ SP ImpRes, or CAPTO™ S ImpAct available from GE Healthcare; FRACTOGEL® EMD SO3− (M), FRACTOGEL® EMD SO3− (S), FRACTOGEL® EMD SE Hicap (M), ESHMUNO® S, or ESHMUNO® CPX available from Merck Millipore; TOYOPEARL® CM-650C, TOYOPEARL® CM-650M, TOYOPEARL® CM-650S, TOYOPEARL® SP-650C, TOYOPEARL® SP-650M, TOYOPEARL® SP-650S, TOYOPEARL® SP-550C, TOYOPEARL® MEGACAP® II SP-550 EC, TOYOPEARL® GIGACAP® S-650M, TOYOPEARL® GIGACAP® CM-650M, or TOYOPEARL® GIGACAP® S-650S available from Tosoh Bioscience; MACRO-PREP® High S, MACRO-PREP® 25 S, MACRO-PREP® CM, UNOSPHERE™ S, NUVIA™ S, or NUVIA™ HR-S available from BioRad Laboratories; S HYPERCEL™, CM Ceramic HYPERD® F, S Ceramic HYPERD® 20, S Ceramic HYPERD® F, CMM HYPERCEL™, or HYPERCEL™ STAR CEX, available from Pall Corporation; POROS® 50 HS, POROS® 20 HS, or POROS® XS, available from Thermo Fisher Scientific/Life Technologies; PL-SCX 1000 Å30 μm or PL-SCX 1000 Å 10 μm, available from Agilent Technologies; CELLUFINE® MAX S-r, CELLUFINE® MAX S-h, or CELLUFINE® C-500 (m), available from JNC Corporation; BAKERBOND™ POLYABx or BAKERBOND™ POLYABx, available from Avantor Pharmaceutical Materials; YMC—BioPro S30, YMC—BioPro S75, YMC—BioPro SmartSep S10, YMC—BioPro SmartSep S30, or YMC—BioPro SmartSep S30, available from YMC; or PRAESTO™ SP45, PRAESTO™ SP65, or PRAESTO™ SP65, available from Purolite. In some aspects, the CEX resin used in the purification process can be POROS® XS, available from Thermo Fisher Scientific/Life Technologies. In some aspects, a CEX ligand for the CEX process is POROS® XS. In some aspects, a CEX ligand for the CEX process is CMM HyperCel™.

Interactions between the ligands and EVs are influenced by several factors, such as cation exchangers, flow rate, particle size of the resin, binding capacity, or any combination thereof. In certain aspects the present disclosure further provides conditions where EVs can be effectively isolated, purified or sub-fractionated with cation exchange ligands. In some aspects, the binding of EVs to CEX ligands is strengthened in lower pH. In some aspects, the pH of the CEX loading buffer is from about 5.0 to about 7.0.

In some aspects, the binding of EVs to CEX ligands is strengthened in lower salt concentrations. In some aspects, the CEX loading buffer comprises a salt concentration from about 10 mM to about 300 mM, from about 20 mM to about 300 mM, from about 30 mM to about 250 mM, from about 40 mM to about 200 mM, from about 50 mM to about 150 mM, from about 60 mM to about 150 mM, from about 70 mM to about 150 mM, from about 80 mM to about 150 mM, from about 90 mM to about 150 mM, from about 100 mM to about 150 mM, from about 110 mM to about 150 mM, or from about 120 mM to about 150 mM. In other aspects, the CEX loading buffer comprises a salt concentration of about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, or about 200 mM. In some aspects, the salt concentration of the CEX loading buffer is about 130 mM, about 135 mM, about 137 mM, or about 140 mM.

In some aspects, CEX is performed in a bind-elute mode. In some aspects, CEX is performed in a flow-through mode. In some aspects, CEX is performed in a weak-partitioning mode, where the EVs are bound more weakly that impurities which bind more strongly to the CEX resin.

In the weak-partitioning mode, at least some desired EVs and at least some undesired EVs or impurities, both bind to the chromatographic medium. However, undesired EVs or impurities bind more tightly to the medium. Unbound, desired EVs pass through the medium and are recovered from the column effluent. The binding between EVs and the chromatographic medium is intermediate in comparison to bind-elute and flow-through modes.

In some aspects, a loading phase can be followed by a wash phase to increase recovery of the desired product. Washing can be done with a washing buffer identical to or different from the loading buffer. When different, the wash buffer is different from the loading buffer in terms of composition or pH.

In some aspects, the pH of the CEX wash buffer is higher than the pH of the CEX loading buffer.

In some aspects, the CEX wash buffer comprises a salt concentration from about 300 mM to about 5M, from about 300 mM to about 4M, from about 300 mM to about 3M, from about 400 mM to about 3M, from about 500 mM to about 3M, from about 600 mM to about 2.5M, from about 700 mM to about 2.5M, from about 800 mM to about 2.5M, from about 900 mM to about 2.5M, from about 1M to about 2.4M, from about 1M to about 2.3M, or from about 1.5M to about 2M. In other aspects, the CEX wash buffer comprises a salt concentration of about 300 mM, about 400 mM, about 500 mM, about 600 mM, about 700 mM, about 800 mM, about 900 mM, about 1M, about 1.1M, about 1.2M, about 1.3M, about 1.4M, about 1.5M, about 1.6M, about 1.7M, about 1.8M, about 1.9M, about 2.0M, about 2.1M, about 2.2M, about 2.3M, about 2.4M, about 2.5M, about 2.6M, about 2.7M, about 2.8M, about 2.9M, or about 3.0M. In some aspects, the salt concentration of the CEX wash buffer is about 1M, about 1.5M, about 2.0M, or about 2.5M. In some aspects, the salt concentration of the CEX wash buffer is about 2M.

In certain aspects, various weak-partitioning purification methods, well-known in the art, can be combined with the methods disclosed in this application. For example, in some aspects, methods for identifying ideal conditions for the weak-partitioning mode or purification methods disclosed in the U.S. Publication No. 2007/0060741, which is incorporated by reference in its entirety herein, can be used.

In certain aspects the CEX process is repeated multiple times. In some aspects, the CEX process is repeated at least two times, at least three times, at least four times, at least five times, at least six times, at least seven times, at least eight times, at least nine times, at least ten times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, at least 31 times, at least 32 times, at least 33 times, at least 34 times, at least 35 times, at least 36 times, at least 37 times, at least 38 times, at least 39 times, at least 40 times, at least 41 times, at least 42 times, at least 43 times, at least 44 times, at least 45 times, at least 46 times, at least 47 times, at least 48 times, at least 49 times, at least 50 times. In some aspects, the CEX process is repeated at least three times. In some aspects, the CEX process is repeated at least four times. In some aspects, the CEX process is repeated at least five times. In some aspects, the CEX process is repeated at least six times.

### II.B.2. Anion Exchange Chromatography (AEX) Chromatography Resins

In certain aspects of the present disclosure, the chromatography resin comprises an AEX chromatography resin. AEX is another form of ion exchange chromatography that separates samples based on their surface charge. AEX uses positively charged ligands having affinity to targets having negative surface charges. In some aspects, the AEX can be performed on the sample comprising EVs after the sample has been subjected to a CEX. In other aspects, the AEX can be performed on the sample comprising EVs before the sample has been subjected to a CEX. In some aspects, the AEX can be performed on the sample comprising EVs before the sample has been subjected to an MMC. In some aspects, the AEX can be performed on the sample comprising EVs after the sample has been subjected to an MMC.

In some aspects, AEX is performed in a weak-partitioning mode. In some aspects, AEX is performed in flow-through mode. In some aspects, AEX is performed in a bind-elute mode.

In bind-elute mode, desired EVs bind to chromatographic medium and are eluted from the medium by elution buffers. These methods generally comprise the steps of applying or loading a sample comprising EVs, optionally washing away unbound sample components using appropriate buffers that maintain the binding interaction between EVs and affinity ligands and eluting (dissociating and recovering) EVs from the immobilized ligands by altering buffer conditions so that the binding interaction no longer occurs.

In some aspects, exchange resin can be eluted with a particular elution buffer and selected fractions of the eluate can be concentrated (e.g., by dialysis) to provide an enriched EV preparation. In certain aspects, the AEX resin used in the scalable method is of a sufficient size to accommodate large scale volumes of conditioned culture media. In other aspects, a second elution of the collected fractions from a first passage over an anion exchange column can be performed. In some aspects, the AEX is repeated at least two times, at least three times, at least four times, at least five times, at least six times, at least seven times, at least eight times, at least nine times, at least ten times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, at least 31 times, at least 32 times, at least 33 times, at least 34 times, at least 35 times, at least 36 times, at least 37 times, at least 38 times, at least 39 times, at least 40 times, at least 41 times, at least 42 times, at least 43 times, at least 44 times, at least 45 times, at least 46 times, at least 47 times, at least 48 times, at least 49 times, at least 50 times. In some aspects, the AEX is repeated at least three times. In some aspects, the AEX is repeated at least four times. In some aspects, the AEX is repeated at least five times. In some aspects, the AEX is repeated at least six times.

AEX resin refers to a solid phase which is positively charged, e.g. having one or more positively charged ligands. In some aspects, the ligands are selected from diethylaminopropyl, diethylaminoethyl, quaternary aminoethyl, quaternary ammonium, carboxymethyl, carboxylic acid, glutamic acid, aspartic acid, histidine, hydroxyl, phosphate, tertiary amines, quaternary amines, diethaminoethyl, dimethylaminoethyl, trimethylaminoethyl, an amino acid ligand, or combinations thereof. Commercially available anion exchange resins include DEAE cellulose, QAE SEPHADEX and FAST Q SEPHAROSE (Pharmacia). In certain aspects the chromatography ligands can be bound to a base matrix. In some aspects, the base matrix can comprise monoliths, hydrogels, porous devices, nanofibers, composite resins, beaded resins, beaded resin with inert porous shells, and/or any other solid or porous support. In some aspects, the base matrix can comprise cellulose, agarose, polystyrene derivatives, polyvinyl ether, silica, methacrylate derivatives, glass, ceramic hydroxyapatite, ceramic fluoroapatite, acrylamide, and/or other backbones commonly used in chromatography.

Examples of anion exchange resins include, but are not limited to: Q SEPHAROSE™ FF, Q SEPHAROSE™ HP, Q SEPHAROSE™ BB, Q SEPHAROSE™ XL, DEAE SEPHAROSE™ FF, ANX SEPHAROSE™ 4FF low sub, ANX SEPHAROSE™ 4FF high sub, SOURCE™ 15Q, SOURCE™ 30Q, CAPTO™ Q, CAPTO™ DEAE, or CAPTO™ Q ImpRes, available from GE Healthcare; FRACTOGEL® EMD DEAE (M), FRACTOGEL® EMD TMAE (M), FRACTOGEL® EMID TMAE (S), FRACTOGEL® EMD TMAE Hicap (M), FRACTOGEL® EMD TMAE Medcap (M), ESHMUNO® Q or ESHMUNO® Q, available from Merck Millipore; TOYOPEARL® DEAE-650C, TOYOPEARL® DEAE-650M, TOYOPEARL® DEAE-650S, TOYOPEARL® SuperQ-650C, TOYOPEARL® SuperQ-650M, TOYOPEARL® SuperQ-650S, TOYOPEARL® QAE-550C, TOYOPEARL® GIGACAP® Q-650M, TOYOPEARL® Q-600C AR, TOYOPEARL® GIGACAP® DEAE-650M, TOYOPEARL® GIGACAP® Q-650S, TOYOPEARL® NH2-750F, TSKGEL® SuperQ-5PW (20 μm), or TSKGEL® SuperQ-5PW (30 μm), available from Tosoh Bioscience; MACRO-PREP® DEAE, MACRO-PREP® High Q, MACRO-PREP® 25 Q, UNOSPHERE™ Q or NUVIA™ Q, available from BioRad Laboratories; Q HYPERCEL™, DEAE Ceramic HYPERD® F, Q Ceramic HYPERD® 20, Q Ceramic HYPERD® F, or HYPERCEL™ STAR AX, available from Pall Corporation; POROS® 50 HQ, POROS® 50 PI, POROS® 50 D, POROS® 20 HQ, or POROS® XQ, available from Thermo Fisher Scientific/Life Technologies; DEAE PuraBead HF, available from Prometic Bioseparations; PL-SAX 1000 Å 30 μm, or PL-SAX 1000 Å 10 μm, available from Agilent Technologies; CELLUFINE® MAX Q-h, or CELLUFINE® Q-500 (m), available from INC Corporation; BAKERBOND™ POLYQUAT, BAKERBOND™ POLYPEI, or BAKERBOND™ POLYPEI, available from Avantor Pharmaceutical Materials; YMC—BioPro Q30, YMC—BioPro Q75, YMC—BioPro SmartSep Q10, or YMC—BioPro SmartSep Q30, available from YMC; Sartobind Q, available from 8 mm; or PRAESTO™ Q65 or PRAESTO™ Q90, available from Purolite. In some aspects the AEX resin can be Sartobind Q, available from 8 mm. In some aspects, an AEX resin for the AEX process is SARTOBIND® Q (8 mm).

In some aspects, binding of EVs to AEX ligands is strengthened in higher pH compared to the CEX process as described herein. In other aspects, binding of EVs to AEX ligands is strengthened in lower salt conditions compared to one or more chromatography processes, (e.g., CEX and/or MMC). Accordingly, the methods can further comprise the step of changing (raising or lowering) the salt concentration or pH of the sample before loading the sample to the AEX resin. In some aspects, the pH and the salt concentration for the AEX process are selected for inducing precipitation of contaminant proteins. In some aspects, the AEX chromatography is conducted at a pH from about 7 to about 10. In some aspects, the pH of the AEX loading buffer is about 7.4.

In some aspects, the AEX loading buffer comprises a salt concentration from about 10 mM to about 1000 mM, from about 50 mM to about 900 mM, from about 60 mM to about 800 mM, from about 70 mM to about 700 mM, from about 80 mM to about 700 mM, from about 80 mM to about 800 mM, from about 90 mM to about 700 mM, from about 100 mM to about 700 mM, from about 150 mM to about 700 mM, from about 200 mM to about 700 mM, from about 300 mM to about 600 mM, from about 400 mM to about 600 mM, from about 500 mM to about 600 mM, from about 500 mM to about 700 mM, or from about 500 mM to about 800 mM. In some aspects, the AEX loading buffer comprises a salt concentration of about 100 mM, about 150 mM, about 200 mM, about 250 mM, about 300 mM, about 350 mM, about 400 mM, about 450 mM, about 500 mM, about 550 mM, about 600 mM, about 650 mM, about 700 mM, about 800 mM, about 900 mM, or about 1M.

In some aspects the AEX elution buffer comprises a salt concentration from about 600 mM to about 1500 mM, from about 700 mM to about 1400 mM, from about 800 mM to about 1300 mM, from about 900 mM to about 1200 mM, from about 800 mM to about 1500 mM, from about 700 mM to about 1500 mM, from about 800 mM to about 1400 mM, from about 600 mM to about 1300 mM, from about 600 mM to about 1400 mM, from about 600 mM to about 1200 mM, from about 600 mM to about 1100 mM, or from about 1000 mM to about 1500 mM.

In other aspects, the AEX wash buffer comprises a salt concentration from about 1M to about 3M, from about 1M to about 2.9M, from about 1.1M to about 2.9M, from about 1.5M to about 2.5M, from about 1.6M to about 2.4M, from about 1.7M to about 2.3M, from about 1.8M to about 2.2M, or from about 1.9M to about 2.1M. In other aspects, the AEX wash buffer comprises a salt concentration about 1M, about 1.1M, about 1.2M, about 1.3M, about 1.4M, about 1.5M, about 1.6M, about 1.7M, about 1.8M, about 1.9M, about 2.0M, about 2.1M, about 2.2M, about 2.3M, about 2.4M, about 2.5M, about 2.6M, about 2.7M, about 2.8M, about 2.9M, or 3.0M. In some aspects, the AEX wash buffer comprises a salt concentration about 2M.

### II.B.3. Multi-Modal Chromatography (AMC)

In some aspects, the chromatography resin comprises a mixed mode chromatography (“MMC”) resin. In some aspects, samples comprising EVs are purified by MMC after being purified by AEX. In some aspects, samples comprising EVs are purified by MMC before being purified by AEX. In some aspects, samples comprising EVs are purified by MMC after being purified by CEX. In some aspects, samples comprising EVs are purified by MMC before being purified by CEX. In some aspects, samples purified by AEX or CEX are processed by depth filtration before further being processed by MMC. In some aspects, adsorptive depth filter is used. In some aspects, an AEX-processed sample further processed by depth filtration is applied to MMC for purification.

Mixed mode chromatography employs chromatographic resins containing ligands possessing more than one type of functional groups. This unique property of mixed mode resin enables binding through multiple chromatographic modes in a single resin. Most resins in this class comprise a ligand containing a hydrophobic group (e.g. phenyl, benzyl, propyl, butyl, etc.) and a charged group (e.g. cation: sulfate, carboxylic acid, methyl carboxylic acid; or an anion: quaternary amine, diethylaminoethyl, diethylaminopropyl, or quaternary ammonium). However, some resins may also contain a hydrophilic group in place of the hydrophobic group, (e.g. silica, urea, polyethyleneimine, amino or amide groups, cyanopropyl, diol, or aminopropyl).

In some aspects, MMC resins comprise conventional chromatography ligands. In some aspects, the ligands are selected from tertiary amines, quaternary amines, diethaminoethyl, ceramic hydroxyapatite, ceramic fluoroapatite, butyl, hexyl, ether, hydroxyl, polypropylene glycol, phenyl, benzyl, sulfate, sulfopropyl, sulfobutyl, sulfoisobutyl, sulfoethyl, sulfonate, sulfonic acid, carboxymethyl, carboxylic acid, glutamic acid, aspartic acid, histidine, hydroxyl, phosphate ligands, and mixtures thereof. In some aspects, the chromatography ligands are formulated as CAPTO™ MMC, CAPTO™ adhere, CAPTO™ MMC ImpRes, CAPTO™ adhere ImpRes, CAPTO™ Core 700, or CAPTO™ Core 700, available from GE Healthcare; ESHMUNO® HCX, available from Merck Millapore; TOYOPEARL® MX-Trp-650M, available from Tosoh Bioscience; NUVIA™ CPRIME™, available from BioRad Laboratories; or CMM HYPERCEL™, HEA HYPERCEL™ or PPA HyperCel™, available from Pall Corporation; In some aspects, the MMC resin is a resin used in other types of chromatography (i.e., AEX, CEX, HIC, HCIC, etc.). In some aspects, the MMC resin is CMM HYPERCEL™, available from Pall Corporation.

In some aspects, the resins used in MMC comprise anion-exchange/reversed-phase (AEX/RP), cation-exchange/reversed phase (CEX/RP), anion-exchange/cation-exchange/reversed phase (AEX/CEX/RP), AEX/hydrophilic (AEX/HIIIC), CEX-hydrophilic (CEX/HILIC), or AEX/CEX hydrophilic (AEX/CEX/HILIC). An example of AEX/RP ligand is a hydrophobic, anionic ligand with hydrogen bonding that contains a quaternary amine, a phenyl group, and allows hydrogen bonding. An example of a CEX/RP ligand is a cationic ligand with hydrophobic binding that contain a secondary amine and is cationic over a wide pH range. Some mixed mode ligands are pH controllable, such as those containing 4-mercaptoethylpyridine ligands. The uncharged nitrogen in the pyridine ring becomes charged as pH decreases, resulting in a pH controllable mixed-mode ligand.

In some aspects, mixed mode ligands can be immobilized on the base matrix. In some aspects, the base matrix comprises membranes, monoliths, beaded resins, nanofibers, and/or other absorptive or convective media. In some aspects, the base matrix comprises cellulose, agarose, polystyrene derivatives, silica, methacrylate derivatives, glass, ceramic hydroxyapatite, PVDF, PTFE, polyethersulfone, polypropylene, polyethylene, acrylamide, a ceramic fluoroapatite, and/or any mixtures or derivatives thereof.

Mixed mode media comprising a single or plurality of ligands and a base matrix can be classified into four categories based on the arrangement of the ligand substrates on the base matrix. Type I media are mixtures of separation media, each with a single chemistry, packed to form a column. Type II media comprise substrates modified with a mixture of ligands having different functionalities, such as ion exchange, reverse phase, or hydrophilic phase properties. In Type III media, the functional ligands can be “embedded” in a hydrophobic chain, or in Type IV media, the hydrophobic chain can be “tipped” with the functional group. The mixed mode resins comprising a base matrix and one or more functional groups may be comprised of any of the types of media as described herein.

In some aspects, a MMC chromatography column is generated with the resin disclosed herein. The resin can be formed in a suspension, in slurry, or can be packed into a chromatography column.

In some aspects, the MMC chromatography column can further comprise conventional chromatography ligands selected from sulfate, tertiary amines, quaternary amines, carboxy methyl, carboxylic acids, diethaminoethyl, ceramic hydroxy apatite and ceramic fluoroapatite, or any combination thereof. In some aspects, conventional chromatography ligands can be formulated as CAPTO MMC™ or CAPTO ADHERE™ available from GE Healthcare Life Sciences; TOYOPEARL MX-TRP™ available from Tosoh Bioscience; HYPERCEL™ STAR AX available from Pall Corporation; NUVIA™ CPRIME™ available from BioRad; or ESHMUNO™ HCX available from EMD Millipore.

In some aspects, hydrophobic, hydrophilic, and/or ionic mixed mode ligands and the conventional chromatography ligands are displayed on the same resin. For example, the hydrophobic, hydrophilic, and/or ionic mixed mode ligands and the conventional chromatography ligands are immobilized on the base matrix (e.g., membranes, monoliths, beaded resins, nanofibers, and other absorptive or convective media). In some aspects, hydrophobic, hydrophilic, and/or ionic mixed mode ligands and chromatographic ligands are intermixed. In some aspects, hydrophobic, hydrophilic, and/or ionic mixed mode ligands and chromatographic ligands are displayed on separate layers.

In some aspects, mixed mode media comprises hydrophobic ligands. Hydrophobic ligands can be used to purify EVs based on their interaction with a nonpolar surface on EVs, an amphiphilic phospholipid bilayer membrane with embedded transmembrane proteins or an outer bilayer surface that is associated with a variety of proteins, nucleic acids, lipids, and carbohydrates. Hydrophobic groups of the biomolecules that are sufficiently exposed to the surface allow interaction with hydrophobic ligands. In some aspects, the hydrophobic ligands can be hydrophobic alkyl or aryl groups. In some aspects, the hydrophobic alkyl or aryl groups are selected from phenyl, ethyl, methyl, pentyl, heptyl, benzyl, octyl, butyl, hexyl, ether, hydroxyl, polypropylene glycol, and the like.

In some aspects, mixed mode media comprises hydrophilic ligands. Hydrophilic ligands can be used to purify EVs via flow through mode, or to purify desired subgroups of EVs. The amphiphilic surface of the EVs may not bind to the hydrophilic ligands of the column, while polar impurities or proteins in the sample interact with the hydrophilic ligands. In some aspects, the hydrophilic ligands comprise silica, urea, amino groups, amide groups, polyethyleneimine, cyanopropyl, diol, aminopropyl, and/or zwitterions such as sulnfoalkylbetaine.

In some aspects, mixed mode media comprises CEX ligands.

In some aspects, mixed mode media comprises AEX ligands.

In some aspects, MMC chromatography is performed in a bind-elute mode. In some aspects, MMC chromatography is performed in a weak-partitioning mode.

According to the present disclosure, additional chromatography process can be used in addition to the chromatography processes disclosed herein (e.g., CEX-AEX or CEX-AEX-MMC). In some aspects, the additional chromatography can be used instead of the MMC process. In other aspects, the additional chromatography can be used in addition to the CEX, AEX, and MMC. In some aspects, a CEX, such as a CMM HYPERCEL™ chromatography column, is operated in series with a MMC, such as a CaptoCore700™ column, operated in flow through mode. In some aspects, a CEX-MMC is operated in series in flow-through mode. In some aspects, a MMC-CEX is operated in series in flow-through mode.

In some aspects, the present method further comprises hydrophobic interaction chromatography (“HIC”). In some aspects, the present method further comprises hydrophobic charge induction chromatography (“HCIC”).

The HIC or HCIC uses hydrophobic ligands attached to a base matrix. In some aspects the base matrix comprises membranes, monoliths, beaded resins, nanofibers, and/or other absorptive or convective media. In some aspects, the base matrix comprises cellulose, agarose, polystyrene derivatives, silica, methacrylate derivatives, glass, ceramic hydroxyapatite, PVDF, PTFE, polyethersulfone, polypropylene, polyethylene, acrylamide, a ceramic fluoroapatite, and/or any mixtures or derivatives thereof.

Purification of EVs by hydrophobic ligands is based on the interaction between the ligands and a nonpolar surface on EVs, an amphiphilic phospholipid bilayer membrane with embedded transmembrane proteins or an outer bilayer surface that is associated with a variety of proteins, nucleic acids, lipids, and carbohydrates. Hydrophobic groups of the biomolecules that are sufficiently exposed to the surface can interact with hydrophobic ligands.

In some aspects, hydrophobic ligands that can be used for the present invention include ligands comprising hydrophobic alkyl and/or aryl groups. In some aspects the hydrophobic alkyl or aryl group are selected from phenyl, ethyl, methyl, pentyl, heptyl, benzyl, octyl, butyl, hexyl, ether, hydroxyl, polypropylene glycol, and mixtures thereof.

In some aspects, the salt concentration of the MMC loading buffer, elution buffer, and/or wash buffer is at least about 100 mM, at least about 200 mM, at least about 300 mM, at least about 400 mM, at least about 500 mM, at least about 600 mM, at least about 700 mM, at least about 800 mM, at least about 900 mM, at least about 1M, at least about 1.1M, at least about 1.2M, at least about 1.3M, at least about 1.4M, at least about 1.5M, at least about 1.6M, at least about 1.7M, at least about 1.8M, at least about 1.9M, at least about 2.0M, at least about 2.1M, at least about 2.2M, at least about 2.3M, at least about 2.4M, or at least about 2.5M. In other aspects, the sale concentration of the MMC loading buffer is between about 10 mM and about 5M, between about 100 mM and about 5M, between about 100 mM and about 4M, between about 100 mM and about 3M, between about 200 mM and about 5M, between about 300 mM and about 4M, between about 400 mM and about 3M, between about 500 mM and about 2M, between about 1M and about 3M, between about 1 mM and about 2M, between about 800 mM and about 2M, between about 900 mM and about 2.5M, or between about 1.5M and about 2.5M. In some aspects, the salt concentration of the MMC loading buffer and wash buffer is about 1M.

In some aspects, the pH of the MMC loading buffer and/or wash buffer is about 7.5.

### II.B.4. Affinity Chromatography

In some aspects, the chromatography resin comprises an affinity chromatography resin. Affinity chromatography separates target molecules from non-target molecules in a mixture by utilizing highly specific binding between the affinity chromatography resin and the target molecule. In some aspects, the affinity chromatography resin interacts with the EVs, e.g., exosomes. In some aspects, the affinity chromatography resin comprises a binding moiety, wherein the binding moiety interacts with a target protein on the surface of the EV, e.g., exosome. In some aspects, the binding moiety interacts with a scaffold protein. In some aspects, the binding moiety interacts with PTGFRN. In some aspects, the binding moiety interacts with a fragment of PTGFRN. In some aspects, the binding moiety interacts with a Scaffold X protein. In some aspects, the chromatography resin comprises a pseudo affinity chromatography resin.

### II.C. Buffers

Certain aspects of the present disclosure are directed to methods of preparing purified EVs, e.g., exosomes, from a sample comprising EVs, e.g., exosomes, and one or more nucleic acid molecules, comprising: (i) contacting the sample with a chromatography resin and (ii) contacting the chromatography resin with a wash buffer, e.g., a wash buffer comprising MgCL₂, CaCl₂, KCl, and/or NaCl, wherein the wash buffer does not comprise a nuclease, and wherein the (ii) washing follows the (i) contacting. Some aspects of the present disclosure are directed to methods of reducing the concentration of residual nucleic acid molecule in a sample comprising EVs, e.g., exosomes, comprising (i) contacting the sample with a chromatography resin and (ii) contacting the chromatography resin with a wash buffer, e.g., a wash buffer comprising MgCL₂, CaCl₂, KCl, and/or NaCl, wherein the wash buffer does not comprise a nuclease, and wherein the (ii) washing follows the (i) contacting. In some aspects, the sample is contacted with the chromatography resin in a loading buffer, e.g., a loading buffer comprising the sample is contacted with the chromatography resin.

The loading buffer used herein can comprise any one or more elements in addition to the EVs, e.g., exosomes, and the one or more nucleic acid molecules. In certain aspects, the loading buffer comprises a salt. In some aspects, the loading buffer comprises a salt concentration of less than about 1.2 M (e.g., less than about 1.1 M, less than about 1.0 M, less than about 0.9 M, less than about 0.8 M, less than about 0.7 M, less than about 0.6 M, less than about 0.5 M, less than about 0.4 M, less than about 0.3 M, less than about 0.2 M, less than about 0.1 M.

In some aspects, the loading buffer comprises a salt concentration from at least about 0.01 M to at least about 1.0 M. In some aspects, the salt concentration of the loading buffer is at least about 0.01 M, at least about 0.05 M, at least about 0.1 M, at least about 0.15 M, at least about 0.2 M, at least about 0.25 M, at least about 0.3 M, at least about 0.35 M, at least about 0.4 M, at least about 0.45 M, at least about 0.5 M, at least about 0.55 M, at least about 0.6 M, at least about 0.65 M, at least about 0.7 M, at least about 0.75 M, at least about 0.8 M, at least about 0.85 M, at least about 0.9 M, at least about 0.95 M, or at least about 1.0 M.

In some aspects, the salt of the loading buffer is selected from NaCl, KCl, PO₄, CaCl₂, MgCl₂, and any combination thereof. In some aspects, the salt of the loading buffer is selected from NaCl, KCl, KPO₄, NaPO₄, CaCl₂, Mg₂SO₄, ZnCl₂, MnCl₂, MnSO₄, NaSCN, KSCN, LiCl, and any combination thereof. In certain aspects, the loading buffer comprises NaCl. In certain aspects, the loading buffer comprises KCl. In certain aspects, the loading buffer comprises KPO₄. In certain aspects, the loading buffer comprises CaCl₂. In certain aspects, the loading buffer comprises NaPO₄. In certain aspects, the loading buffer comprises Mg₂SO₄. In certain aspects, the loading buffer comprises ZnCl₂. In certain aspects, the loading buffer comprises MnCl₂. In certain aspects, the loading buffer comprises MnSO₄. In certain aspects, the loading buffer comprises NaSCN. In certain aspects, the loading buffer comprises KSCN. In certain aspects, the loading buffer comprises LiCl. In certain aspects, the loading buffer comprises MgCl₂. In some aspects, the loading buffer comprises a salt, wherein the salt comprises an anion selected from F⁻, SO₄²⁻, HPO₄²⁻, PO₄²⁻, acetate, Cl⁻, NO₃⁻, Br⁻, ClO₃⁻, SCN⁻ or any combination thereof. In some aspects, the loading buffer comprises HEPES, BES, Bicine, MES, MOPS, PIPES, acetate, carbonate, citrate, bicarbonate buffer, aspartic acid, glutamic acid, and any combination thereof. In some aspects, the loading buffer comprises a salt, wherein the salt comprises a cation selected from NH₄⁺, K⁺, Na⁺, Li²⁺, Mg²⁺, Ca²⁺, guanidinium, and any combination thereof. In some aspects, the loading buffer comprises a buffer selected from the group consisting of a Imidazole, Tris, TAPS, BisTRIS, arginine, histidine, lysine buffer, and any combination thereof

In some aspects the loading buffer comprises at least about 0.01 M NaCl, at least about 0.05 M NaCl, at least about 0.1 M NaCl, at least about 0.15 M NaCl, at least about 0.2 M NaCl, at least about 0.25 M NaCl, at least about 0.3 M NaCl, at least about 0.35 M NaCl, at least about 0.4 M NaCl, at least about 0.45 M NaCl, at least about 0.5 M NaCl, at least about 0.55 M NaCl, at least about 0.6 M NaCl, at least about 0.65 M NaCl, at least about 0.7 M NaCl, at least about 0.75 M NaCl, at least about 0.8 M NaCl, at least about 0.85 M NaCl, at least about 0.9 M NaCl, at least about 0.95 M NaCl, at least about 1 M NaCl, at least about 1.1 M NaCl, 1.2 M NaCl, at least about 1.3 M NaCl, at least about 1.4 M NaCl, at least about 1.5 M NaCl, at least about 1.6 M NaCl, at least about 1.7 M NaCl, at least about 1.8 M NaCl, at least about 1.9 M NaCl, at least about 2 M NaCl. In certain aspects, the loading buffer comprises at least about 0.55 M NaCl.

In some aspects, the method comprises:
a. contacting the sample with a chromatography resin, wherein the EVs, e.g., exosomes, associate with the chromatography resin;
b. applying a wash buffer to the chromatography resin, wherein the wash buffer comprises MgCl₂, CaCl₂, KCl, and/or NaCl, and wherein the wash buffer does not comprise a nuclease;
c. applying a second wash buffer to the chromatography resin, wherein the second wash buffer does not comprise a nuclease;
d. applying an elution buffer to the chromatography resin; and
e. collecting the eluent, wherein the eluent comprises the EVs, e.g., exosomes.

In some aspects, the method comprises:
a. contacting the sample with a chromatography resin, wherein the EVs, e.g., exosomes, associate with the chromatography resin;
b. applying a wash buffer to the chromatography resin, wherein the wash buffer comprises MgCl₂, and wherein the wash buffer does not comprise a nuclease;
c. applying a second wash buffer to the chromatography resin, wherein the second wash buffer comprises NaCl, and wherein the second wash buffer does not comprise a nuclease;
d. applying an elution buffer to the chromatography resin; and
e. collecting the eluent, wherein the eluent comprises the EVs, e.g., exosomes.

In some aspects, the method comprises:
a. contacting the sample with a chromatography resin, wherein the EVs, e.g., exosomes, associate with the chromatography resin;
b. applying a wash buffer to the chromatography resin, wherein the wash buffer comprises MgCl₂, wherein the concentration of Mg²⁺ in the wash buffer is about 350 mM; and wherein the wash buffer does not comprise a nuclease;
c. applying a second wash buffer to the chromatography resin, wherein the second wash buffer comprises about 800 mM NaCl, and wherein the second wash buffer does not comprise a nuclease;
d. applying an elution buffer to the chromatography resin; and
e. collecting the eluent, wherein the eluent comprises the EVs, e.g., exosomes.

In some aspects, the method comprises:
a. contacting the sample with a chromatography resin, wherein the EVs, e.g., exosomes, associate with the chromatography resin;
b. applying a wash buffer to the chromatography resin, wherein the wash buffer comprises NaCl, and wherein the wash buffer does not comprise a nuclease;
c. applying a second wash buffer to the chromatography resin, wherein the second wash buffer comprises MgCl₂, and wherein the second wash buffer does not comprise a nuclease;
d. applying an elution buffer to the chromatography resin; and
e. collecting the eluent, wherein the eluent comprises the EVs, e.g., exosomes.

In some aspects, the method comprises:
a. contacting the sample with a chromatography resin, wherein the EVs, e.g., exosomes, associate with the chromatography resin;
b. applying a wash buffer to the chromatography resin, wherein the wash buffer comprises about 800 mM NaCl; and wherein the wash buffer does not comprise a nuclease;
c. applying a second wash buffer to the chromatography resin, wherein the second wash buffer comprises MgCl₂, wherein the concentration of Mg²⁺ in the wash buffer is about 350 mM, and wherein the second wash buffer does not comprise a nuclease;
d. applying an elution buffer to the chromatography resin; and
e. collecting the eluent, wherein the eluent comprises the EVs, e.g., exosomes.

In some aspects, the methods disclosed herein further comprise (iii) eluting the EVs from the chromatography resin by contacting the chromatography resin with an elution buffer, wherein (iii) occurs after (ii) contacting the chromatography resin with a wash buffer, e.g., a wash buffer comprising MgCl₂, CaCl₂, KCl, and/or NaCl. In some aspects, the elution buffer releases one or more EVs from the chromatography resin. In certain aspects, the method further comprises collecting an eluent after contacting the chromatography resin with the elution buffer. In some aspects, the eluent comprises one or more EVs.

In some aspects, the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M. In some aspects, the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M NaCl. In certain aspects, the elution buffer comprises at least about 1.2 M NaCl.

In some aspects, the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M. In some aspects, the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M KCl. In certain aspects, the elution buffer comprises at least about 1.2 M KCl.

### II.D. Additional Purification Steps

In some aspects, the sample comprising the EVs, e.g., exosomes, and the one or more nucleic acid molecule is subjected to one or more additional purification step. In some aspects, the sample is subject to one or more additional purification step prior to (i) contacting the sample with a chromatography resin, and (ii) contacting the chromatography resin with a wash buffer, e.g., a wash buffer comprising MgCl₂, CaCl₂, and/or NaCl. In some aspects, the sample is subject to one or more additional purification step after to (i) contacting the sample with a chromatography resin, and (ii) contacting the chromatography resin with a wash buffer, e.g., a wash buffer comprising MgCl₂, CaCl₂, and/or NaCl. In some aspects, the sample is subject to one or more additional purification step both prior to and after (i) contacting the sample with a chromatography resin, and (ii) contacting the chromatography resin with a wash buffer, e.g., a wash buffer comprising MgCl₂, CaCl₂, and/or NaCl.

In some aspects, one or more filtration steps are added before, after, or between the chromatographic purification steps. For example, adsorptive depth filtrations step can be added before, between, or after chromatographic steps: (i) Filtration-CEX-AEX-MMC; (ii) CEX-Filtration-AEX-MMC; (iii) CEX-AEX-Filtration-MMC; (iv) CEX-AEX-MMC-filtration; (v) Filtration-CEX-MMC-AEX; (vi) CEX-Filtration-MMC-AEX; (vii) CEX-MMC-Filtration-AEX; (viii) CEX-MMC-AEX-Filtration; (ix) Filtration-AEX-CEX-MMC; (x) AEX-Filtration-CEX-MMC; (xi) AEX-CEX-Filtration-MMC; (xii) AEX-CEX-MMC-Filtration; (xiii) Filtration-AEX-MMC-CEX; (xiv) AEX-Filtration-MMC-CEX; (xv) AEX-MMC-Filtration-CEX; (xvi) AEX-MMC-CEX-Filtration; (xvii) Filtration-MMC-CEX-AEX; (xvii) MMC-Filtration-CEX-AEX; (xvii) MMC-CEX-Filtration-AEX; (xvii) MMC-CEX-AEX-Filtration; (xviii) Filtration-MMC-AEX-CEX; (xix) MMC-Filtration-AEX-CEX; (xx) MMC-AEX-Filtration-CEX; or (xxi) MMC-AEX-CEX-Filtration. Any one of filtration described herein can be used for the filtration. In some aspects, the present method comprises: (1) Filtration(1)-CEX-Filtration(2)-AEX-MMC; (2) Filtration(1)-CEX-AEX-Filtration(2)-MMC; (3) Filtration(1)-CEX-AEX-MMC-Filtration(2); (4) CEX-Filtration(1)-AEX-Filtration(2)-MMC; (5) CEX-Filtration(1)-AEX-MMC-Filtration(2); (6) CEX-AEX-Filtration(1)-MMC-Filtration(2); (7) Filtration(1)-CEX-Filtration(2)-MMC-AEX; (8) Filtration(1)-CEX-MMC-Filtration(2)-AEX; (9) Filtration(1)-CEX-MMC-AEX-Filtration(2); (10) CEX-Filtration(1)-MMC-Filtration(2)-AEX; (11) CEX-Filtration(1)-MMC-AEX-Filtration(2); (12) CEX-MMC-Filtration(1)-AEX-Filtration(2); (13) Filtration(1)-AEX-Filtration(2)-CEX-MMC; (14) Filtration(1)-AEX-CEX-Filtration(2)-MMC; (15) Filtration(1)-AEX-CEX-MMC-Filtration(2); (16) AEX-Filtration(1)-CEX-Filtration(2)-MMC; (17) AEX-Filtration(1)-CEX-MMC-Filtration(2); (18) AEX-CEX-Filtration(1)-MMC-Filtration(2); (19) Filtration(1)-AEX-Filtration(2)-MMC-CEX; (20) Filtration(1)-AEX-MMC-Filtration(2)-CEX; (21) Filtration(1)-AEX-MMC-CEX-Filtration(2); (22) AEX-Filtration(1)-MMC-Filtration(2)-CEX; (23) AEX-Filtration(1)-MMC-CEX-Filtration(2); (24) AEX-MMC-Filtration(1)-CEX-Filtration(1); (25) Filtration(1)-MMC-Filtration(2)-CEX-AEX; (26) Filtration(1)-MMC-CEX-Filtration(2)-AEX; (27) Filtration(1)-MMC-CEX-AEX-Filtration(2); (28) MMC-Filtration(1)-CEX-Filtration(2)-AEX; (29) MMC-Filtration(1)-CEX-AEX-Filtration(2); (30) MMC-CEX-Filtration(1)-AEX-Filtration(2); (31) Filtration(1)-MMC-Filtration(2)-AEX-CEX; (32) Filtration(1)-MMC-AEX-Filtration(2)-CEX; (33) Filtration(1)-MMC-AEX-CEX-Filtration(2); (34) MMC-Filtration(1)-AEX-Filtration(2)-CEX; (35) MMC-Filtration(1)-AEX-CEX-Filtration(2); or (36) MMC-AEX-Filtration(1)-CEX-Filtration(2). In some aspects, Filtration (1) is the same as Filtration (2). In other aspects, Filtration (1) is different from Filtration (2). In other aspects, any filtration prior to the CEX process has a bigger filter size compared to a filter size of filtration after the CEX process. In some aspects, the filter size of the filtrations is reduced in or after the CEX process. In some aspects, the filter size prior to the CEX process is bigger than about 0.14 micron, about 0.16 micron, about 0.18 micron, about 0.2 micron, about 0.25 micron, about 0.3 micron, about 0.35 micron, about 0.4 micron, about 0.45 micron, about 0.5 micron, about 0.55 micron, about 0.6 micron, about 0.65 micron, or about 0.7 micron. In other aspects, the filter size of the filtrations in or after the CEX process is smaller than about 0.25 micron, about 0.22 micron, about 0.2 micron, about 0.18 micron, about 0.16 micron, or about 0.14 micron. In some aspects, the method of the disclosure comprises AEX-Filtration-CEX-MMC.

In some aspects, the present method comprises: (1) Filtration(1)-CEX-Filtration(2)-AEX-Filtration(3)-MMC; (2) Filtration(1)-CEX-Filtration(2)-AEX-MMC-Filtration(3); (3) Filtration(1)-CEX-AEX-Filtration(2)-MMC-Filtration(3); (4) CEX-Filtration(1)-AEX-Filtration(2)-MMC-Filtration(3); (5) Filtration(1)-CEX-Filtration(2)-MMC-Filtration(3)-AEX; (6) Filtration(1)-CEX-Filtration(2)-MMC-AEX-Filtration(3); (7) Filtration(1)-CEX-MMC-Filtration(2)-AEX-Filtration(3); (8) CEX-Filtration(1)-MMC-Filtration(2)-AEX-Filtration(3); (9) Filtration(1)-AEX-Filtration(2)-CEX-Filtration(3)-MMC; (10) Filtration(1)-AEX-Filtration(2)-CEX-MMC-Filtration(3); (11) Filtration(1)-AEX-CEX-Filtration(2)-MMC-Filtration(3); (12) AEX-Filtration(1)-CEX-Filtration(2)-MMC-Filtration(3); (13) Filtration(1)-AEX-Filtration(2)-MMC-Filtration(3)-CEX; (14) Filtration(1)-AEX-Filtration(2)-MMC-CEX-Filtration(3); (15) Filtration(1)-AEX-MMC-Filtration(2)-CEX-Filtration(3); (16) AEX-Filtration(1)-MMC-Filtration(2)-CEX-Filtration(3); (17) Filtration(1)-MMC-Filtration(2)-CEX-Filtration(3)-AEX; (18) Filtration(1)-MMC-Filtration(2)-CEX-AEX-Filtration(3); (19) Filtration(1)-MMC-CEX-Filtration(2)-AEX-Filtration(3); (20) MMC-Filtration(1)-CEX-Filtration(2)-AEX-Filtration(3); (21) Filtration(1)-MMC-Filtration(2)-AEX-Filtration(3)-CEX; (22) Filtration(1)-MMC-Filtration(2)-AEX-CEX-Filtration(3); (23) Filtration(1)-MMC-AEX-Filtration(2)-CEX-Filtration(3); (24) MMC-Filtration(1)-AEX-Filtration(2)-CEX-Filtration(3). In some aspects, the method comprises Filtration (1)-AEX-Filtration (2)-CEX-MMC-Filtration (3). In other aspects, any filtration prior to the CEX process has a bigger filter size compared to a filter size of filtration after the CEX process. In some aspects, the filter size of the filtrations is reduced in or after the CEX process. In some aspects, the filter size prior to the CEX process is bigger than about 0.25 micron, about 0.3 micron, about 0.35 micron, about 0.4 micron, about 0.45 micron, about 0.5 micron, about 0.55 micron, about 0.6 micron, about 0.65 micron, or about 0.7 micron. In other aspects, the filter size of the filtrations in or after the CEX process is smaller than about 0.25 micron, about 0.22 micron, about 0.2 micron, about 0.18 micron, about 0.16 micron, or about 0.14 micron.

In some aspects, the present method comprises: (1) Filtration(1)-CEX-Filtration(2)-AEX-Filtration(3)-MMC-Filtration(4); (2) Filtration(1)-CEX-Filtration(2)-MMC-Filtration(3)-AEX-Filtration(4); (3) Filtration(1)-AEX-Filtration(2)-CEX-Filtration(3)-MMC-Filtration(4); (4) Filtration(1)-AEX-Filtration(2)-MMC-Filtration(3)-CEX-Filtration(4); (5) Filtration(1)-MMC-Filtration(2)-CEX-Filtration(3)-AEX-Filtration(4); or (6) Filtration(1)-MMC-Filtration(2)-AEX-Filtration(3)-CEX-Filtration(4). In other aspects, any filtration prior to the CEX process has a bigger filter size compared to a filter size of filtration in or after the CEX process. In some aspects, the filter size of the various filtrations is reduced in or after the CEX process. In some aspects, the filter size prior to the CEX process is bigger than about 0.25 micron, about 0.3 micron, about 0.35 micron, about 0.4 micron, about 0.45 micron, about 0.5 micron, about 0.55 micron, about 0.6 micron, about 0.65 micron, or about 0.7 micron. In other aspects, the filter size of the filtrations in or after the CEX process is smaller than about 0.25 micron, about 0.22 micron, about 0.2 micron, about 0.18 micron, about 0.16 micron, or about 0.14 micron. In some aspects, the present filtration useful in the process is a sterile filtration. One or more sterile filtrations can be performed within the present methods. In some aspects, at least one, at least two, at least three, at least four, at least five, at least six, at least seven, at least eight, at least nine, at least ten, at least 11, at least 12, at least 13, at least 14, or at least 15 filtrations can be introduced in the present methods. In some aspects, a sterile filtration can be introduced between two chromatographies. In some aspects, filtration can be used right after the harvest. In other aspects, filtration can be used right before formulation.

### III. Samples Comprising EVs

Samples comprising EVs useful for the present methods can be obtained from a various in vitro cell culture or a harvest or a supernatant of the cell culture. In some aspects, the sample comprising EVs can be obtained from a mammalian cell, a bacterial cell, a eukaryotic cell, a prokaryotic cell, a plant cell, an insect cell, or any combination thereof. In some aspects, the sample comprising EVs can be obtained from a mammalian cell. In some aspects, the sample comprising EVs can be obtained from a HEK cell culture. In some aspects, the sample comprising EVs can be a cell culture comprising cells producing EVs.

The present disclosure provides a method for preparing EVs, which can be implemented to purify EVs in a large scale. In some aspects, the method can be applied to purify EVs from a sample with a volume larger than about 1 L, about 5 L, about 10 L, about 15 L about 20 L, about 25 L, about 50 L, about 100 L, about 200 L, about 250 L, about 300 L, about 400 L, about 500 L, about 600 L, about 700 L, about 800 L, about 900 L, about 1000 L, or about 2000 L. In some aspects, the method can be applied to purify EVs from a sample with a volume of about 400 L. In some aspects, the method can be applied to purify EVs from a sample with a volume of about 500 L. In some aspects, the method can be applied to purify EVs from a sample with a volume of about 600 L. In some aspects, the method can be applied to purify EVs from a sample with a volume larger than about 100 L. In some aspects, the method can be applied to purify EVs from a sample with a volume larger than about 200 L. In some aspects, the method can be applied to purify EVs from a sample with a volume larger than about 300 L. In some aspects, the method can be applied to purify EVs from a sample with a volume larger than about 700 L. In some aspects, the method can be applied to purify EVs from a sample with a volume larger than about 1000 L. In some aspects, the method can be applied to purify EVs from a sample with a volume larger than about 1500 L. In some aspects, the method can be applied to purify EVs from a sample with a volume larger than about 2000 L.

In some aspects, the cell culture media useful for the present methods comprises 3D suspension culture comprising high-depth chemically defined media. In some aspects, the method of the present disclosure includes continuous manufacturing processes. In some aspects, the methods comprise continuous manufacturing processes at high cell density (e.g., at least about 50×10⁶ cells/ml, at least about 60×10⁶ cells/ml, at least about 70×10⁶ cells/ml, at least about 80×10⁶ cells/ml, at least about 90×10⁶ cells/ml, at least about 100×10⁶ cells/ml, at least about 110×10⁶ cells/ml, at least about 120×10⁶ cells/ml, at least about 130×10⁶ cells/ml, at least about 140×10⁶ cells/ml, at least about 150×10⁶ cells/ml, at least about 200×10⁶ cells/ml, at least about 250×10⁶ cells/ml, at least about 300×10⁶ cells/ml, at least about 350×10⁶ cells/ml, or at least about 400×10⁶ cells/ml, e.g., 40×10⁶ to 200×10⁶ cell/ml, e.g., 50×10⁶ to 170×10⁶ cell/ml, e.g., 50×10⁶ to 150×10⁶ cell/ml).

In some aspects, each sample has a volume of about 500 L and the 500 L volume sample goes through the purification step (e.g., CEX; AEX; Affinity; CEX and AEX; CEX, AEX, and MMC; or any other combinations) as described herein. In other aspects, the total amount of sample that goes through the purification step for each batch is at least about 5,000 L, at least about 6,000 L, at least about 7,000 L, at least about 8,000 L, at least about 9,000 L, at least about 10,000 L, at least about 11,000 L, at least about 12,000 L, at least about 13,000 L, at least about 14,000 L, or at least about 15,000 L. In other aspects, the total amount of sample that goes through the purification step for each batch is at least about 10,000 L. In other aspects, the total amount of sample that goes through the purification step for each batch is at least about 15,000 L. In other aspects, the total amount of sample that goes through the purification step for each batch is at least about 20,000 L.

In some aspects, the EVs that can be purified by the present methods comprise naturally-occurring EVs, e.g., exosomes. In some aspects, the EVs that can be purified by the present methods comprise engineered EVs, e.g., exosomes. In some aspects, the EVs that can be purified by the present methods comprise surface-engineered EVs, e.g., exosomes. In some aspects, the EVs that can be purified by the present methods comprise engineered EVs, e.g., exosomes that contain one or more (heterologous) moieties in the lumen of the EVs, e.g., exosomes (e.g., encapsulated in the EVs). In some aspects, the EVs that can be purified by the present methods comprise engineered EVs that contain one or more (heterologous) moieties linked to a moiety on the exterior surface of the EVs. In some aspects, the EVs that can be purified by the present methods comprise engineered EVs that contain one or more (heterologous) moieties linked to a moiety on the luminal surface of the EVs.

In other aspects, the EVs from the producer cell can have a longest dimension of from about 20 to about 1000 nm. In some aspects, the EVs from the producer cell can have a longest dimension of from about 20 to about 900 nm, from about 20 to about 800 nm, from about 20 to about 700 nm, from about 20 to about 600 nm, from about 20 to about 500 nm, from about 20 to about 400 nm, from about 20 to about 350 nm, from about 20 to about 300 nm, from about 20 to about 290 nm, from about 20 to about 280 nm, from about 20 to about 270 nm, from about 20 to about 260 nm, from about 20 to about 250 nm, from about 20 to about 240 nm, from about 20 to about 230 nm, from about 20 to about 220 nm, from about 20 to about 210 nm, from about 20 to about 200 nm, from about 20 to about 190 nm, from about 20 to about 180 nm, from about 20 to about 170 nm, about 20 to about 160 nm, from about 20 to about 150 nm, from about 20 to about 140 nm, about 20 to about 130 nm, from about 20 to about 120 nm, In some aspects, the EVs from the producer cell can have a longest dimension of from about 20 to about 110 nm, from about 20 to about 100 nm, from about 20 to about 90 nm, In some aspects, the EVs from the producer cell can have a longest dimension of from about 20 to about 80 nm, from about 20 to about 70 nm, from about 20 to about 60 nm, from about 20 to about 50 nm, from about 20 to about 40 nm, from about 20 to about 30 nm, from about 30 to about 300 nm, from about 30 to about 290 nm, from about 30 to about 280 nm, from about 30 to about 270 nm, from about 30 to about 260 nm, from about 30 to about 250 nm, from about 30 to about 240 nm, from about 30 to about 230 nm, from about 30 to about 220 nm, about 30 to about 210 nm, from about 30 to about 200 nm, from about 30 to about 190 nm, from about 30 to about 180 nm, from about 30 to about 170 nm, from about 30 to about 160 nm, from about 30 to about 150 nm, from about 30 to about 140 nm, from about 30 to about 130 nm, from about 30 to about 120 nm, from about 30 to about 110 nm, from about 30 to about 100 nm, from about 30 to about 90 nm, from about 30 to about 80 nm, from about 30 to about 70 nm, or from about 30 to about 60 nm.

In some aspects, EV membranes comprise lipids and/or fatty acids. In some aspects, EV membranes comprise phospholipids, glycolipids, fatty acids, sphingolipids, phosphoglycerides, sterols, cholesterols, and/or phosphatidylserines. In some of these aspects, EV membranes further comprise one or more polypeptides and/or one or more polysaccharides, such as glycan.

In some aspects, EV membranes comprise one or more molecules derived from the producer cell. In some aspects, EVs can be generated in a cell culture system and isolated from the producer cell. In some aspects, EVs can be generated from a perfusion cell culture. In some aspects, EVs can be generated from a batch cell culture. In some aspects, EVs can be generated from a fed batch cell culture. In some aspects, EVs can be generated from suspension or adherent cells. In some aspects, EVs can be generated from a HEK293 cell, a CHO cell, a BHK cell, a PERC6 cell, a Vero cell, a HeLa cell, a sf9 cell, a PC12 cell, a mesenchymal stem cell, a human donor cell, a stem cell, a dendritic cell, an antigen presenting cell, an induced pluripotent stem cell (IPC), a differentiated cell, bacteria, Streptomyces, Drosophila, Xenopus oocytes, Escherichia coli, Bacillus subtilis, yeast, S. cerevisiae, Picchia pastoris, filamentous fungi, Neurospora crassa, and/or Aspergillus nidulans. In some aspects, the producer cell is a HEK293 cell. The process of EV generation would be generally applicable to bioreactor formats including AMBR, shake flasks, SUBs, Waves, Applikons, stirred tanks, CSTRs, adherent cell culture, hollow fibers, iCELLis, microcarriers, and other methods known to those of skill in the art.

The present disclosure also includes extracellular vesicles (EVs) produced by a cell line. The production of extracellular vesicles and maintenance of cell culture conditions are important to maintain viable cell density of a cell culture process and consistently produce high-quality extracellular vesicles over the full length of a cell culture process. In some aspects, the EVs purified by the present methods are produced in a bioreactor. In some aspects, the EVs purified by the present methods are produced in a single-use bioreactor. In some aspects, the EVs purified by the present methods are produced in a perfusion bioreactor. In some aspects, the EVs purified by the present methods are produced in an alternating tangential flow filtration (ATF) perfusion bioreactor. In some aspects, the EVs purified by the present methods are produced in a tangential flow filtration (TFF) perfusion bioreactor. In some aspects, the EVs purified by the present methods are produced in a bioreactor at a viable cell density (VCD) of about 1×10⁶ cells/mL, about 5×10⁶ cells/mL, about 10×10⁶ cells/mL, about 20×10⁶ cells/mL, about 30×10⁶ cells/mL, about 40×10⁶ cells/mL, about 50×10⁶ cells/mL, or about 60×10⁶ cells/mL. In some aspects, the EVs purified by the present methods are produced in a bioreactor at a viable cell density (VCD) of about 60×10⁶ cells/mL. In some aspects, the EVs purified by the present methods are produced in a bioreactor at a viable cell density (VCD) of about 50×10⁶ cells/mL. In some aspects, the EVs purified by the present methods are produced in a bioreactor at a viable cell density (VCD) of from about 0 to about 60×10⁶ cells/mL, from about 1×10⁶ cells/mL to about 60×10⁶ cells/mL, from about 40×10⁶ cells/mL to about 60×10⁶ cells/mL, or from about 50×10⁶ cells/mL to about 60×10⁶ cells/mL.

In some aspects, the EVs purified by the present methods are produced in a bioreactor for about 5 days, about 10 days, about 15 days, about 20 days, about 25 days, or about 30 days. In some aspects, the EVs purified by the present methods are produced in a bioreactor for about 1-30 days, about 1-45 days, about 1-60 days, about 1-10 days, about 5-10 days, or about 1-25 days. In some aspects, the EVs purified by the present methods are produced in a bioreactor for about 1-30 days.

In some other aspects, EVs are modified by altering components of the membrane of the EV. In some of these aspects, EVs are modified by altering the protein, lipid and/or glycan content of the membrane. In other aspects, EVs are engineered to express a scaffold moiety, e.g., Scaffold X, Scaffold Y, or any other moieties. In some aspects, EVs are engineered to express a higher number of one or more proteins naturally expressed on the surface of producer cells or EVs.

In some aspects, the producer cells naturally contain one or more polypeptides, and EVs derived from the producer cell also contain the one or more polypeptides. In some aspects, the producer cells are modified to contain one or more polypeptides. In some aspects, the modification comprises modulating expression of the one or more polypeptides through use of agents that alter endogenous gene expression. In some aspects, the modification comprises modulating expression of the one or more polypeptides through introduction of expression constructs or mRNAs that encode the one or more polypeptides. In some aspects, EVs produced by these cells include the one or more polypeptides as a payload.

In some aspects, the EV protein is Scaffold X. In some aspects, EVs comprise one or more polypeptides on their surface. In some aspects, the one or more polypeptides can be CD47, CD55, CD49, CD40, CD133, CD59, glypican-1, CD9, CD63, CD81, integrins, selectins, lectins, cadherins and/or other similar polypeptides known to those of skill in the art. In some aspects, the one or more polypeptides can be a scaffold protein, such as PTGFRN, BSG, IGSF3, IGSF2, ITGB1, ITGA4, SLC3A2, ATP transporter or a fragment thereof. In some aspects, the payload (e.g., IL-12) is fused to Scaffold X, e.g. PTGFRN.

In some aspects, the EV protein is Scaffold Y. In some aspects, the EV protein is polypeptide is BASP1. In some aspects, the one or more polypeptides is a fusion protein comprising the scaffold protein fused to a different protein. In some aspects, the surface protein can be expressed from an exogenous polynucleotide introduced to the producer cells. In some aspects, the surface polypeptide can confer different functionalities to the EV, for example, specific targeting capabilities, delivery functions, enzymatic functions, increased or decreased half-life in vivo, and other desired functionalities known to those of skill in the art.

As previously described, producer cells can be genetically modified to comprise one or more exogenous sequences to produce EVs described herein. The genetically-modified producer cell can contain the exogenous sequence by transient or stable transfection and/or transformation. The exogenous sequence can be transformed as a plasmid. The exogenous sequences can be stably integrated into a genomic sequence of the producer cell, at a targeted site or in a random site. In some aspects, a stable cell line is generated for production of lumen-engineered EVs.

The exogenous sequences can be inserted into a genomic sequence of the producer cell, located within, upstream (5′-end) or downstream (3′-end) of an endogenous sequence encoding an EV protein. Various methods known in the art can be used for the introduction of the exogenous sequences into the producer cell. For example, cells modified using various gene editing methods (e.g., methods using a homologous recombination, transposon-mediated system, loxP-Cre system, CRISPR/Cas9 or TALEN) are within the scope of the present disclosure.

The exogenous sequences can comprise a sequence encoding a scaffold moiety disclosed herein or a fragment or variant thereof. Extra copies of the sequence encoding a scaffold moiety can be introduced to produce an engineered EV described herein (e.g., having a higher density of a scaffold moiety on the exterior surface or on the luminal surface of the EV). An exogenous sequence encoding a modification or a fragment of a scaffold moiety can be introduced to produce a lumen-engineered and/or surface-engineered EV containing the modification or the fragment of the scaffold moiety.

In some aspects, a producer cell disclosed herein is further modified to comprise an additional exogenous sequence. For example, an additional exogenous sequence can be introduced to modulate endogenous gene expression, or produce an EV including a certain polypeptide. In some aspects, the producer cell is modified to comprise two exogenous sequences, one encoding a scaffold moiety (e.g., Scaffold X and/or Scaffold Y), or a variant or a fragment thereof, and the other encoding a molecule linked to the scaffold moiety. In certain aspects, the producer cell can be further modified to comprise an additional exogenous sequence conferring additional functionalities to the EVs. In some aspects, the producer cell is modified to comprise two exogenous sequences, one encoding a scaffold moiety disclosed herein, or a variant or a fragment thereof, and the other encoding a protein conferring the additional functionalities to the EVs. In some aspects, the producer cell is further modified to comprise one, two, three, four, five, six, seven, eight, nine, or ten or more additional exogenous sequences.

In some aspects, EVs of the present disclosure (e.g., surface-engineered and/or lumen-engineered EVs) can be produced from a cell transformed with a sequence encoding a full-length, mature scaffold moiety disclosed herein. Any of the scaffold moieties described herein can be expressed from a plasmid, an exogenous sequence inserted into the genome or other exogenous nucleic acid, such as a synthetic messenger RNA (mRNA).

In certain aspects, the one or more moieties are introduced into the EVs by transfection. In some aspects, the one or more moieties can be introduced into the EVs using synthetic macromolecules such as cationic lipids and polymers (Papapetrou et al., Gene Therapy 12: S118-S130 (2005)). In certain aspects, chemicals such as calcium phosphate, cyclodextrin, or polybrene, can be used to introduce the one or more moieties to the EVs.

In other aspects, one or more scaffold moieties are expressed in the membrane of the EVs by recombinantly expressing the scaffold moieties in the producer cells. The EVs obtained from the producer cells can be further modified to be conjugated to a chemical compound, a nucleic acid, a peptide, a protein, or a linker. In other aspects, the scaffold moiety, e.g., Scaffold X and/or Scaffold Y, is deglycosylated. In some aspects, the scaffold moiety, Scaffold X and/or Scaffold Y, is highly glycosylated, e.g., higher than naturally-occurring Scaffold X and/or Scaffold Y under the same condition.

In certain aspects, one or more moieties can be introduced into the EVs directly after exosome production e.g., loaded into the EVs: for example, passive diffusion, electroporation, chemical or polymeric transfection, viral transduction, mechanical membrane disruption or mechanical shear, or any combination thereof. In some aspects, the one or more moieties and the EV, e.g., exosome, of the present disclosure can be incubated in an appropriate buffer during loading or encapsulation. The term “encapsulated”, or grammatically different forms of the term (e.g., encapsulation, or encapsulating), refers to a status or process of having a first moiety (e.g., STING agonist) inside a second moiety (e.g., an EV, e.g., exosome) without chemically or physically linking the two moieties. In some aspects, the term “encapsulated” can be used interchangeably with “in the lumen of” or “loaded”. Non-limiting examples of encapsulating a first moiety (e.g., STING agonist) into a second moiety (e.g., EVs, e.g., exosomes) are disclosed elsewhere herein. In some aspects, the moiety that can be encapsulated or loaded in the EVs includes a STING agonist. STING agonists refer to an agent that activates a STING pathway. Activation of the STING pathway in DCs results in Type I IFN and pro inflammatory cytokine production via TBK1, IRF3, and NF-κB signaling. Binding of IFN to their receptors on cells results in activation of IFN-stimulated response elements and the transcription of IFN-sensitive genes that result in the immune and inflammatory response. IFN signaling also cross-primes DCs to promote antigen persistence, alters the antigen repertoire available for MHCI presentation, enhances MHCI presentation of antigens, and increases the overall surface expression of MHCI, MHCII, and co-stimulatory molecules CD40, CD80, and CD86. These actions result in increased priming of tumor specific CD8+ T cells and initiation of the adaptive immune response.

In some aspects, a STING agonist useful for the EVs of the present disclosure comprises a cyclic dinucleotide (CDN) and/or a non-cyclic nucleotide. STING agonists used in this disclosure can be cyclic purine dinucleotides such as, but not limited to, cGMP, cyclic di-GMP (c-di-GMP), cAMP, cyclic di-AMP (c-di-AMP), cyclic-GMP-AMP (cGAMP), cyclic di-IMP (c-di-IMP), cyclic AMP-IMP (cAIMP), and any analogue thereof, which are known to stimulate or enhance an immune or inflammation response in a patient. The CDNs may have 2′2′, 2′3′, 2′5′, 3′3′, or 3′5′ bonds linking the cyclic dinucleotides, or any combination thereof. Further non-limiting examples of STING agonists that can be used with the present disclosure include: DMXAA, STING agonist-1, ML RR-S2 CDA, ML RR-S2c-di-GMP, ML-RR-S2 cGAMP, 2′3′-c-di-AM(PS)2, 2′3′-cGAMP, 2′3′-cGAMPdFHS, 3′3′-cGAMP, 3′3′-cGAMPdFSH, cAIMP, cAIM(PS)2, 3′3′-cAIMP, 3′3′-cAIMPdFSH, 2′2′-cGAMP, 2′3′-cGAM(PS)2, 3′3′-cGAMP, and combinations thereof. Non-limiting examples of the STING agonists can also be found at U.S. Pat. No. 9,695,212, WO 2014/189805 A1, WO 2014/179335 A1, WO 2018/100558 A1, U.S. Pat. No. 10,011,630 B2, WO 2017/027646 A1, WO 2017/161349 A1, and WO 2016/096174 A1, each of which is incorporated by reference in its entirety.

Cyclic purine dinucleotides can be modified via standard organic chemistry techniques to produce analogues of purine dinucleotides. Suitable purine dinucleotides include, but are not limited to, adenine, guanine, inosine, hypoxanthine, xanthine, isoguanine, or any other appropriate purine dinucleotide known in the art. The cyclic dinucleotides may be modified analogues. Any suitable modification known in the art may be used, including, but not limited to, phosphorothioate, biphosphorothioate, fluorinate, and difluorinate modifications.

Non cyclic dinucleotide agonists may also be used, such as 5,6-Dimethylxanthenone-4-acetic acid (DMXAA), or any other non-cyclic dinucleotide agonist known in the art.

It is contemplated that any STING agonist can be used. Among the STING agonists are DMXAA, STING agonist-1, ML RR-S2 CDA, ML RR-S2c-di-GMP, ML-RR-S2 cGAMP, 2′3′-c-di-AM(PS)2, 2′3′-cGAMP, 2′3′-cGAMPdFHS, 3′3′-cGAMP, 3′3′-cGAMPdFSH, cAIMP, cAIM(PS)2, 3′3′-cAIMP, 3′3′-cAIMPdFSH, 2′2′-cGAMP, 2′3′-cGAM(PS)2, 3′3′-cGAMP, c-di-AMP, 2′3′-c-di-AMP, 2′3′-c-di-AM(PS)2, c-di-GMP, 2′3′-c-di-GMP, c-di-IMP, c-di-UMP or any combination thereof. In some aspects, the STING agonist is 3′3′-cAIMPdFSH, alternatively named 3-3 cAIMPdFSH. Additional STING agonists known in the art can also be used.

In some aspects, one or more moieties can be introduced into the EVs via an anchoring moiety, e.g., a lipid anchor, e.g., loaded into the EVs: In other aspects, the lipid anchor can be any lipid anchor known in the art, e.g., palmitic acid or glycosylphosphatidylinositols. Under unusual circumstances, e.g., by using a culture medium where myristic acid is limiting, some other fatty acids including shorter-chain and unsaturated, can be attached to the N-terminal glycine. For example, in BK channels, myristate has been reported to be attached posttranslationally to internal serine/threonine or tyrosine residues via a hydroxyester linkage. Membrane anchors known in the art are presented in the following table:

| Modification | Modifying Group |
|---|---|
| S-Palmitoylation | 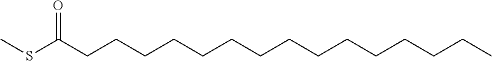 [SMILES: CCCCCCCCCCCCCCCC(=O)SC] |
| N-Palmitoylation | 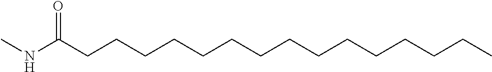 [SMILES: CCCCCCCCCCCCCCCC(=O)NC] |
| N-Myristoylation | 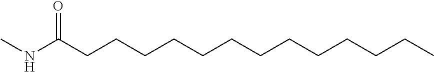 [SMILES: CCCCCCCCCCCCCC(=O)NC] |
| O-Acylation | 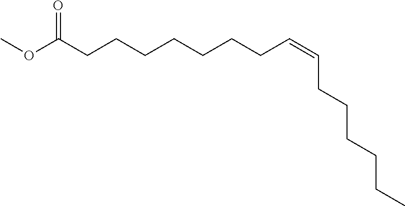 [SMILES: CCCCCC/C=C\CCCCCCCC(=O)OC] |
| Farnesylation | 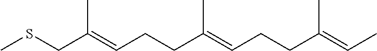 [SMILES: C/C=C(\C)CC/C=C(\C)CC/C=C(\C)CSC] |
| Geranylgeranylation | 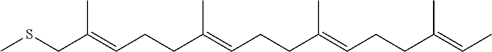 [SMILES: C/C=C(\C)CC/C=C(\C)CC/C=C(\C)CC/C=C(\C)CSC] |
| Cholesterol | 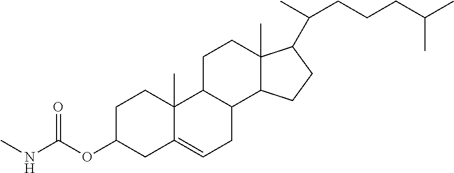 [SMILES: CNC(=O)OC1CCC2(C)C(=CCC3C2CCC2(C)C(C(C)CCCC(C)C)CCC32)C1] |

### III.A. Linkers

In some aspects, a payload, e.g., one or more moieties, can be linked to a scaffold moiety or an anchoring moiety either chemically or non-chemically. In some aspects, a biologically active molecule is linked to a scaffold moiety or an anchoring moiety or an EV via a chemical linker, e.g., a maleimide moiety, a sulfhydryl linker, etc.

In some aspects, a payload is linked to a scaffold moiety or an anchoring moiety on the exterior surface of the EV. In some aspects, the payload is linked to the scaffold moiety or an anchoring moiety on the luminal surface of the EV. In some aspects, the scaffold moiety or an anchoring moiety comprises sterol, GM1, a lipid, a vitamin, a small molecule, a peptide, or a combination thereof. In some aspects, the scaffold moiety or an anchoring moiety comprises cholesterol. In some aspects, the scaffold moiety or an anchoring moiety comprises a phospholipid, a lysophospholipid, a fatty acid, a vitamin (e.g., vitamin D and/or vitamin E), or any combination thereof. In some aspects, the payload is linked to the scaffold moiety or an anchoring moiety by a linker.

In some aspects, a linker can comprise a cholesterol moiety. See, e.g., US 2008/0085869 A1, which is herein incorporated by reference in its entirety.

In some aspects, one or more linkers comprise smaller units (e.g., HEG, TEG, glycerol, C2 to C12 alkyl, and the like) linked together. In some aspects, the linkage is an ester linkage (e.g., phosphodiester or phosphorothioate ester) or other linkage. Examples of non-cleavable linkers that can be used with the present disclosure are known in the art, see, e.g., U.S. Pat. No. 7,569,657 B2; U.S. Pat. No. 8,465,730 B1; U.S. Pat. No. 7,087,229 B2; and U.S. Publ. No. 2014/0193849 A1, each of which is herein incorporated by reference in its entirety. In some aspects, the linker can be, e.g., maleimido caproyl (MC), maleimido propanoyl (MP), methoxyl polyethyleneglycol (MPEG), succinimidyl 4-(N-maleimidomethyl)-cyclohexane-1-carboxylate (SMCC), m-maleimidobenzoyl-N-hydroxysuccinimide ester (MBS), succinimidyl 4-(p-maleimidophenyl)butyrate (SMPB), N-succinimidyl(4-iodoacetyl)aminobenzonate (SIAB), succinimidyl 6-[3-(2-pyridyldithio)-propionamide]hexanoate (LC-SPDP), 4-succinimidyloxycarbonyl-alpha-methyl-alpha-(2-pyridyldithio)toluene (SMPT), etc. (see, e.g., U.S. Pat. No. 7,375,078, which is herein incorporated by reference in its entirety).

In some aspects, the linker comprises acrylic phosphoramidite (e.g., ACRYDITE™), adenylation, azide (NHS Ester), digoxigenin (NHS Ester), cholesterol-TEG, I-LINKER™, an amino modifier (e.g., amino modifier C6, amino modifier C12, amino modifier C6 dT, or Uni-Link™ amino modifier), alkyne, 5′ Hexynyl, 5-Octadiynyl dU, biotinylation (e.g., biotin, biotin (Azide), biotin dT, biotin-TEG, dual biotin, PC biotin, or desthiobiotin), thiol modification (thiol modifier C3 S—S, dithiol or thiol modifier C6 S—S), or any combination thereof. In some aspects, the linker is a cleavable linker. In some aspects, the linker comprises valine-alanine-p-aminobenzylcarbamate or valine-citrulline-p-aminobenzylcarbamate. In some aspects, the linker comprises (i) a maleimide moiety and (ii) valine-alanine-p-aminobenzylcarbamate or valine-citrulline-p-aminobenzylcarbamate.

### IV. Extracellular Vesicles Purified by Present Methods

The present disclosure also includes extracellular vesicles (EVs), e.g., exosomes, purified by the present disclosure. In some aspects, the EVs purified by the present methods include lower impurities, e.g., total nucleic acid impurities, than EVs purified by a process that does not comprise contacting a chromatography resin with a wash buffer disclosed herein, e.g., a wash buffer comprising MgCl₂, CaCl₂, KCl, and/or NaCl.

In some aspects, the present disclosure provides a pharmaceutical composition comprising the purified EVs described herein and a pharmaceutically acceptable carrier. In some aspects, the present disclosure provides a composition comprising EVs and nucleic acid molecule impurities, wherein the nucleic acid molecule impurities are lower than a reference EV composition purified by a process that does not comprise contacting a chromatography resin with a wash buffer disclosed herein, e.g., a wash buffer comprising MgCl₂, CaCl₂, KCl, and/or NaCl. In some aspects, the present disclosure provides a composition comprising EVs and nucleic acid molecule impurities, wherein the nucleic acid molecule impurities are at least about 5%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, or at least about 40% lower in the purified EV composition compared to a reference EV composition purified by a process that does not comprise contacting a chromatography resin with a wash buffer disclosed herein, e.g., a wash buffer comprising MgCl₂, CaCl₂, KCl, and/or NaCl.

In some aspects, the nucleic acid molecule impurities are at least about 5%, e.g., 5% to 10%, 5% to 20%, 5% to 25%, or 5% to 30%, lower in the purified EV composition compared to a reference EV composition purified by a process that does not comprise contacting a chromatography resin with a wash buffer disclosed herein, e.g., a wash buffer comprising MgCl₂, CaCl₂, KCl, and/or NaCl. In some aspects, the nucleic acid molecule impurities are at least about 10%, e.g., 10% to 15%, 10% to 20%, 10% to 25%, 10% to 30%, 10% to 35%, 10% to 30%, 10% to 95%, 20% to 90%, 50% to 90%, or 80% to 90% lower in the purified EV composition compared to a reference EV composition purified by a process that does not comprise contacting a chromatography resin with a wash buffer disclosed herein, e.g., a wash buffer comprising MgCl₂, CaCl₂, KCl, and/or NaCl. In some aspects, the protein impurities are at least about 11% lower in the purified EV composition compared to a reference EV composition purified by a process that does not comprise contacting a chromatography resin with a wash buffer disclosed herein, e.g., a wash buffer comprising MgCl₂, CaCl₂, KCl, and/or NaCl. In some aspects, the nucleic acid molecule impurities are at least about 12% lower in the purified EV composition compared to a reference EV composition purified by a process that does not comprise contacting a chromatography resin with a wash buffer disclosed herein, e.g., a wash buffer comprising MgCl₂, CaCl₂, KCl, and/or NaCl. In some aspects, the nucleic acid molecule impurities are at least about 13% lower in the purified EV composition compared to a reference EV composition purified by a process that does not comprise contacting a chromatography resin with a wash buffer disclosed herein, e.g., a wash buffer comprising MgCl₂, CaCl₂, KCl, and/or NaCl. In some aspects, the nucleic acid molecule impurities are at least about 14% lower in the purified EV composition compared to a reference EV composition purified by a process that does not comprise contacting a chromatography resin with a wash buffer disclosed herein, e.g., a wash buffer comprising MgCl₂, CaCl₂, KCl, and/or NaCl. In some aspects, the nucleic acid molecule impurities are at least about 15%, e.g., 15% to 20%, 15% to 25%, 15% to 30%, 15% to 35%, 15% to 40%, 20% to 25%, 20% to 30%, 20% to 35%, or 20% to 40%, lower in the purified EV composition compared to a reference EV composition purified by a process that does not comprise contacting a chromatography resin with a wash buffer disclosed herein, e.g., a wash buffer comprising MgCl₂, CaCl₂, KCl, and/or NaCl.

In some aspects, compositions comprising the purified EVs have an EV concentration that is approximately the same as the concentration of EVs in a reference composition comprising EVs purified by a process that does not comprise contacting a chromatography resin with a wash buffer disclosed herein, e.g., a wash buffer comprising MgCl₂, CaCl₂, KCl, and/or NaCl. In some aspects, compositions comprising the purified EVs has an EV concentration that is more than about 99% of the concentration of EVs in a reference composition comprising EVs purified by a process that does not comprise contacting a chromatography resin with a wash buffer disclosed herein, e.g., a wash buffer comprising MgCl₂, CaCl₂, KCl, and/or NaCl. In some aspects, compositions comprising the purified EVs has an EV concentration that is more than about 98% of the concentration of EVs in a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, compositions comprising the purified EVs has an EV concentration that is more than about 97% of the concentration of EVs in a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, compositions comprising the purified EVs has an EV concentration that is more than about 96% of the concentration of EVs in a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, compositions comprising the purified EVs has an EV concentration that is more than about 95% of the concentration of EVs in a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, compositions comprising the purified EVs has an EV concentration that is more than about 90% of the concentration of EVs in a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, compositions comprising the purified EVs has an EV concentration that is more than about 85% of the concentration of EVs in a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, compositions comprising the purified EVs has an EV concentration that is more than about 80% of the concentration of EVs in a reference composition comprising EVs purified by a process that does not comprise contacting a chromatograph resin with a wash buffer disclosed herein, e.g., a wash buffer comprising MgCl₂, CaCl₂, KCl, and/or NaCl.

In some aspects, compositions comprising the purified EVs have a higher potency than a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, the potency of the composition comprising the purified EVs is at least about 5%, e.g., 5% to 10%, 5% to 15%, 5% to 20%, 5% to 25%, 5% to 30%, 5% to 35%, 5% to 40%, 5% to 45%, 5% to 50%, e.g., 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% higher than that of a reference composition comprising EVs purified by a process that does not comprise contacting a chromatograph resin with a wash buffer disclosed herein, e.g., a wash buffer comprising MgCl₂, CaCl₂, and/or NaCl. In some aspects, the potency of the composition comprising the purified EVs is at least about 10%, e.g., 10% to 15%, 10% to 20%, 10% to 25%, 10% to 30%, 10% to 35%, 10% to 40%, 10% to 45%, 10% to 50%, 10% to 55%, or 10% to 60%, e.g., 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, or 60%, higher than that of a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, the potency of the composition comprising the purified EVs is at least about 11% higher than that of a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, the potency of the composition comprising the purified EVs is at least about 15%, e.g., 15% to 20%, 15% to 25%, 15% to 30%, 15% to 35%, 15% to 40%, 15% to 45%, 15% to 50%, 15% to 55%, or 15% to 60%, e.g., 16%, 17%, 18%, 19%, 20%, 21%, 22%, 23%, 24%, 25%, 26%, 27%, 28%, 29%, 30%, 35%, 40%, 45%, 50%, 55%, or 60%, higher than that of a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, the potency of the composition comprising the purified EVs is at least about 20%, e.g., 20% to 25%, 20% to 30%, 20% to 35%, 20% to 40%, 20% to 45%, 20% to 50%, 20% to 55%, or 20% to 60%, e.g., 20%, 21%, 22%, 23%, 24%, 25%, 26%, 27%, 28%, 29%, 30%, 35%, 40%, 45%, 50%, 55%, or 60%, higher than that of a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, the potency of the composition comprising the purified EVs is at least about 25%, e.g., 25% to 30%, 25% to 35%, 25% to 40%, 25% to 45%, 25% to 50%, 25% to 55%, or 25% to 60%, e.g., 25%, 26%, 27%, 28%, 29%, 30%, 35%, 40%, 45%, 50%, 55%, 60%, 65%, 70%, 75%, or 80%, higher than that of a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, the potency of the composition comprising the purified EVs is at least about 30%, e.g., 30% to 35%, 30% to 40%, 30% to 45%, 30% to 50%, 30% to 55%, or 30% to 60%, e.g., 30%, 35%, 40%, 45%, 50%, 55%, 60%, 65%, 70%, 75%, 80%, 80%, 85%, or 90% higher than that of a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, the potency of the composition comprising the purified EVs is at least about 35%, e.g., 35% to 40%, 35% to 45%, 35% to 50%, 35% to 55%, or 35% to 60%, e.g., 35%, 40%, 45%, 50%, 55%, 60%, 65%, 70%, 75%, 80%, 80%, 85%, or 90% higher than that of a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, the potency of the composition comprising the purified EVs is at least about 40%, e.g., 40% to 45%, 40% to 50%, 40% to 55%, or 40% to 60%, e.g., 40%, 45%, 50%, 55%, 60%, 65%, 70%, 75%, 80%, 80%, 85%, or 90%, higher than that of a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, the potency of the composition comprising the purified EVs is at least about 45%, e.g., 45% to 50%, 45% to 55%, or 45% to 60%, e.g., 45%, 50%, 55%, 60%, 65%, 70%, 75%, 80%, 80%, 85%, or 90% higher than that of a reference composition comprising EVs purified by a process that does not comprise contacting a chromatograph resin with a wash buffer disclosed herein, e.g., a wash buffer comprising MgCl₂, CaCl₂, KCl, and/or NaCl. In some aspects, the potency of the composition comprising the purified EVs is at least about 50% higher than that of a reference composition comprising EVs purified by a process that does not comprise contacting a chromatograph resin with a wash buffer disclosed herein, e.g., a wash buffer comprising MgCl₂, CaCl₂, KCl, and/or NaCl.

In some aspects, the purified EVs according to the present disclosure is at least 75% pure. In some aspects, the purified EVs according to the present disclosure is at least about 80% pure. In some aspects, the purified EVs according to the present disclosure is at least about 85% pure. In some aspects, the purified EVs according to the present disclosure is at least about 90% pure. In some aspects, the purified EVs according to the present disclosure is at least about 95% pure. In some aspects, the purified EVs according to the present disclosure is at least about 96% pure. In some aspects, the purified EVs according to the present disclosure is at least about 97% pure. In some aspects, the purified EVs according to the present disclosure is at least about 98% pure. In some aspects, the purified EVs according to the present disclosure is at least about 99% pure. In some aspects, the purified EVs according to the present disclosure is about 100% pure.

In some aspects, a composition comprising the purified EVs of the present disclosure further comprises a saccharide. In some aspects, a composition comprising the purified EVs of the present disclosure further comprises sodium chloride. In some aspects, a composition comprising the purified EVs of the present disclosure further comprises a potassium phosphate. In some aspects, a composition comprising the purified EVs of the present disclosure further comprises a sodium phosphate. In some aspects, a composition comprising the purified EVs of the present disclosure further comprises one or more of a saccharide, sodium chloride, a potassium phosphate, and a sodium phosphate. In some aspects, a composition comprising the purified EVs of the present disclosure further comprises a saccharide, sodium chloride, a potassium phosphate, and a sodium phosphate.

In some aspects, the present disclosure provides a method of administering a composition comprising purified EVs to a subject in need thereof. In some aspects, the present disclosure provides a method of treating a disease or condition in a subject in need thereof comprising administering to the subject a composition comprising purified EVs.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

### EXAMPLES

The following examples are put forth so as to provide those of ordinary skill in the art with a complete disclosure and description of how to make and use the aspects described herein, and are not intended to limit the scope of the appended claims, nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations can be used, e.g., s or sec, second(s); min, minute(s); h or hr, hour(s).

The aspects described herein employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); AL. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 21th Edition (Easton, Pennsylvania: Mack Publishing Company, 2005); Carey and Sundberg Advanced Organic Chemistry 3rd Ed. (Plenum Press) Vols A and B(1992).

### Example 1: Wash Buffer Assessment of DNA Clearance Processes

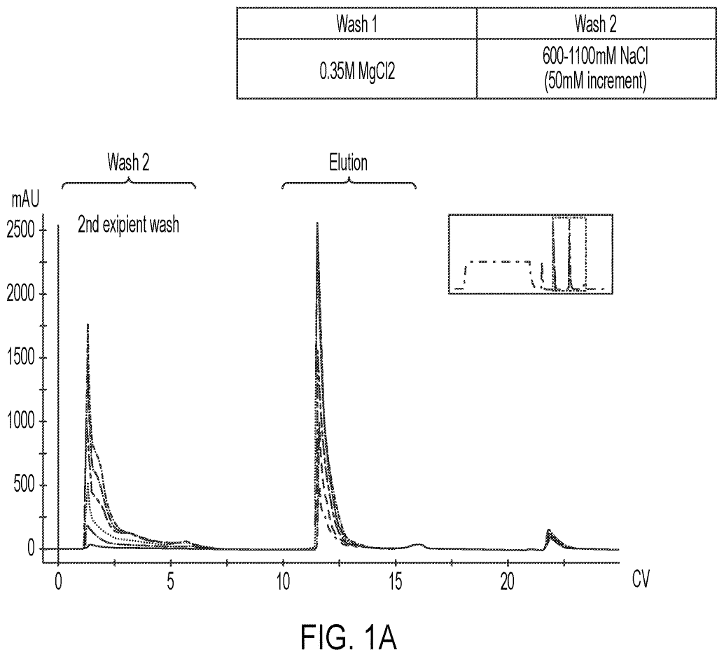

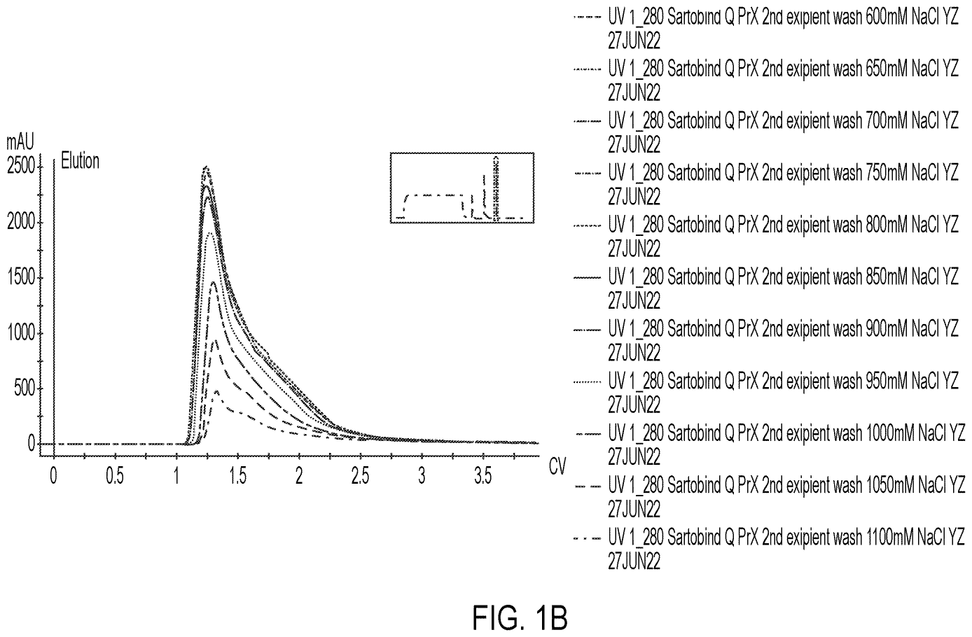

In the present example, the performances of an experimental DNA clearance process was evaluated using exosome samples. Exosome samples were prepared, and the sample was filtered (NaCl, 60LA, XLG 0.2 um) (FIG. 1). Subsequently, individual AEX runs were performed on each individual exosome sample, where each of the different individual AEX runs for each individual exosome sample was performed using a different wash buffer.

To test the effect of varying the concentration of NaCl in a second wash, exosome samples were applied to an AEX matrix and washed first with a buffer comprising 350 mM MgCl₂ (“Wash 1”) and then with a buffer comprising between 600 mM to 1100 mM NaCl (“Wash 2”), in 50 mM increments. Increasing the concentration of NaCl in Wash 2 resulted in more material desorbing during the second wash (FIG. 1A) and less material desorbing during elution (FIGS. 1A and 1). The loss of exosomes in the elution was most pronounced at NaCl concentrations in Wash 2 of 850 mM or greater.

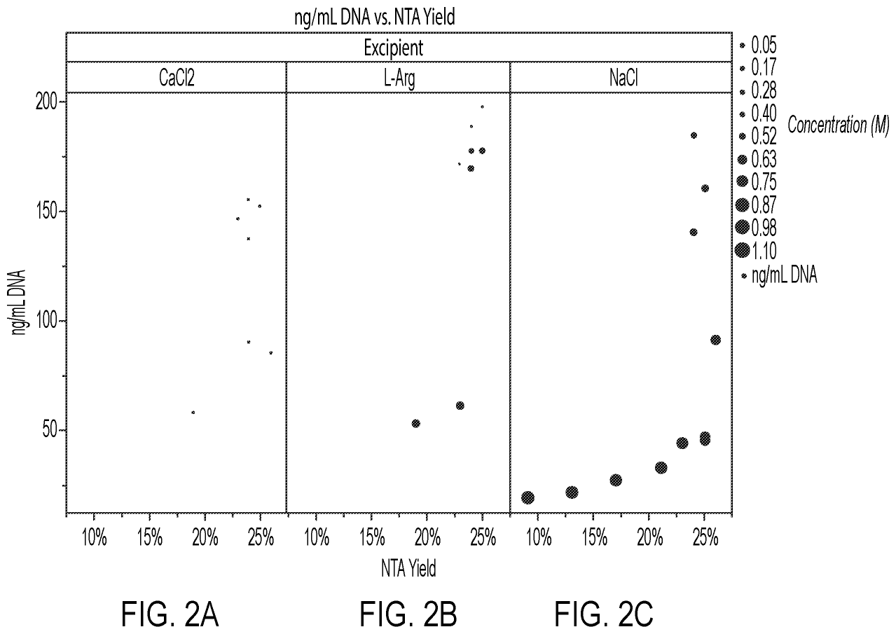

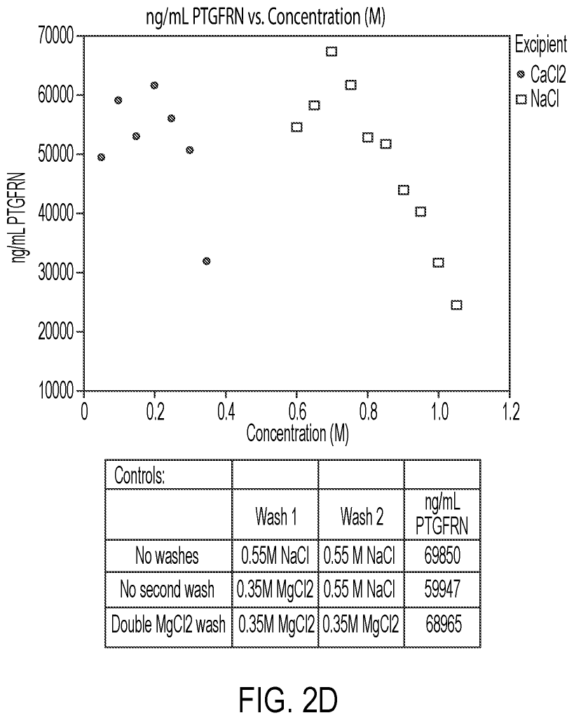

Next, different salts and concentrations for Wash 2 were evaluated. Exosome samples were applied to an AEX matrix and washed with 5 MV of 350 MgCl₂, followed by 5 MV of a second wash comprising CaCl₂ (FIG. 2A), L-Arginine (FIG. 2B), or NaCl (FIG. 2C), at increasing concentrations. NTA yield was used as a measurement of exosome concentration in the eluted product (FIGS. 2A-2C). NaCl provided the best DNA removal with minimal product yield loss (FIG. 2C). Particle count was reduced at high salt concentrations due to increased loss during elution. Controls included (i) first and second washes with 550 mM NaCl (16,294 ng/mL residual DNA; 26% NTA yield), (ii) a 350 mM MgCl₂ Wash 1 followed by a 550 mM NaCl Wash 2 (163 ng/mL residual DNA; 25% NTA yield), and (iii) first and second washes with 350 mM MgCl₂ (74 ng/mL residual DNA; 24% NTA yield).

Removal of PTGFRN, a component of exosomes confirms that at excessive excipient concentrations, exosomes are desorbed from the anion exchange chromatography resin in Wash 2 (FIG. 2D). Matrices washed with (i) first and second washes comprising 550 mM NaCl were observed to have 69,850 ng/mL PTGFRN, (ii) a 350 mM MgCl₂ Wash 1 followed by a 550 mM NaCl Wash 2 were observed to have 59,947 ng/mL PTGFRN, or (iii) first and second washes comprising 350 mM MgCl₂ 68,965 ng/mL PTGFRN.

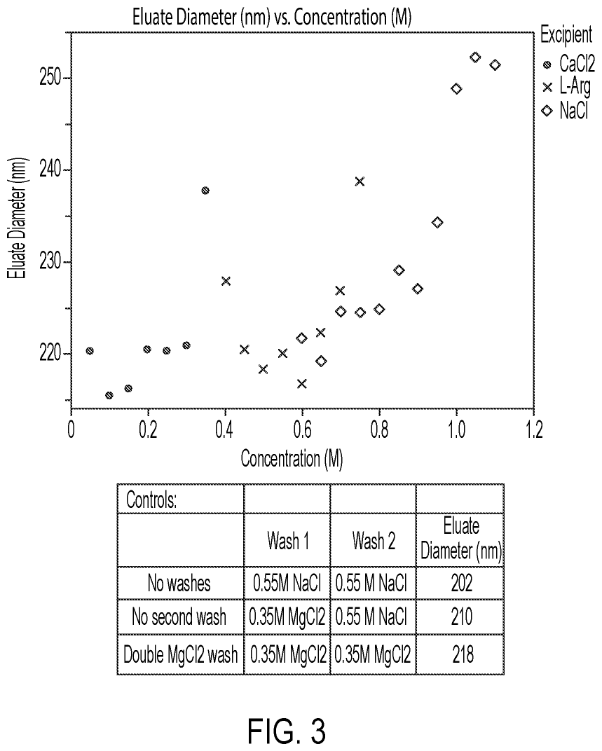

Next, the effect of the various washes on eluate particle size was evaluated at fixed elution conditions. Exosome samples were applied to an AEX matrix and washed with 350 mM MgCl₂ (Wash 1) followed by a second wash comprising CaCl₂ (circles), L-arginine (x's), or NaCl (diamonds) at varying concentrations (FIG. 3) Higher concentrations of excipient washes removed more smaller size particles during wash (FIG. 3). Particle size (exosomes) shows subsequent increase during high salt elution (FIG. 3).

Wash volume (5 or 10 MV) or wash sequence didn't change DNA clearance (Table 1). Adding a second excipient wash step increased DNA removal by two-fold, and the use of 350 mM MgCl₂ or 250 mM CaCl₂ resulted in similar DNA removal. Finally, using 800 mM NaCl in either the first or second wash achieved four-fold great DNA removal over the control.

TABLE 1

| | | Wash 1 | | | Wash 2 | DNA |
|---|---|---|---|---|---|---|
| Entry | Wash 1 | Vol (MV) | | Wash 2 | Vol (MV) | (ng/mL) |
| 1 | 350 mM MgCl2 | 5 | Re-equil | 550 mM NaCl | 10 | 125 |
| 2 | 350 mM MgCl2 | 10 | (550 mM | 550 mM NaCl | 10 | 124 |
| 3 | 350 mM MgCl2 | 10 | NaCl) | 350 mM MgCl2 | 5 | 62 |
| 4 | 350 mM MgCl2 | 10 | | 350 mM MgCl2 | 10 | 63 |
| 5 | 350 mM MgCl2 | 10 | | 250 mM CaCl2 | 5 | 62 |
| 6 | 350 mM MgCl2 | 10 | | 250 mM CaCl2 | 10 | 72 |
| 7 | 250 mM CaCl2 | 10 | | 350 mM MgCl2 | 10 | 90 |
| 8 | 350 mM MgCl2 | 10 | | 800 mM NaCl | 5 | 36 |
| 9 | 350 mM MgCl2 | 10 | | 800 mM NaCl | 10 | 24 |
| 10 | 800 mM NaCl | 10 | | 350 mM MgCl2 | 10 | 27 |

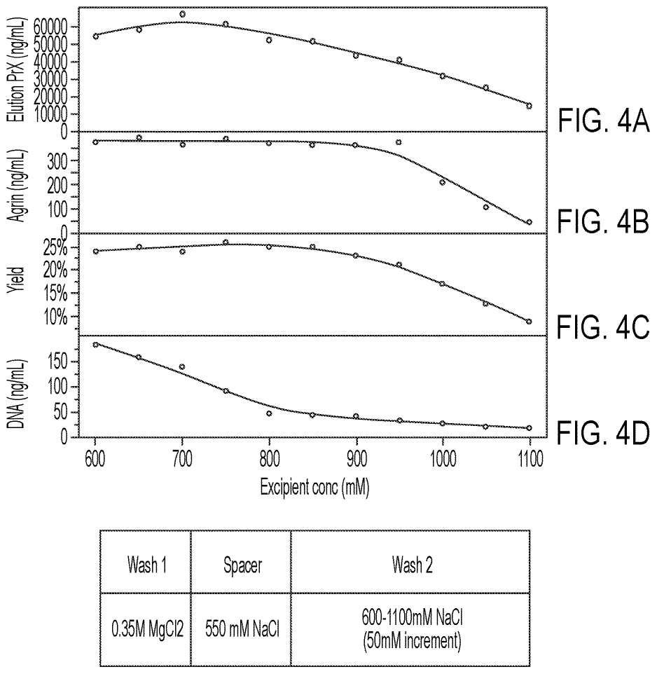

The concentrations of DNA, agrin (another impurity), Protein X (as an indicator of exosomes), and NTA particle recovery (% yield) were evaluated for matrices washed with a Wash 1 comprising 350 mM MgCl₂ and a Wash 2 comprising an increasing concentration of NaCl. DNA was selectively removed with increasing salt in Wash 2 whereas PrX (exosomes), agrin (impurity), and particles remained bound (FIGS. 4A-4D).

Next, the sequence of washes and utility of spacer washes (Eq; 550 mM NaCl) were evaluated. Study shows that low levels of DNA could be achieved without necessitating spacer washes. Fewer washes reduces buffer consumption and speeds up the process. Eq after two washes and before elution was found to not be necessary. 350 mM MgCl₂ (Wash 1) followed by 800 mM NaCl (Wash 2) performed comparable to the best sequence, and minimized the number of wash steps.

TABLE 2

| | | | | DNA in elution |
|---|---|---|---|---|
| Entry | Wash conditions | | | (ng/mL) |
| 1 | Load | Eq-Mg-Eq | Elution | 400 |
| 2 | | Eq-Mg-Eq-NaCl-Eq | | 180 |
| 3 | | Mg—NaCl (selected) | | 169 |
| 4 | | Eq-Mg—NaCl-Eq | | N/A |
| 5 | | Mg-Eq-NaCl | | 147 |
| 6 | | Mg—NaCl-Eq | | 193 |
| 7 | | NaCl—Mg-Eq | | 197 |

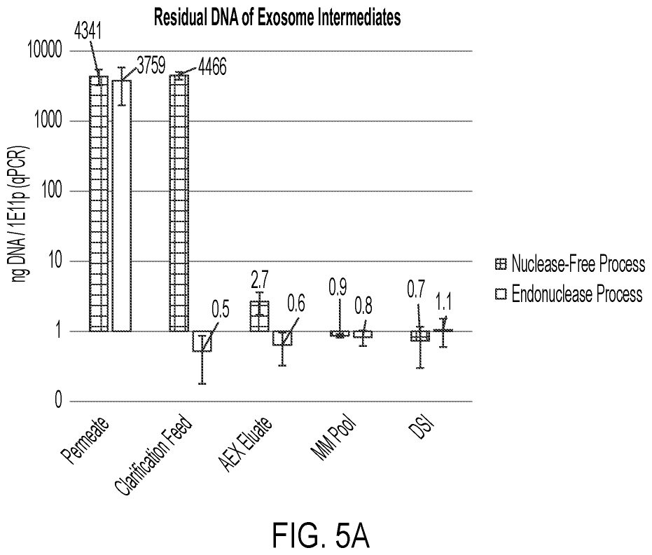

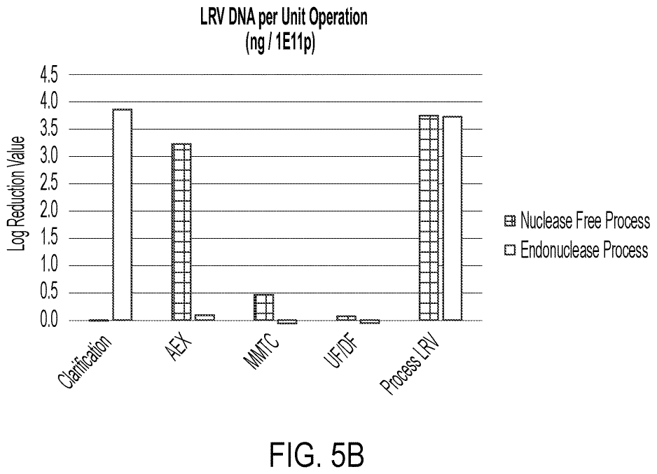

MMTC gave the same DNA clearance even though the load differed by two- to three-fold in DNA levels. The amount of DNA in the AEX pool was altered by changing wash steps. The data in Table 3 show that the downstream MMTC process is able to compensate for variability in DNA clearance across AEX, which is evidence of robustness across entire purification process. Similar results are shown in FIGS. 5A and 5B, which show that the level of residual DNA is reduced using the nuclease-free methods disclosed herein to levels similar to those observed following a nuclease treatment.

TABLE 3

| | MMTC load DNA | MMTC elution DNA |
|---|---|---|
| AEX condition | (ng/mL) | (ng/mL) |
| Mg-0.55M NaCl- | 78 | 3 |
| Elution | | |
| Mg-0.55M NaCl- | 31 | 4 |
| 0.8M NaCl-0.55M | | |
| NaCl-Elution | | |

What is claimed is:

1. A method of preparing purified extracellular vesicles (EVs) from a sample comprising EVs and one or more nucleic acid molecules, comprising: contacting a chromatography resin associated with the sample with a wash buffer; wherein the wash buffer comprises MgCl₂, CaCl₂, KCl, and/or NaCl and does not comprise a nuclease.

2. A method of reducing the concentration of residual nucleic acid molecule in a sample comprising extracellular vesicles (EVs), comprising contacting a chromatography resin associated with the sample with a wash buffer; wherein the wash buffer comprises MgCl₂, CaCl₂, KCl and/or NaCl and does not comprise a nuclease.

3. The method of claim 1 or 2, wherein the sample does not come into a contact with a nuclease during the method.

4. The method of any one of claims 1 to 3, wherein the chromatography resin is selected from the group consisting of a cation exchange (CEX) resin, an anion exchange (AEX) resin, an affinity chromatography resin, a pseudo affinity chromatography resin, a hydrophobic interaction resin, a hydrophobic charge induction chromatography resin, a mixed mode resin, an immobilized metal affinity resin, a ceramic hydroxyapatite resin, a fluoro hydroxyapatite resin, a ceramic fluoroapatite and any combination thereof.

5. The method of any one of claims 1 to 4, wherein the chromatography resin comprises an AEX resin.

6. The method of any one of claims 1 to 4, wherein the chromatography resin comprises a CEX resin.

7. The method of any one of claims 1 to 4 wherein the chromatography resin comprises an affinity chromatography resin.

8. The method of any one of claims 1 to 6, wherein the nuclease is an endonuclease or exo nuclease.

9. The method of any one of claims 1 to 8, wherein the nuclease is selected from salt active nuclease (SAN), benzonase, denarase, kryptonase, and any combination thereof.

10. The method of any one of claims 1 to 9, wherein the wash buffer comprises MgCl₂, and wherein the concentration of Mg²⁺ in the wash buffer is at least about 200 mM to about 500 mM.

11. The method of any one of claims 1 to 10, wherein the wash buffer comprises MgCl₂, and wherein the concentration of Mg²⁺ in the wash buffer is at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, or at least about 500 mM Mg₂⁺.

12. The method of claim 10 or 11, wherein the concentration of the Mg²⁺ in the wash buffer is at least about 350 mM Mg₂⁺.

13. The method of any one of claims 1 to 11, wherein the wash buffer comprises at least about 350 mM MgCl₂.

14. The method of any one of claims 1 to 9, wherein the wash buffer comprises CaCl₂, and wherein the concentration of Ca²⁺ in the wash buffer is at least about 100 mM to about 400 mM.

15. The method of any one of claims 1 to 9 and 14, wherein the wash buffer comprises CaCl₂, and wherein the concentration of Ca²⁺ in the wash buffer is at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, or at least about 400 mM Ca²⁺.

16. The method of claim 14 or 15, wherein the concentration of the Ca²⁺ in the wash buffer is at least about 250 mM Ca²⁺.

17. The method of any one of claims 1 to 9 and 14, wherein the wash buffer comprises at least about 250 mM CaCl₂.

18. The method of any one of claims 1 to 9, wherein the wash buffer comprises at least about 600 mM to about 1 M NaCl.

19. The method of any one of claims 1 to 9 and 18, wherein the wash buffer comprises NaCl at a concentration of at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1 M NaCl.

20. The method of any one of claims 1 to 9, 18, and 19, wherein the wash buffer comprises at least about 800 mM NaCl.

21. The method of any one of claims 1 to 9, wherein the wash buffer comprises at least about 600 mM to about 1 M KCl.

22. The method of any one of claims 1 to 9 and 21, wherein the wash buffer comprises KCl at a concentration of at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1 M KCl.

23. The method of any one of claims 1 to 9, 21, and 22, wherein the wash buffer comprises at least about 800 mM KCl.

24. The method of any one of claims 1 to 24, wherein the wash buffer is contacted with the chromatography resin associated with the sample at least 2 times, at least 3 times, at least 4 times, or at least 5 times.

25. The method of any one of claims 1 to 24, further comprising contacting the chromatography resin associated with the sample with a second wash buffer, wherein the second wash buffer does not comprise a nuclease.

26. The method of claim 25, wherein the second wash buffer is different from the wash buffer.

27. The method of claim 25 or 26, wherein the second wash buffer comprises MgCl₂, CaCl₂, KCl, and/or NaCl.

28. The method of any one of claims 25 to 27, wherein the second wash buffer comprises at least about 600 mM to about 1 M NaCl.

29. The method of any one of claims 25 to 28, wherein the second wash buffer comprises NaCl at a concentration of at least about 600 mM, at least about 625 mM, at least about 650 mM, at least about 675 mM, at least about 700 mM, at least about 725 mM, at least about 750 mM, at least about 775 mM, at least about at least about 800 mM, at least about 825 mM, at least about 850 mM, at least about 875 mM, at least about 900 mM, at least about 925 mM, at least about 950 mM, at least about 975 mM, or at least about 1 M NaCl.

30. The method of any one of claims 22 to 26, wherein the second wash buffer comprises at least about 800 mM NaCl.

31. The method of any one of claims 25 to 30, wherein:
(i) the wash buffer comprises MgCl₂, wherein the concentration of Mg²⁺ in the wash buffer is at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, or at least about 500 mM Mg²⁺; and
(ii) the second wash buffer comprises at least about 600 mM to about 1 M NaCl.

32. The method of claim 31, wherein:
1. the wash buffer comprises MgCl₂, wherein the concentration of Mg²⁺ in the wash buffer is at least about 350 mM Mg²⁺, and
2. the concentration of NaCl in the second wash buffer is about 800 mM NaCl.

33. The method of any one of claims 25 to 27, wherein:
(i) the wash buffer comprises at least about 600 mM to about 1 M NaCl; and
(ii) the second wash buffer comprises MgCl₂, wherein the concentration of Mg²⁺ in the wash buffer is at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, or at least about 500 mM Mg₂⁺.

34. The method of any one of claims 22 to 27, wherein:
(i) the wash buffer comprises at least about 800 mM NaCl; and
(ii) the second wash buffer comprises MgCl₂, wherein the concentration of Mg²⁺ in the wash buffer is at least about 350 mM.

35. The method of any one of claims 25 to 27, wherein the second wash buffer comprises at least about 600 mM to about 1 M KCl.

36. The method of any one of claims 25 to 27 and 35, wherein the second wash buffer comprises KCl at a concentration of at least about 600 mM, at least about 625 mM, at least about 650 mM, at least about 675 mM, at least about 700 mM, at least about 725 mM, at least about 750 mM, at least about 775 mM, at least about at least about 800 mM, at least about 825 mM, at least about 850 mM, at least about 875 mM, at least about 900 mM, at least about 925 mM, at least about 950 mM, at least about 975 mM, or at least about 1 M KCl.

37. The method of any one of claims 25 to 27, 35, and 36, wherein the second wash buffer comprises at least about 800 mM KCl.

38. The method of any one of claims 1 to 37, further comprising eluting the EVs from the chromatography resin by contacting the chromatography resin associated with a sample with an elution buffer, wherein the eluting occurs after contacting the chromatography resin with the wash buffer.

39. The method of claim 38, wherein the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M.

40. The method of claim 38 or 39, wherein the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M NaCl.

41. The method of any one of claims 38 to 40, wherein the elution buffer comprises at least about 1.2 M NaCl.

42. The method of any one of claims 38 to 40, wherein the elution buffer comprises at least about 1.4 M NaCl.

43. The method of claim 38 or 39, wherein the elution buffer comprises a salt concentration of at least about 1.0 M, at least about 1.1 M, at least about 1.2 M, at least about 1.3 M, at least about 1.4 M, at least about 1.5 M, at least about 1.6 M, at least about 1.7 M, at least about 1.8 M, at least about 1.9 M, at least about 2.0 M, at least about 2.5 M, at least about 3.0 M, at least about 3.5 M, at least about 4.0 M, at least about 4.5 M, or at least about 5.0 M KCl.

44. The method of any one of claims 38 to 40, wherein the elution buffer comprises at least about 1.2 M KCl.

45. The method of any one of claims 38 to 40, wherein the elution buffer comprises at least about 1.4 M KCl.

46. The method of any one of claims 38 to 45, wherein the elution buffer releases one or more EVs from the chromatography resin.

47. The method of any one of claims 38 to 46, further comprising collecting an eluent after contacting the chromatography resin with the elution buffer.

48. The method of claim 47, wherein the eluent comprises one or more EVs.

49. The method of claim 47 or 48, wherein the sample associated with the chromatography resin comprises a starting concentration of the one or more nucleic acid molecules, and wherein the eluent comprises an eluted concentration of the one or more nucleic acid molecules, wherein the eluted concentration of the one or more nucleic acid molecules is less than about 10%, less than about 5%, less than about 1%, less than about 0.5%, less than about 0.1%, less than about 0.05%, less than about 0.01%, less than about 0.001%, or less than about 0.0001% that of the starting concentration of the one or more nucleic acid molecules.

50. The method of any one of claims 1 to 49, further comprising subjecting the sample to one or more additional chromatography resins.

51. The method of claim 50, wherein the one or more additional chromatography resins comprises an anion exchange chromatography (AEX) resin, a cation exchange chromatography (CEX) resin, a mixed mode chromatography (MMC) resin, hydrophobic charge induction chromatography resin, a hydrophobic interaction chromatography resin, an immobilized metal affinity chromatography (IMAC), a ceramic hydroxyapatite resin, a fluoro hydroxyapatite resin, a ceramic fluoroapatite resin, an affinity chromatography resin, a pseudo affinity chromatography resin, or any combination thereof.

52. The method of any one of claims 1 to 51, wherein the EV is an exosome.

53. A composition comprising extracellular vesicles prepared by the method of any one of claims 1 to 52.

54. The composition of claim 53, which further comprises:
a. a saccharide,
b. sodium chloride,
c. a potassium phosphate,
d. a sodium phosphate, and
e. any combination thereof.

55. A method of treating a disease or condition in a subject in need thereof comprising administering the composition of claim 53 or 54.
